(19) United States
(12) Patent Application Publication
(10) Pub. No.: US 2024/0317874 A1
(43) Pub. Date: Sep. 26, 2024

(54) ANTI-OX40 MONOCLONAL ANTIBODY AND METHODS OF USE THEREOF

(71) Applicant: HIFIBIO (HK) LIMITED, Central (HK)

(72) Inventors: Francisco Adrian, Cambridge, MA (US); Liang Schweizer, Cambridge, MA (US); Qian Zhang, Cambridge, MA (US); Yun-Yueh Lu, Cambidge (CN); Andreas Raue, Cambridge, MA (US)

(21) Appl. No.: 18/572,710

(22) PCT Filed: Jun. 28, 2022
(86) PCT No.: PCT/IB2022/000372

§ 371 Date: Dec. 20, 2023

Related U.S. Application Data
(60) Provisional application No. 63/216,189, filed on Jun. 29, 2021.

(51) Int. Cl. C07K 16/28; A61K 39/00; A61K 47/68; A61P 35/00

(52) U.S. Cl. CPC C07K 16/2878; A61K 47/6849; A61P 35/00; A61K 2039/505; C07K 2317/732; C07K 2317/75

(57) ABSTRACT

Provided is a novel anti-OX40 antibody, including the compositions of anti-OX40 antibody, a polynucleotide encoding the anti-OX40 antibody, methods of preparing the anti-OX40 antibody, and methods of using the anti-OX40 antibody.

### REFERENCE TO RELATED APPLICATION

The application claims priority to, and the benefit of the filing date of U.S. Provisional Patent Application No. 63/216,189, filed on Jun. 29, 2021, the entire contents of which, including all drawings and sequence listing thereof, in any language, are incorporated herein by reference.

### TECHNICAL FIELD

The present invention relates to a novel anti-OX40 antibody comprising the compositions of anti-OX40 antibody, a polynucleotide encoding the anti-OX40 antibody, methods of preparing the anti-OX40 antibody, and methods of using the anti-OX40 antibody for the therapeutic treatment of diseases.

### REFERENCE TO THE SEQUENCE LISTING

The instant application contains a Sequence Listing which has been submitted electronically and is hereby incorporated by reference in its entirety. A copy of the Sequence Listing created on Jun. 28, 2022 was named 131206-00920_SL, and is 33,739 bytes in size.

### BACKGROUND OF THE INVENTION

The use of biological agents has improved the anti-tumor immune response in patients with solid tumors. For example, two anti-PD-1 monoclonal antibodies: nivolumab (OPDIVO®) and pembrolizumab (KEYTRUDA®) have been approved in the United States and Europe for the treatment of diseases such as unresectable, metastatic melanoma, and metastatic non-small cell lung cancer. Traditionally, the anti-tumor response of these drugs is assessed by the improvement of progression-free survival and/or overall survival of the patients. With higher healthcare standard and expectations, there is an emerging need for better anti-cancer products and strategies.

PD-1 and CTLA-4 play an immunosuppressive effect in the process of T cell activation, thereby inhibiting the immune function of T cells on tumor cells. Monoclonal antibodies could restore the anti-tumor immune response of T cells by blocking these two immunosuppressive targets. In addition to the inhibition of said immunosuppressive checkpoints, activation of stimulatory checkpoints is becoming the targets for the development of new drugs.

Stimulatory immune checkpoint molecules mainly refer to the ligand-receptor pairs that exert stimulatory effects on T cell activation. These molecules include but not limited to OX40, CD40, 4-1BB, and GITR, which belong to the tumor necrosis factor receptor (TNFR) family and play a role in T cell proliferation, activation, and differentiation.

OX40 receptor, also known as CD134 and TNFRSF4 (Tumor Necrosis Factor Receptor Superfamily Member 4), is a member of the TNFR super family receptor. Unlike other constitutive T cell costimulatory receptor, such as CD28, OX40 is not expressed on naïve T cells. OX40 is a secondary costimulatory immune checkpoint molecule that is expressed predominantly on activated T cells at 24 to 72 hours after activation. Similarly, OX40L (CD252 or TNFSF4), a ligand of OX40, is not expressed on resting antigen presenting cells, but expressed after activation. Expression of OX40 depends on full activation of T cells.

OX40 binds to its ligand OX40L and activities the co-stimulatory signals by recruiting TNFR molecules to the intracellular region to form IKKα-IKKβ and PI3k-PKB(Akt) signaling complex. OX40 and TCR have synergic effects in activating nuclear factor-activated T cell (NFAT) signals by increasing intracellular Ca²⁺ level through an unknown mechanism, leading to nuclear entry of NFAT. OX40 also plays a pivotal role in activation, potentiation, proliferation, and survival of T cells through the activation of PI3k/PKB and NFAT pathways, the classic NF-κB1 pathway, or the non-canonical NF-κB2 pathway. Additionally, OX40 down-regulates the expression of CTLA-4 and Foxp3.

The mechanisms of OX40-OX40L complex in enhancing immune responses are two folds. First, it increases the secretion of cytokines, such as IL-2, IL-4, IL-5, IFN-γ, by enhancing the viability and expansion of effector T cells and memory T cells. Second, it reduces the immunosuppressive activity of regulatory T cells, thereby increases T cell activation. In the tumor microenvironment, activation of the immune system increases the expression of OX40, enhances the activation and proliferation of effector T cells, and inhibits regulatory T cells, which together contribute to a complex anti-tumor immune response. Currently, many clinical trials for anti-OX40 antibodies in cancer treatment are available on the Clinical Trials website.

However, there is still an emerging need for more novel anti-OX40 antibodies for the development of new cancer therapies.

### SUMMARY OF THE INVENTION

The present invention provides a novel anti-OX40 antibody that specifically binds to and activates OX40 for cancer treatment.

In one embodiment, the present invention relates to an anti-OX40 antibody or antigen-binding fragment thereof, which comprises: a heavy chain variable region with a heavy chain CDR1 domain (SEQ ID NO: 1), a heavy chain CDR2 domain (SEQ ID NO: 2), and a heavy chain CDR3 domain (SEQ ID NO: 3); and a light chain variable region with a light chain CDR1 domain (SEQ ID NO: 9), the light chain CDR2 domain (SEQ ID NO: 10), and the light chain CDR3 domain (SEQ ID NO: 11).

In another embodiment, the present invention provides an antibody-drug conjugate comprising the OX40 antibody or antigen-binding fragment thereof described herein and an additional therapeutic agents; preferably, the anti-OX40 antibody or antigen-binding fragment thereof is linked with the additional therapeutic agent by a linker.

In one aspect, the present invention provides a polynucleotide encoding the anti-OX40 antibody or antigen-binding fragment thereof described herein.

In another aspect, the present invention provides an expression vector comprising the polynucleotide described in the sequencing list.

In another aspect, the present invention provides a host cell comprising the polynucleotide described herein or the expression vector described herein.

In another embodiment, the present invention provides a method for producing the anti-OX40 antibody or antigen-binding fragment thereof described herein, which comprises culturing the host cell described herein under conditions suitable for the expression of the antibody or antigen-binding fragment thereof, and recovering the expressed antibody or antigen-binding fragment thereof from the culture medium.

In another embodiment, the present invention provides a pharmaceutical composition comprising the anti-OX40 antibody or antigen-binding fragment thereof described herein, or the antibody-drug conjugate described herein, or the polynucleotide described herein, or the expression vector described herein, and a pharmaceutically acceptable carrier.

In one aspect, the present invention provides the anti-OX40 antibody or antigen-binding fragment thereof described herein, the antibody-drug conjugate described herein, or the pharmaceutical composition described herein, used for the treatment of cancers.

In another aspect, the present invention provides a method for treating cancers, comprising administering to an individual in need thereof a therapeutically effective amount of the anti-OX40 antibody or antigen-binding fragment thereof described herein, the antibody-drug conjugate described herein, or the pharmaceutical composition described herein.

In another aspect, the present invention provides a use of the anti-OX40 antibody or antigen-binding fragment thereof described herein, or the antibody-drug conjugate described herein, or the pharmaceutical composition described herein in the manufacture of a medicament for treatment of cancers.

In another aspect, the present invention provides a use of the anti-OX40 antibody or antigen-binding fragment thereof described herein, or the antibody-drug conjugate described herein, or the pharmaceutical composition described herein in the manufacture of drugs for treatment of cancers.

In one embodiment, the present invention provides the anti-OX40 antibody or antigen-binding fragment thereof described herein, or the antibody-drug conjugate described herein, or the pharmaceutical composition described herein, used for one or more of the following: inhibiting Treg function (for example, inhibiting the suppressive function of Treg), killing OX40-expressing cells (for example, cells expressing high levels of OX40), increasing effector T cell function and/or increasing memory T cell function, reducing tumor immunity, enhancing T cell function and/or depleting cells expressing OX40.

In another embodiment, the present invention provides the anti-OX40 antibody or antigen-binding fragment thereof described herein, or the antibody-drug conjugate described herein, or the pharmaceutical composition described herein, used for manufacturing drugs with one or a combination of the following treatment mechanisms: inhibiting Treg function (for example, inhibiting the suppressive function of Treg), killing OX40-expressing cells (for example, cells expressing high levels of OX40), increasing effector T cell function and/or increasing memory T cell function, reducing tumor immunity, and enhancing T cell function and/or depleting cells expressing OX40.

In another aspect, the present invention provides a pharmaceutical composition comprising the anti-OX40 antibody or antigen-binding fragment thereof described herein, the antibody-drug conjugate described herein, or the pharmaceutical composition described herein, and one or more additional therapeutic agents.

In one aspect, the present invention provides a kit comprising the anti-OX40 antibody or antigen-binding fragment thereof described herein, or the antibody-drug conjugate described herein, or the pharmaceutical composition described herein, preferably further including an administration device.

In certain embodiments, the antibody of the invention contains one or more point mutations of its amino acid sequence, which are designed to improve the developmentability of the antibody. In a preferred embodiment, the one or more point mutations make the antibody more stable during the process of expression in host cells, purification in preparation and/or formulation, and/or administration to an individual. In another preferred embodiment, the one or more point mutations make the antibody less likely to aggregate during the preparation and/or formulation process. In some other embodiments, the present invention provides therapeutic antibodies that have minimized or reduced developmentability problems, for example, by replacing one or more amino acids of their sequence (e.g., in one or more of their CDRs) to remove or reduce the hydrophobicity and/or optimize the charge.

One embodiment of the present invention is a monoclonal antibody or antigen-binding fragment thereof, which can specifically bind to the epitope of OX40 corresponding to amino acid residues 56-74 (SEQ ID NO: 34: CSRSQNTVCRPCGPGFYN) of SEQ ID NO: 33.

Another embodiment of the present invention relates to a monoclonal antibody or antigen-binding fragment thereof capable of specifically binding to OX40, wherein the monoclonal antibody or antigen-binding fragment thereof does not interfere with the binding of OX40 with OX40 ligand.

Another embodiment of the present invention relates to said monoclonal antibody or its antigen-binding fragment, wherein the binding of the monoclonal antibody or its antigen-binding fragment does not interfere with the binding of OX40 with OX40 ligand in the state of trimeric or multimeric aggregation.

Another embodiment of the present invention is a monoclonal antibody or antigen-binding fragment thereof capable of specifically binding to OX40, wherein the binding of the antibody to OX40 and endogenous OX40 ligand together activate stimulatory signaling pathways.

Another embodiment of the present invention is the monoclonal antibody or antigen-binding fragment thereof according to any one of the above embodiments, wherein OX40 is human OX40.

Another embodiment of the present invention is the monoclonal antibody or antigen-binding fragment thereof in the above embodiments, wherein the epitope comprises SEQ ID NO:2.

Another embodiment of the present invention is the monoclonal antibody or antigen-binding fragment thereof in the above embodiments, wherein the antibody binds to OX40 with a K_{D} of about 1 nM to about 10 nM.

Another embodiment of the present invention is the monoclonal antibody or antigen-binding fragment thereof according to the above-mentioned embodiments, wherein the binding of the monoclonal antibody or antigen-binding fragment thereof to OX40 does not down-regulate the expression of OX40 or reduce the amount of OX40 present on the cell surface.

Another embodiment of the present invention is a method of treating cancers with said monoclonal antibody, wherein the cancer is a solid tumor, a non-solid tumor, or a type of cancer with OX40 expression on the cell surface of the tumor-infiltrating T cells.

Another embodiment of the present invention is a method for detecting OX40 in a sample through the binding of OX40 with the monoclonal antibody or antigen-binding fragment thereof.

Another embodiment of the present invention is a method for determining the level of OX40 in a subject, which comprises:
a) obtaining samples from said subject;
b) mixing the sample with the monoclonal antibody or antigen-binding fragment thereof of the present invention; and
c) determining the level of OX40 in the subject,
wherein the sample is a tissue sample, blood sample, or a tumor/cancer sample.

### BRIEF DESCRIPTION OF THE DRAWINGS

FIG. 1A shows the binding of HFB10-1E1hG1 to OX40 protein. The EC80 of HFB10-1E1hG1 is 0.71 nM.

FIG. 1B shows the binding of HFB10-1E1hG1 to the human OX40 protein expressed on the surface of 293T cells. The EC50 is 2 nM (Mean Fluorescence Intensity (MFI)).

FIG. 1C shows the binding of HFB10-1E1hG1 to the cynomolgus monkey OX40 protein expressed on the surface of 293T cells. The EC50 is 2.9 nM (MFI).

FIG. 1D shows the binding of HFB10-1E1hG1 to the mouse OX40 protein expressed on the surface of 293T cells. HFB10-1E1hG1 does not bind to the mouse OX40 protein (MFI) expressed on the surface of 293T cells.

FIG. 1E shows the binding of HFB10-1E1hG1 to the human CD40 protein expressed on the surface of 293T cells. HFB10-1E1hG1 does not bind to the human CD40 protein (MFI) expressed on the surface of 293T cells.

FIG. 1F shows the binding of the human OX40 protein expressed on the surface of 293T cells to each of HFB10-1E1hG1, Reference 1, Reference 2, Reference 3, and Reference 4. The EC50 of HFB10-1E1hG1 is 2.02 nM (MFI); the EC50 of Reference 1 is 2.67 nM (MFI); the EC50 of Reference 2 is 4.17 nM (MFI); the EC50 of Reference 3 is 1.92 nM (MFI); the EC50 of Reference 4 is 2.11 nM (MFI).

FIG. 1G shows the binding of the cynomolgus monkey OX40 protein expressed on the surface of 293T cells to each of HFB10-1E1hG1, Reference 1, Reference 2, Reference 3, and Reference 4. The EC50 of HFB10-1E1hG1 is 2.94 nM (MFI); the EC50 of Reference 1 is 2.91 nM (MFI); the EC50 of Reference 2 is 6.13 nM (MFI); the EC50 of Reference 3 is 2.57 nM (MFI); the EC50 of Reference 4 is 3.54 nM (MFI).

FIG. 2A shows the agonistic activity of HFB10-1E1hG1 in Jurkat reporter cells. When cross-linked with anti-human IgG, the EC50 of HFB10-1E1hG1 is 2.9 nM (MFI of GFP).

FIG. 2B shows the agonistic activity of HFB10-1E1hG1 in Jurkat reporter cells. The EC50 of HFB10-1E1hG1 is far greater when it is not cross-linked with anti-human IgG. Without cross-linking with anti-human IgG, the trimeric OX40L recombinant protein alone can activate the NF-kb signaling pathway when EC50 equals to 45 nM. MFI: measure of florescence (GFP) intensity.

FIG. 2C shows a synergetic agonistic effect of HFB10-1E1hG1 and OX40L in cross-linking with anti-human IgG. HFB10-1E1hG1 and OX40L together increased the MFI of GFP compared to the effect of each individual component acting alone.

FIG. 2D shows the agonistic activity of HFB10-1E1hG1 in primary CD4⁺ T cells. The agonistic activity of HFB10-1E1hG1 in primary CD4⁺ T cells was measured by the level of IL-2 secretion, and the obtained EC50 was 0.2 nM.

FIG. 2E shows the agonistic activity of HFB10-1E1hG1 in primary CD4⁺ T cells. HFB10-1E1hG1 and OX40L together have a synergetic agonistic effect in stimulating IL-2 secretion in primary CD4⁺ T cells.

FIG. 3 shows the pharmacokinetic test of HFB10-1E1hG1. The level of IgG decreased from 100⁺ g/mL to less than 10 μg/mL over a course of 100 hours post treatment.

FIG. 4A shows the anti-tumor efficacy of HFB10-1E1hG1 in vivo. Compared with the PBS control, HFB10-1E1hG1 significantly reduced tumor size.

FIG. 4B shows the anti-tumor efficacy of HFB10-1E1hG1 in vivo. HFB10-1E1hG1 did not show undesired side effects that caused significant body weight loss in mice.

FIG. 4C shows the in vivo anti-tumor efficacy (tumor size) of HFB10-1E1hG1 at 0.1 mg/kg, HFB10-1E1hG1 at 1 mg/kg, Reference 1 at 1 mg/kg, and Control at 10 mg/kg. HFB10-1E1hG1 treatment at 1 mg/kg was most effective in controlling the tumor size over a period of 15 days measured.

FIG. 4D shows the in vivo anti-tumor efficacy (body weight) of HFB10-1E1hG1 at 0.1 mg/kg, HFB10-1E1hG1 at 1 mg/kg, Reference 1 at 1 mg/kg, and Control at 10 mg/kg. HFB10-1E1hG1 did not show undesired side effects that led to significant body weight loss in mice.

FIG. 5A shows the effect of temperature on the stability of HFB10-1E1hG1. HFB10-1E1hG1 has good stability under different temperatures as suggested by the SDS-PAGE results.

FIG. 5B shows the effect of pH on the stability of HFB10-1E1hG1. HFB10-1E1hG1 showed good stability under different pH conditions as suggested by the SDS-PAGE results.

FIG. 5C shows the oxidative stress test of HFB10-1E1hG1. HFB10-1E1hG1 has good stability under different oxidative stress conditions as suggested by the SDS-PAGE results.

FIG. 5D shows the effect of freeze-thaw on the stability of HFB10-1E1hG1. HFB10-1E1hG1 has good stability under different freezing and thawing conditions as suggested by the SDS-PAGE results.

FIGS. 6A-6F depict the binding of HFB10-1E1hG1 to activated CD4⁺ T cells isolated from three different monkeys. Each curve represents a non-linear curve fitting using four-parameter model. Each dot on the curve represents an actual data point. #200107 (FIG. 6A: percentage of positive cells and FIG. 6B: MFI), #M20Z013009 (FIG. 6C: percentage of positive cells and FIG. 6D: MFI), and #M20Z025008 (FIG. 6E: percentage of positive cells and FIG. 6F: MFI).

FIG. 7 shows the mean antibody concentration versus time profiles of Bmk 1 (1 mg/kg and 10 mg/kg) and HFB10-1E1hG1 (1 mg/kg and 10 mg/kg) in wild-type (WT) mice. Symbols: average values of data points HFB10-1E1hG1 administration generated greater antibody concentration than Bmk 1 at both measured doses.

FIG. 8 shows the mean antibody concentration versus time profiles of Bmk 1 and HFB10-1E1hG1 in hOX40-KI mice. Symbols: average values of data points Administration of HFB10-1E1hG1 at 10 mg/kg has long-lasting effect (more than 1 μg/mL at 200 hours post administration) compared to the Bmk 1 (less than 0.1 μg/mL at about 70 hours post administration).

FIG. 9 shows the percentage of CD8⁺ T cells in CD45⁺ cells of tumor tissues.

FIG. 10 shows the percentage of Treg in CD4⁺ T cells of tumor tissues.

FIG. 11 shows Ki67 expression in CD4⁺ and CD8⁺ T cells of tumor tissues.

FIG. 12 shows PD-1 expression in CD4⁺ and CD8⁺ T cells of tumor tissues.

FIG. 13 shows HFB301001 as the epitope of the anti-OX40 antibody, which is mapped to the 3D crystal structure of OX40 and OX40L complex.

FIGS. 14A-14B show the agonistic activity of OX40 ligand measured using a cell-based bioluminescence assay. FIG. 14A shows the reporter gene activity of OX40 ligand alone; FIG. 14B shows the reporter gene activity of OX40 ligand in the presence of anti-OX40 antibody HFB301001, Benchmark 1 or Benchmark 2. The Y-axis shows relative luminescence (RLU).

FIGS. 15A-15B show the level of OX40 expression in CD4⁺ T cells after antibody treatment. FIG. 15A shows the percentage of OX40 positive CD4⁺ T cells isolated from human PBMC after treatment with different concentrations of anti-OX40 antibody as measured by flow cytometry. In a human OX40 (hOX40) knock-in MC-38 murine colorectal cancer model, the OX40 expression in CD4⁺ T cells was measured at 24 hours after the third treatment with 10 mg/kg anti-OX40 antibody (FIG. 15B). MFI represents the mean fluorescence intensity.

FIG. 16 shows the tumor size in the hOX40 knock-in MC-38 murine tumor model after treatment with an isotype control (10 mg/kg), an anti-OX40 antibody Benchmark 1 (1 mg/kg), or an anti-OX40 antibody HFB301001 (0.1 mg/kg and 1 mg/kg). The arrows indicate the treatment time points, and the error bar indicates the standard error. The significance is calculated by one-way ANOVA at the last time point (*: P<0.05, **: P<0.01).

FIG. 17 shows the survival percentage of human OX40 (hOX40) knock-in mice in the MC-38 murine colorectal cancer model after treatment with the anti-OX40 antibody HFB301001 (10 mg/kg, 1 mg/kg, 0.1 mg/kg), anti-OX40 antibody Benchmark 1 (10 mg/kg or 1 mg/kg), Control (10 mg/kg), or phosphate buffered saline (PBS). The significance was calculated by the logrank test (*: P<0.05, **: P<0.01).

FIGS. 18A-18E show the percentage of specific immune cell populations in the tumor microenvironment of hOX40 knock-in MC-38 mice colorectal cancer model after treatment with a Control (IgG1), an OX40 antibody Benchmark 1, or an anti-OX40 antibody HFB301001. FIG. 18A shows the percentage of CD3⁺CD4⁺Ki67⁺ T cells in the live CD45⁺CD3⁺CD4⁺ cells. FIG. 18B shows the percentage of CD3⁺CD8⁺Ki67⁺ T cells in the live CD45⁺CD3⁺CD8⁺ cells. FIG. 18C shows the percentage of CD3⁺CD4⁺PD-1⁺ T cells in the live CD45⁺CD3⁺CD4⁺ cells. FIG. 18D shows the percentage of CD3⁺CD8⁺PD-1⁺ T cells in the live CD45⁺CD3⁺CD8⁺ cells. FIG. 18E shows the percentage of CD3⁺CD4⁺CD25⁺Foxp³⁺ cells in the live CD45⁺CD3⁺CD4⁺ cells.

FIG. 19 shows luciferase signal from OX40 signaling reporter Jurkat T cells (NF-kB-Luc2/OX40 reporter Jurkat cells) after incubation with HFB10-1E1hG1, Benchmark 1 (Bmk1), or Benchmark 2 (Bmk2) antibody.

FIGS. 20A-20D show luciferase signal from OX40 reporter cells (NF-kB-Luc2/OX40 reporter Jurkat cells) stimulated by OX40 ligand (OXL40) in the presence of HFB10-1E1hG1 (FIG. 20A), isotype control (FIG. 20B), Benchmark 1 (FIG. 20C), and Benchmark 2 (FIG. 20D) antibody.

FIGS. 21A-21D show binding of HFB10-1E1hG1 to activated human CD4⁺CD3⁺ T cells. T cells were isolated from two donors, #190055 (FIG. 21A: percentage of positive cells; FIG. 21B: MFI) and #190061 (FIG. 21C: percentage of positive cells; FIG. 21D: MFI). Isotype control and Benchmark 1 antibody (Bmk1) are also tested in the experiment.

FIGS. 22A-22B show OX40 expression on primary CD4⁺ T cells following treatment with HFB10-1E1hG1. T cells were isolated from two different donors, #191223-B (FIG. 22A) and #190061 (FIG. 22B). OX40 expression on T cells was determined by staining with anti-OX40 antibody.

FIGS. 23A-23B show IL-2 levels after stimulation of human PBMCs with SEA and anti-OX40 antibodies, HFB10-1E1hG1 and Benchmark 1 (Bmk1). Human IL-2 was quantified in culture supernatants after 70 hrs of stimulation. PBMCs were stimulated with either 10 ng/mL (FIG. 23A) or 1 ng/mL (FIG. 23B) SEA. Each experimental condition was tested in duplicate. Symbols display the mean and error bars represent standard deviation (SD).

### DETAILED DESCRIPTION OF THE INVENTION

In one embodiment, the present invention relates to an anti-OX40 antibody or antigen-binding fragment thereof, which comprises: a heavy chain variable region with a heavy chain CDR1 domain (SEQ ID NO: 1), a heavy chain CDR2 domain (SEQ ID NO: 2), and a heavy chain CDR3 domain (SEQ ID NO: 3); and a light chain variable region with a light chain CDR1 domain (SEQ ID NO: 9), the light chain CDR2 domain (SEQ ID NO: 10), and the light chain CDR3 domain (SEQ ID NO: 11).

In another embodiment, the anti-OX40 antibody or antigen-binding fragment thereof described herein comprises a heavy chain variable region shown in SEQ ID NO: 4, or a heavy chain variable region having at least 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 4; and a light chain variable region shown in SEQ ID NO: 12, or a light chain variable region having at least 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 12.

In another embodiment, the anti-OX40 antibody or antigen-binding fragment thereof described herein, which further comprises a heavy chain constant region and a light chain constant region; preferably, the heavy chain constant region is SEQ ID NO: 5, or with at least 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 5; and/or preferably, the light chain constant region is SEQ ID NO: 13, or with at least 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 13.

In another embodiment, the anti-OX40 antibody or antigen-binding fragment thereof described herein further comprises a heavy chain signal peptide linked to the heavy chain variable region and/or a light chain signal peptide linked to the light chain variable region; preferably, the heavy chain signal peptide is SEQ ID NO: 6 or with at least 80%, 81%, 82%, 83%, 84%, 85% 86%, 87%, 88%, 89%, 90%, 91%, 92%, 93%, 94%, 95%, 96%, 97%, 98%, 99% or 100% identity with SEQ ID NO: 6; and/or preferably, the light chain signal peptide SEQ ID NO: 14, or with at least 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 14.

In another embodiment, the anti-OX40 antibody or antigen-binding fragment thereof described herein is an IgG antibody or antigen-binding fragment thereof, or preferably an IgG1 antibody or antigen-binding fragment thereof.

In another embodiment, the anti-OX40 antibody or antigen-binding fragment thereof described herein is a monoclonal antibody or antigen-binding fragment thereof.

In another embodiment, the anti-OX40 antigen-binding fragment described herein is Fab, Fab′, F(ab′)2, Fv, scFv, or sdAb.

In certain embodiments, the isolated monoclonal antibody, or antigen-binding fragment thereof, specifically binds to OX40 (e.g., human OX40) at an epitope comprising, consisting essentially of, or consisting of SEQ ID NO: 34, as an OX40 agonist, and does not substantially affect OX40-OX40L interaction/binding.

In certain embodiments, the isolated monoclonal antibody, or antigen-binding fragment thereof induces NF-kB-mediated OX40 signaling in the presence or absence of OX40L.

In certain embodiments, the isolated monoclonal antibody, or antigen-binding fragment thereof does not inhibit, or enhances, (endogenous) OX40L-induced OX40 signaling (e.g., NF-kB signaling), optionally, OX40 signaling is induced by OX40L at a concentration of at least about 50 nM, 60 nM, 70 nM, 80 nM, 90 nM or more.

In certain embodiments, the isolated monoclonal antibody, or antigen-binding fragment thereof binds to activated primary (human or cynomolgus monkey) CD4⁺CD3⁺ T cells with an EC50 value of about 0.01-5 nM, 0.1-0.3 nM, or 0.5-1.5 nM (e.g., 0.65 or 1.36 nM).

In certain embodiments, the isolated monoclonal antibody, or antigen-binding fragment thereof increases total surface level OX40 expression on active (e.g., CD3/CD28-stimulated) peripheral human CD4⁺ T cells in a dose-dependent manner (e.g., with a maximum OX40 expression stimulated by about 1 nM of said antibody or fragment thereof).

In certain embodiments, the isolated monoclonal antibody, or antigen-binding fragment thereof dose-dependently augment T-cell activation.

In certain embodiments, the isolated monoclonal antibody, or antigen-binding fragment thereof increases cytokine (e.g., IL-2) production in activated human PBMCs; optionally, said human PBMCs are stimulated by Staphylococcus aureus enterotoxin A (SEA) at 1 or 10 ng/mL.

In certain embodiments, the isolated monoclonal antibody, or antigen-binding fragment thereof dose-dependently induce CD16-mediated ADCC in effector cells in the presence of OX-40-expressing target cells, e.g., with an EC50 of about 0.3-1.2 nM at effector to target cell (E:T) ratios of 12:1 to 1:1.

In certain embodiments, the isolated monoclonal antibody, or antigen-binding fragment thereof does not induce significant cytokine release in primary PBMCs at up to 300 μg/mL.

In certain embodiments, the isolated monoclonal antibody, or antigen-binding fragment thereof does not induce significant toxicity in mouse at a maximum tolerable dose of at least about 100 mg/kg.

In one aspect, the present invention relates to an antibody-drug conjugate comprising the OX40 antibody or antigen-binding fragment thereof described herein and some additional therapeutic agents; preferably, the anti-OX40 antibody or antigen-binding fragment thereof is linked with the additional therapeutic agent by a linker.

In one aspect, the present invention comprises a polynucleotide encoding the anti-OX40 antibody or antigen-binding fragment thereof described herein.

In one embodiment, the polynucleotide described herein comprises a heavy chain variable region polynucleotide coding sequence of SEQ ID NO: 20 and/or a light chain variable region polynucleotide coding sequence of SEQ ID NO: 28; preferably, the polynucleotide further comprises a heavy chain constant region polynucleotide coding sequence of SEQ ID NO: 21 and/or a light chain constant region polynucleotide coding sequence of SEQ ID NO: 29.

In another embodiment, the present invention relates to an expression vector comprising the polynucleotide described herein.

In another embodiment, the present invention provides a host cell comprising the polynucleotide described herein or the expression vector described herein.

In one aspect, the present invention provides a method for producing the anti-OX40 antibody or antigen-binding fragment thereof described herein, which comprises culturing the host cell described herein under conditions suitable for the expression of the antibody or antigen-binding fragment thereof, and recovering the expressed antibody or antigen-binding fragment thereof from the culture medium.

In another aspect, the present invention provides a pharmaceutical composition comprising the anti-OX40 antibody or antigen-binding fragment thereof described herein, the antibody-drug conjugate described herein, the polynucleotide described herein, or the expression vector described herein, and a pharmaceutically acceptable carrier.

In another aspect, the anti-OX40 antibody or antigen-binding fragment thereof described herein, the antibody-drug conjugate described herein, or the pharmaceutical composition described herein is used to treat cancers. In an embodiment, the cancer is selected from a group consisting of: squamous cell carcinoma (e.g., epithelial squamous cell carcinoma), lung cancer (including small cell lung cancer, non-small cell lung cancer, adenocarcinoma of the lung, and squamous cell carcinoma of the lung), peritoneal cancer, hepatocellular carcinoma, gastric cancer (including gastrointestinal cancer and gastrointestinal stromal cancer), pancreatic cancer, glioblastoma, cervical cancer, ovarian cancer, liver cancer, bladder cancer, urethral cancer, liver tumor, breast cancer, colon cancer, rectal cancer, colorectal cancer, endometrial or uterine cancer, salivary gland cancer, kidney cancer, prostate cancer, vulvar cancer, thyroid cancer, liver cancer, anal cancer, penile cancer, melanoma, superficial spreading melanoma, malignant lentigines melanoma, acral melanoma, nodular melanoma, multiple myeloma and B-cell lymphoma, chronic lymphocytic leukemia (CLL), acute lymphoblastic leukemia (ALL), hairy cell leukemia, chronic myeloblastic leukemia, and post-transplant lymphoproliferative disorder (PTLD), and abnormal vascular proliferation related to scar nevus, edema (for example, related to brain tumors) and Meigs syndrome, brain tumors and brain cancers, head and neck cancers, and related metastases.

In another aspect, the present invention provides a method of treating a subject having cancers, comprising administering to the subject a therapeutically effective amount of the anti-OX40 antibody or antigen-binding fragment thereof described herein, the antibody-drug conjugate described herein, or the pharmaceutical composition described herein, thereby treating said subject. In one embodiment, the cancer is selected from a group consisting of: squamous cell carcinoma (e.g., epithelial squamous cell carcinoma), lung cancer (including small cell lung cancer, non-small cell lung cancer, adenocarcinoma of the lung, and squamous cell carcinoma of the lung), peritoneal cancer, hepatocellular carcinoma, gastric cancer (including gastrointestinal cancer and gastrointestinal stromal cancer), pancreatic cancer, glioblastoma, cervical cancer, ovarian cancer, liver cancer, bladder cancer, urethral cancer, liver tumor, breast cancer, colon cancer, rectal cancer, colorectal cancer, endometrial or uterine cancer, salivary gland cancer, kidney cancer, prostate cancer, vulvar cancer, thyroid cancer, liver cancer, anal cancer, penile cancer, melanoma, superficial spreading melanoma, malignant lentigines melanoma, acral melanoma, nodular melanoma, multiple myeloma and B-cell lymphoma, chronic lymphocytic leukemia (CLL), acute lymphoblastic leukemia (ALL), hairy cell leukemia, chronic myeloblastic leukemia, and post-transplant lymphoproliferative disorder (PTLD), and abnormal vascular proliferation related to scar nevus, edema (for example, related to brain tumors) and Meigs syndrome, brain tumors and brain cancers, head and neck cancers, and related metastases.

In one aspect, the present invention provides a use of the anti-OX40 antibody or antigen-binding fragment thereof described herein, or the antibody-drug conjugate described herein, or the pharmaceutical composition described herein in the manufacture of another pharmaceutical composition for the treatment of cancers. In one embodiment, the cancer is selected from a group consisting of: squamous cell carcinoma (e.g., epithelial squamous cell carcinoma), lung cancer (including small cell lung cancer, non-small cell lung cancer, adenocarcinoma of the lung, and squamous cell carcinoma of the lung), peritoneal cancer, hepatocellular carcinoma, gastric cancer (including gastrointestinal cancer and gastrointestinal stromal cancer), pancreatic cancer, glioblastoma, cervical cancer, ovarian cancer, liver cancer, bladder cancer, urethral cancer, liver tumor, breast cancer, colon cancer, rectal cancer, colorectal cancer, endometrial or uterine cancer, salivary gland cancer, kidney cancer, prostate cancer, vulvar cancer, thyroid cancer, liver cancer, anal cancer, penile cancer, melanoma, superficial spreading melanoma, malignant lentigines melanoma, acral melanoma, nodular melanoma, multiple myeloma and B-cell lymphoma, chronic lymphocytic leukemia (CLL), acute lymphoblastic leukemia (ALL), hairy cell leukemia, chronic myeloblastic leukemia, and post-transplant lymphoproliferative disorder (PTLD), and abnormal vascular proliferation related to scar nevus, edema (for example, related to brain tumors) and Meigs syndrome, brain tumors and brain cancers, head and neck cancers, and related metastases.

In one aspect, the present invention relates to the anti-OX40 antibody or antigen-binding fragment thereof described herein, or the antibody-drug conjugate described herein, or the pharmaceutical composition described herein, used for one or more of the following: inhibiting Treg function (for example, inhibiting the suppressive function of Treg), killing OX40-expressing cells (for example, cells expressing high levels of OX40), increasing effector T cell function and/or increasing memory T cell function, reducing tumor immunity, and enhancing T cell function and/or depleting cells expressing OX40.

In one aspect, the present invention provides a use of the anti-OX40 antibody or antigen-binding fragment thereof described herein, the antibody-drug conjugate described herein, or the pharmaceutical composition described herein in the manufacture of another pharmaceutical composition for the treatment of one or more of the following: inhibiting Treg function (for example, inhibiting the suppressive function of Treg), killing OX40-expressing cells (for example, cells expressing high levels of OX40), increasing effector T cell function and/or increasing memory T cell function, reducing tumor immunity, enhancing T cell function, and/or depleting OX40 expressing cells.

In one aspect, the present invention provides a pharmaceutical combination comprising the anti-OX40 antibody or antigen-binding fragment thereof described herein, or the antibody-drug conjugate described herein, or the pharmaceutical composition described herein, and one or more additional therapeutic agents.

In one aspect, the present invention provides a kit comprising the anti-OX40 antibody or antigen-binding fragment thereof described herein, the antibody-drug conjugate described herein, or the pharmaceutical composition described herein, preferably further including an administration device.

Certain embodiments of the present invention relate to OX40 agonistic antibodies that are specific to a unique OX40 epitope, thus result in little or no down-regulation of OX-40 receptor compared to other OX40 agonistic antibodies. Compared to other known agonistic antibodies in the market, the disclosed agonistic antibodies may display better binding kinetics.

The disclosed invention or related applications could be enabled by people skilled in the art to understand and combine one, some, or all of the information disclosed in this invention. The following disclosure provides a detailed description of current invention and its related applications.

Two polynucleotide or polypeptide sequences are said to be “identical” if the sequence of the nucleotides or amino acids in the two sequences is the same when there is a perfect match of the alignment. Comparisons between two sequences are typically performed by comparing the sequences over a comparison window to identify and compare sequence similarity over some local regions. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Complete sequence alignment may be performed using the default parameters of the MegAlign® program in the Lasergene® suite of bioinformatics software (DNASTAR®, Inc., Madison, WI). This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington DC Vol. 5, Sup. 3, pp. 345-358; Hein J., 1990, Unified Approach to Alignment and Phylogenes pp. 626-645 Methods in Enzymology vol. 183, Academic Press, Inc., San Diego, CA; Higgins, D. G. and Sharp, P. M., 1989, CABIOS 5: 151-153; Myers, E. W. and Muller W., 1988, CABIOS 4: 11-17; Robinson, E. D., 1971, Comb. Theor. 1 1:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

In some embodiments, the “percentage of sequence identity” is determined by the comparison of a complete sequence alignment over at least 20 contiguous nucleic acids or amino acid residues, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which do not include additions or deletions). The percentage is calculated by dividing the number of matched positions by the total number of positions in the reference sequence (i.e., the window size) and multiplying the results by 100 to yield the percentage of sequence identity.

Alternatively, a variant could be substantially homologous to a native gene, or a portion or complement thereof. Such polynucleotide variants are capable of hybridizing with a naturally occurring DNA sequence encoding a native antibody (or a complementary sequence) under moderately stringent conditions.

Said “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice with 2×, 0.5× and 0.2×SSC containing 0.1% SDS at 65° C. for 20 minutes.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ a denaturing agent during hybridization, such as formamide, for example, 50% (v/v) of formamide with 0.1% bovine serum albumin/0.1% Ficoll/0.1% polyvinylpyrrolidone/50 mM sodium phosphate buffer at pH 6.5 and 50% of 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 Mg/mL), 0.1% SDS, and 10% dextran sulfate at 42° C., and wash at 42° C. in 0.2×SSC (sodium chloride/sodium citrate) and 50% formamide at 55° C., followed by a high-stringency wash with 0.1×SSC containing EDTA at 55° C. People skilled in the art would optimize the experimental conditions, such as, temperature and ionic strength as necessary to accommodate variables such as probe length and the like.

It will be appreciated by those of ordinary skill in the art that, as a result of the degeneracy of the genetic code, there are many nucleotide sequences that may encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this disclosure can be obtained using chemical synthesis, recombinant methods, or PCR. Methods of chemical polynucleotide synthesis are well known in the art and need not to be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Said host cells are transformed by introducing an exogenous polynucleotide by direct uptake, endocytosis, transfection, F-mating, or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences.

RNA can be obtained by using the isolated DNA in an appropriate vector and inserting it into a suitable host cell. When the cell replicates and the DNA is transcribed into RNA, the RNA can then be isolated using methods well known to those of skill in the art.

Suitable cloning and expression vectors can include a variety of components, such as promoter, enhancer, and other transcriptional regulatory sequences. The vector may also be constructed to allow for subsequent cloning of an antibody variable domain into different vectors.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry gene markers that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, Co1E1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the disclosure. The implied expression vector must be able to replicate in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest and/or the polynucleotides themselves can be introduced into the host cell by any appropriate means, including electroporation, transfection employing calcium chloride, rubidium chloride, calcium phosphate, DEAE-dextran, or other substances such as microprojectile bombardment; lipofection; and infection (e.g., where the vector is an infectious agent such as vaccinia virus). The choice of introducing vectors or polynucleotides will often depend on features of the host cell.

The antibodies of the present invention include human antibodies prepared, expressed, produced or isolated by recombinant methods, such as antibodies expressed using recombinant expression vectors transfected into host cells (further described in the following section II), antibodies isolated from recombinant combinatorial human antibody libraries (further described in section III below), antibodies isolated from human immunoglobulin gene transgenic animals (e.g. mice) see e.g. (Taylor, L D et al. (1992) Nucl. Acids Res. 20: 6287-6295), or relate to antibodies prepared, expressed, produced or isolated by any other method involving the splicing of human immunoglobulin gene sequences into other DNA sequences. Such recombinant human antibodies have variable and constant regions derived from human germline immunoglobulin sequences (See Kabat, E A, et al. (1991) Sequences of Proteins of Immunological Interest, Fifth Edition, US Department of Health and Human Services, NIH Publication No. 91-3242).

The antibody or antibody portion of the present invention can be prepared by recombinantly expressing immunoglobulin light chain genes and heavy chain genes in host cells. In order to recombinantly express the antibody, a host cell is transfected with one or more recombinant expression vectors carrying DNA fragments encoding the immunoglobulin light and heavy chains of the antibody, so that the light and heavy chains are expressed in the host cell, and preferably secreted into a medium in which the host cell is cultured, from which the antibody can be recovered. Standard recombinant DNA methodology is used to obtain antibody heavy chain genes and antibody light chain genes, introduce these genes into a recombinant expression vector, and then introduce the vector into host cells. The standard methods are, for example, the methods described in the following literature: Sambrook, Fritsch and Maniatis (editors), Molecular Cloning; A Laboratory Manual, Second Edition, Cold Spring Harbor, NY, (1989), Ausubel, F M, etc. (editors) Current Protocols in Molecular Biology, Greene Publishing Associates, (1989) and Boss Et al. U.S. Pat. No. 4,816,397.

The antibody, or antigen-binding fragment thereof, may be made recombinantly using a suitable host cell. A polynucleotide encoding the antibody or antigen-binding fragment thereof can be cloned into an expression vector, which can then be introduced into a host cell, such as E. coli cell, a yeast cell, an insect cell, a simian COS cell, a Chinese hamster ovary (CHO) cell, or a myeloma cell where the cell does not otherwise produce an immunoglobulin protein to obtain the synthesis of an antibody in the recombinant host cell. Preferred host cells include a CHO cell, a human embryonic kidney (HEK) 293 cell, or an Sp2.0 cell, among many cells well-known in the art.

An antibody fragment can be produced by proteolytic or other degradation of a full-length antibody, recombinant methods, or chemical synthesis. A polypeptide fragment of an antibody, especially shorter polypeptides up to about 50 amino acids, can be conveniently made by chemical synthesis. Methods of chemical synthesis for proteins and peptides are known in the art and are commercially available.

The antibody, or antigen-binding fragment thereof, of the invention may be affinity matured. For example, an affinity matured antibody can be produced by procedures known in the art (Marks et al., 1992, Bio/Technology, 10:779-783; Barbas et al., 1994, Proc Nat. Acad. Sci, USA 91:3809-3813; Schier et al., 1995, Gene, 169: 147-155; Yelton et al., 1995, J. Immunol., 155:1994-2004; Jackson et al., 1995, J. Immunol., 154(7):3310-9; Hawkins et al., 1992, J. Mol. Biol., 226:889-896; and WO2004/058184).

### Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, insertions into, and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, the antibody of the invention contains one or more point mutations of its amino acid sequence, which are designed to improve the developability of the antibody. For example, Raybould et al., “Five computational developability guidelines for therapeutic antibody profiling,” PNAS, Mar. 5, 2019, 116 (10) 4025-4030 described its Therapeutic Antibody Analysis Tool (TAP), which is a calculation tool established for downloadable homology models of variable domain sequences, tested against five developability guidelines, and reporting of potential sequence liabilities and canonical form. The author further provides TAP, which is freely available at opig.stats.ox.ac.uk/webapps/sabdab-sabpred/TAP.php. In addition to obtaining the desired affinity for the antigen, there are many obstacles to the development of therapeutic monoclonal antibodies, which include innate immunogenicity, chemical and conformational instability, self-association, high viscosity, multispecificity, and poor expression. For example, high levels of hydrophobicity (especially in the highly variable complementarity determining regions (CDR)) have repeatedly been implicated in aggregation, viscosity, and multispecificity. The asymmetry in the net charge of the heavy and light chain variable domains is also related to self-association and viscosity at high concentrations. The positively and negatively charged patches in the CDR are related to high clearance rates and poor expression levels. Product heterogeneity (for example, by oxidation, isomerization, or glycosylation) is usually caused by specific sequence motifs that are prone to post-translational or co-translational modifications. Calculation tools can be used to facilitate the identification of sequence liabilities. Warszawski also described methods for optimizing antibody affinity and stability through the automated design of the variable light chain-heavy chain interface. Warszawski et al. (2019) Optimizing antibody affinity and stability by the automated design of the variable light-heavy chain interfaces, PLoS Comput Biol 15 (8): e1007207, https://doi.org/10.1371/journal.pcbi.1007207. Other methods can be used to identify potential developability problems of candidate antibodies, and in a preferred embodiment of the present invention, one or more point mutations can be introduced into candidate antibodies by conventional methods to solve such problems, thereby obtaining optimized therapeutic antibodies of the present invention.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table A under the heading of “preferred substitutions.” More substantial changes are provided in Table A under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE A

| Original | Exemplary | Preferred |
|---|---|---|
| residue | Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to their common side-chain characteristics:
(1) hydrophobic: norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated, and the variant antibodies will be displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207: 179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178: 1-37 (O'Brien et al., edit, Human Press, Totowa, NJ, (2001).) In some embodiments of affinity maturation, diversity is introduced into the variable genes chosen for maturation by a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered or contains no more than one, two, or three amino acid substitutions.

A useful method for the identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as arg, asp, his, lys, and glu) are identified and replaced by a neutral or negatively charged amino acid (e.g., alanine or polyalanine) to determine whether the antibody-antigen interaction is affected. Further substitutions may be introduced at the amino acid locations demonstrating functional sensitivity to the initial substitutions. Alternatively, or additionally, a crystal structure of an antigen-antibody complex can be used to identify the contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide that increases the serum half-life of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create the antibody variants with certain improved properties.

In one embodiment, the provided antibody variants have a carbohydrate structure that lacks direct or indirect fucose attachment ability to an Fc region. For example, the amount of fucose in such antibody may range from 1% to 80%, 1% to 65%, 5% to 65%, or 20% to 40%. The amount of fucose is determined by the ratio of the average amount of fucose within the sugar chain at Asn297 to the sum of all glycostructures attached to Asn 297 (e. g. complex, hybrid and high mannose structures) as measured by MALDI-TOF mass spectrometry described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US2004/0093621 (Kyowa Hakko Kogyo Co., Ltd). Examples of publications related to “defucosylated” or “fucose-deficient” antibody variants include: US 2003/0157108; WO 2000/61739; WO2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. Mol. Biol. 336: 1239-1249 (2004); Yamane-Ohnuki et al. Biotech. Bioeng. 87: 614 (2004). Examples of cell lines capable of producing defucosylated antibodies include Led 3 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

c) Fc Region Variants In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) with one or more amino acid modifications (e.g. a substitution).

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. The expression of FcR on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82: 1499-1502 (1985); 5,821,337 (see Bruggemann, M. et al., Exp. Med. 166: 1351-1361 (1987)). Alternatively, non-radioactive assays methods may be employed (see, for example, ACT I™ nonradioactive cytotoxicity assay for flow cytometry (CellTechnology, Inc. Mountain View, CA; and CytoTox 96 non-radioactive cytotoxicity assay (Promega, Madison, WI))). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al., J. Immunol. Methods 202: 163 (1996); Cragg, M. S. et al., Blood 101: 1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12): 1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include the substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding capacity to FcRs are described. (See, e.g., U.S. Pat. No. 6,737,056; WO 2004/056312, and Shields et al., Biol. Chem. 9(2): 6591-6604 (2001)).

In certain embodiments, an antibody variant comprises an Fc region with one or more amino acid substitutions that improve ADCC, e.g., substitutions at positions 298, 333, and/or 334 of the Fc region (EU numbering of residues).

In some embodiments, alterations are made in the Fc region that result in altered (i.e., either improved or diminished) Clq binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn) were described in US2005/0014934A1 (Hinton et al.). The FcRn is responsible for the transfer of maternal IgGs to the fetus (Guyer et al., Immunol. 117:587 (1976) and Kim et al., Immunol. 24:249 (1994)). Those antibodies comprise an Fc region with one or more substitutions that improve the binding capacity of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also examples of Fc region variants in Duncan & Winter, Nature 322:738-40 (1988); U.S. Pat. Nos. 5,648,260; 5,624,821; and WO 94/29351.

### d) Cysteine Engineered Antibody Variants

In certain embodiments, it may be desirable to create the cysteine engineered antibodies, e.g., “thioMAb,” in which one or more residues of said antibodies are substituted with cysteine residues. In one embodiment, the substituted residues exist at some accessible sites of the antibody. By substituting those residues with cysteine, reactive thiol groups are thereby positioned at the accessible sites of the antibody and may be used to conjugate the antibody to other moieties, such as drug moieties or linker-drug moieties, to create an immunoconjugate, as described further herein. In another embodiment, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for antibody derivatization include, but not limited to, water soluble polymers. Non-limiting examples of water soluble polymers include, but not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(η-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymers are attached, they can be the same or different molecules. In general, the number and/or type of polymers used for derivatization can be determined based on considerations including, but not limited to, the particular properties or functions of the antibody to be improved, whether the antibody derivative will be used in a therapy with defined conditions, etc.

In one embodiment, conjugates of an antibody and nonproteinaceous moiety are provided, wherein the nonproteinaceous moiety may be selectively heated by exposure to radiation. In another embodiment, the nonproteinaceous moiety is a carbon nanotube (Kam et al., Proc. Natl. Acad. Sci. USA 102: 11600-11605 (2005)). The radiation may be of any wavelengths including, but not limited to, wavelengths that do not damage nomal cells, but could raise the temperature of the antibody-nonproteinaceous moiety to a temperature that kills cells near the antibody-nonproteinaceous moiety.

### Assays

Anti-OX40 antibodies provided herein may be identified, screened, or characterized for their physical/chemical properties and/or biological activities by various assays known in the art.

### 1. Binding Assays and Other Assays

In one aspect, the antigen binding activity of the antibodies described herein is measured by known methods such as ELISA, Western blot, etc. OX40 binding capacity may be determined using methods known in the art, and the exemplary methods are disclosed herein. In one embodiment, an exemplary radioimmunoassay is used in measuring the antigen-antibody binding. OX40 antibody is iodinated, and competition reaction mixtures containing a fixed concentration of iodinated antibody and decreasing concentrations of serially diluted, unlabeled OZ X40 antibody are prepared. Cells expressing OX40 (e.g., BT474 cells stably transfected with human OX40) are added to the reaction mixture. Following an incubation, the free iodinated OX40 antibodies are washed away. Cell-bound iodinated OX40 antibodies are measured e.g., by counting radioactivity associated with cells, and binding affinity determined using standard methods. In another embodiment, the OX40 antibody binding capacity to the OX40 surface-expressed cells (e.g., a subset of T cells) is measured with flow cytometry. Peripheral white blood cells are obtained (e.g., from human, cynomolgus monkey, rat or mouse), and cells are blocked with serum. Labeled OX40 antibody is added in a serial dilution, and T cells are also stained to identify the T cell subsets (using methods known in the art). Following incubation and washing, the cells are sorted by flow cytometry, and data are analyzed using methods well known in the art. In another embodiment, OX40 binding capacity may be analyzed using surface plasmon resonance. An exemplary surface plasmon resonance method is exemplified.

In another aspect, competition assays may be used to identify an OX40 binding antibody that competes with any one of the anti-OX40 antibodies disclosed herein. In certain embodiments, such a competing antibody binds to the same epitope (e.g., a linear or a conformational epitope) as the anti-OX40 antibodies disclosed herein. Detailed exemplary methods for antibody binding epitope mapping are provided in Morris (1996) “Epitope Mapping Protocols,” in Methods in Molecular Biology vol. 66 (Humana Press, Totowa, NJ). A competition assay is exemplified.

In an exemplary competitive ELISA assay, immobilized OX40 is incubated in a solution comprising a first labeled anti-OX40 antibody (e.g., mab 1A7.gr.1, mab 3C8.gr5) and a second unlabeled antibody that is being tested for its ability to compete with the first anti-OX40 antibody. The second antibody may be present in a hybridoma supernatant. As a control, the immobilized OX40 is incubated in a solution comprising the first labeled anti-OX40 antibody but not the second unlabeled antibody. After incubation and under appropriate conditions that allow OX40 to bind the first labeled anti-OX40 antibody, excess unbound antibodies are removed, and the amount of signal associated with the immobilized OX40 is measured. If the detected signal is significantly lower in the tested sample than the control, it suggests that the second antibody competes with the first antibody in binding to the immobilized OX40. See Harlow and Lane (1988) Antibodies: A Laboratory Manual ch. 14 (Cold Spring Harbor Laboratory, Cold Spring Harbor, NY).

### 2. Activity Assays

In one embodiment, a method for detecting anti-OX40 antibodies with biological activities is provided. Biological activities may include, e.g., binding OX40 (e.g., binding human and/or cynomolgus OX40), increasing OX40-mediated signal transduction (e.g., increasing NFkB-mediated transcription), depleting cells that express human OX40 (e.g., T cells), depleting cells that express human OX40 by ADCC and/or phagocytosis, enhancing T effector cell function (e.g., CD4⁺ effector T cells), e.g., by increasing effector T cell proliferation and/or increasing cytokine production of effector T cells (e.g., γ interferon), enhancing memory T cell function (e.g., CD4⁺ memory T cell), e.g., by increasing memory T cell proliferation and/or increasing cytokine production by memory T cells (e.g., γ interferon), inhibiting regulatory T cell function, e.g., by decreasing Treg suppression of effector T cell function (e.g., CD4⁺ effector T cell function), and binding human effector cells. Antibodies having such biological activity both in vivo and/or in vitro are also provided herein.

In certain embodiments, an antibody of the invention is tested for such biological activity.

Methods known in the art for the measurement of T cell costimulation are exemplified herein. For example, T cells (e.g., memory or effector T cells) may be obtained from peripheral white blood cells (e.g., isolated from human whole blood using Ficoll gradient centrifugation). Memory T cells (e.g., CD4⁺ memory T cells) or effector T cells (e.g. CD4⁺ Teff cells) may be isolated from PBMC using methods known in the art. For example, the Miltenyi CD4⁺ memory T cell isolation kit or Miltenyi naive CD4⁺ T cell isolation kit may be used. Isolated T cells are cultured in the presence of antigen presenting cells (e.g., irradiated L cells that express CD32 and CD80), and activated by supplementation of anti-CD3 antibody in the presence or absence of OX40 agonist antibody. The Effect of agonist OX40 antibody on T cell proliferation may be measured by using methods well known in the art. For example, the CellTiter Glo kit (Promega) may be used, and results read on a Multimode Plate Reader (Perkin Elmer). Alternatively, the effect of agonist OX40 antibody on T cell function may also be determined by analysis of cytokines produced by the T cell. In one embodiment, production of interferon γ by CD4⁺ T cells is determined, e.g., by the measurement of interferon γ in cell culture supernatant. Methods for measuring interferon γ are well-known in the art.

Methods known in the art for the measurement of Treg cell functions are exemplified herein. In one example, the ability of Treg to suppress effector T cell proliferation is assayed. T cells are isolated from human whole blood using methods known in the art (e.g., isolating memory T cells or naive T cells). Purified CD4⁺ naive T cells are labeled (e.g., with CFSE), and purified Treg cells are labeled with a different reagent. Irradiated antigen presenting cells (e.g., L cells expressing CD32 and CD80) are co-cultured with the labeled purified naive CD4⁺ T cells and purified Tregs. The co-cultures are activated using anti-CD3 antibody and tested in the presence or absence of agonist OMO antibody. Following a suitable time (e.g., 6 days of coculture), level of CD4⁺ naive T cell proliferation is tracked by dye dilution in reduced label staining (e.g., reduced staining of CFSE label) using FACS analysis.

Methods known in the art for the measurement of OX40 signaling are exemplified herein. In one embodiment, transgenic cells expressing OX40 and its reporter gene (including NFkB promoter fused to a reporter gene (e.g., beta luciferase) are generated. Increased NFkB transcription resulted from the increased OX40 agonist antibody could be measured using a reporter gene assay.

Phagocytosis may be measured, for instance, by using monocyte-derived macrophages, or U937 cells (a human histiocytic lymphoma cells line with the morphology and characteristics of mature macrophages). OX40 expressing cells are added to the monocyte-derived macrophages or U937 cells in the presence or absence of anti-OX40 agonist antibody. Following culturing of the cells for a suitable period of time, the percentage of phagocytosis is determined by the ratio of the number of cells with double-staining markers of 1) the macrophage or U937 cell and 2) the OX40 expressing cell to the total number of cells with OX40 expressing marker (e.g., GFP). Analysis may be done by either flow cytometry or fluorescent microscopy.

Methods for the measure of ADCC are well known in the art and exemplified in the definition section. In some embodiments, the level of OX40 in OX40-expressing cells is characterized by ADCC assay. The cell may be stained with a labeled anti-OX40 antibody (e.g., PE labeled), then the level of fluorescence determined using flow cytometry, and results are presented as median fluorescence intensity (MFI). In another embodiment, ADCC may be analyzed by CellTiter Glo assay kit and cell viability/cytotoxicity may be determined by chemoluminescence.

The binding affinities of various antibodies to Fc γ RIA, Fc γ RIIA, Fc γ RIIB, and Fc γ RIIIA (F158 and V158) may be measured by ELISA-based ligand-binding assays using the respective recombinant Fcγ receptors. Purified human Fcγ receptors are expressed as fusion proteins containing the extracellular domain of the receptor γ chain linked to a Gly/6×His/glutathione S-transferase (GST) polypeptide tag at the C-terminus. The binding affinities of antibodies to those human Fcγ receptors are evaluated as follows. For the low-affinity receptors, i.e. Fc γRIIA (CD32A), Fc 7RIIB(CD32B), and Fe 7RIIIA(CD16), and the two allotypes of Fe 7RIIIA (CD 16), F-158 and V-158, antibodies may be tested as multimers by cross-linking with a F(ab′)2 fragment of goat anti-human kappa chain (ICN Biomedical; Irvine, CA) at an approximate molar ratio of 1:3 in antibody: cross-linking F(ab′)2. Plates are coated with an anti-GST antibody (Genentech) and blocked with bovine serum albumin (BSA). After washing with phosphate-buffered saline (PBS) containing 0.05% Tween-20 using an ELx405™ plate washer (Biotek Instruments; Winooski, VT), Fcγ receptors are added to the plate at 25 ng/well and incubated at room temperature for 1 hour. After washing, a serial dilutions of test antibodies are added as multimeric complexes and the plates were incubated at room temperature for 2 hours. Following plate washing to remove unbound antibodies, the antibodies bound to the Fcγ receptor are detected with horseradish peroxidase (HRP)-conjugated F(ab′)2 fragment of goat anti-human F(ab′)2 (Jackson ImmunoResearch Laboratories; West Grove, PA) followed by the addition of substrate, tetramethylbenzidine (TMB) (Kirkegaard & Perry Laboratories; Gaithersburg, MD). The plates are incubated at room temperature for 5-20 minutes, depending on the Fcγ receptors tested, to allow color development. The reaction is terminated with 1 M H3P04, and the absorbance at 450 nm was measured with a microplate reader (SpectraMax® 190, Molecular Devices; Sunnyvale, CA). Dose-response binding curves are generated by plotting the mean absorbance values from the duplicates of antibody dilutions against the concentrations of the antibody. The half maximal effective concentration (EC50) to the Fcγ receptor binding is determined after fitting the binding curve with a four-parameter equation using SoftMax Pro (Molecular Devices).

Compared to the control, antibodies that induce cell death were selected by the loss of cell membrane integrity measured by, for example, propidium iodide (PI), trypan blue or 7AAD uptake. A PI uptake assay could be performed in the absence of complement and immune effector cells. OX40 expressing cells were incubated with medium alone or medium containing about 10 μg/ml of appropriate monoclonal antibody. The cells were incubated for a period of time (e.g., 1 or 3 days). Following each treatment, cells were washed and aliquoted. In some embodiments, cells were aliquoted into 35 mm strainer-capped 12×75 tubes (1 ml per tube, 3 tubes per treatment group) for cell clumps removal. The PI solution was added to each tube at 10 μg/ml. Samples may be analyzed using a FACSCAN™ flow cytometer and FACSCONVERT™ CellQuest software (Becton Dickinson).

Cells for use in any of the above in vitro assays include cells or cell lines that naturally express OX40 or have been engineered to express OX40. Such cells include activated T cells, Treg cells, and activated memory T cells that naturally express OX40. Such cells also include cell lines that express OX40 and cell lines that do not normally express OX40 but have been transfected with polynucleotide encoding OX40. Exemplary cell lines provided herein for use in any of the above in vitro assays include transgenic BT474 cells (a human breast cancer cell line) that express human OX40.

It is understood that an immunoconjugate of the invention can be used to replace or supplement an anti-OX40 antibody for any of the above assays.

It is understood that anti-OX40 antibody and some other therapeutic agents can be used for any of the above assays.

### Formulations and Uses

The antibody, or antigen-binding fragment thereof, of the invention can be formulated as a pharmaceutical composition. The pharmaceutical composition may further comprise a pharmaceutically acceptable carrier, excipient, and/or stabilizer (Remington: The Science and practice of Pharmacy 20th Ed., 2000, Lippincott Williams and Wilkins, Ed. K. E. Hoover), in the form of lyophilized formulation or aqueous solution. Acceptable carriers, excipients, or stabilizers are nontoxic to recipients at the given dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight polypeptides (less than about 10 residues); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™, or polyethylene glycol (PEG). Pharmaceutically acceptable excipients are further described herein.

The antibody, or antigen-binding fragment thereof, of the invention can be used for various therapeutic or diagnostic purposes. For example, the antibody, or antigen-binding fragment thereof, of the invention may be used as an affinity purification agents (e.g., for in vitro purification) and a diagnostic agent (e.g., for detecting expression in specific cells, tissues, or serum).

Exemplary therapeutic uses of the antibody or antigen-binding fragment thereof, of the invention include treating a cancer. The antibody, or antigen-binding fragment thereof, of the invention may also be used for the prevention of a disease.

For therapeutic applications, the antibody or antigen-binding fragment thereof of the invention can be administered to a mammal, especially a human by conventional techniques, such as intravenously (as a bolus or by continuous infusion over a period of time), intramuscularly, intraperitoneally, intra-cerebrospinally, subcutaneously, intra-articularly, intrasynovially, intrathecally, orally, topically, or by inhalation. The antibody or antigen-binding fragment thereof of the invention can also be administered by intra-tumoral, peri-tumoral, intra-lesional, or peri-lesional routes.

In certain embodiments, the antibody or antigen-binding fragment thereof of the invention is administered subcutaneously. In certain embodiments, the antibody or antigen-binding fragment thereof of the invention is administered intravenously.

The pharmaceutical compositions may be administered to a subject in need thereof at a frequency that may vary with the severity of a disease. In the case of preventive therapy, the frequency may vary depending on the subject's susceptibility or predisposition to a disease.

The compositions may be administered to patients in need thereof as a bolus or by continuous infusion. For example, a bolus administration of an antibody presented as a Fab fragment may be in an amount of 0.0025 to 100 mg/kg of body weight, 0.025 to 0.25 mg/kg, 0.010 to 0.10 mg/kg or 0.10-0.50 mg/kg. For continuous infusion, an antibody presented as an Fab fragment may be administered at 0.001 to 100 mg/kg of body weight/minute, 0.0125 to 1.25 mg/kg/min, 0.010 to 0.75 mg/kg/min, 0.010 to 1.0 mg/kg/min. or 0.10-0.50 mg/kg/min for a period of 1-24 hours, 1-12 hours, 2-12 hours, 6-12 hours, 2-8 hours, or 1-2 hours.

For a full-length antibody (with full constant regions), the administration dose may range from about 1 mg/kg to about 10 mg/kg, about 2 mg/kg to about 10 mg/kg, about 3 mg/kg to about 10 mg/kg, about 4 mg/kg to about 10 mg/kg, about 5 mg/kg to about 10 mg/kg, about 1 mg/kg to about 20 mg/kg, about 2 mg/kg to about 20 mg/kg, about 3 mg/kg to about 20 mg/kg, about 4 mg/kg to about 20 mg/kg, about 5 mg/kg to about 20 mg/kg, about 1 mg/kg or more, about 2 mg/kg or more, about 3 mg/kg or more, about 4 mg/kg or more, about 5 mg/kg or more, about 6 mg/kg or more, about 7 mg/kg or more, about 8 mg/kg or more, about 9 mg/kg or more, about 10 mg/kg or more, about 11 mg/kg or more, about 12 mg/kg or more, about 13 mg/kg or more, about 14 mg/kg or more, about 15 mg/kg or more, about 16 mg/kg or more, about 17 mg/kg or more, about 19 mg/kg or more, or about 20 mg/kg or more. Depending upon the severity of the condition, the frequency of the administration could range from three times per week to once every two or three weeks.

Additionally, the compositions may be administered subcutaneously. For example, a dose of 1 to 100 mg anti-OX40 antibody can be administered to a subject subcutaneously or intravenously twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, or once every three months.

In certain embodiments, the half-life of the anti-OX40 antibody in human is about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, from about 5 days to about 40 days, from about 5 days to about 35 days, from about 5 days to about 30 days, from about 5 days to about 25 days, from about 10 days to about 40 days, from about 10 days to about 35 days, from about 10 days to about 30 days, from about 10 days to about 25 days, from about 15 days to about 40 days, from about 15 days to about 35 days, from about 15 days to about 30 days, or from about 15 days to about 25 days.

In certain embodiments, the pharmaceutical composition is administered subcutaneously or intravenously every 2-6 weeks, at a dose of about 0.1 mg/kg to about 10 mg/kg, about 0.5 mg/kg to about 10 mg/kg, about 1 mg/kg to about 10 mg/kg, about 1.5 mg/kg to about 10 mg/kg, about 2 mg/kg to about 10 mg/kg, about 0.1 mg/kg to about 8 mg/kg, about 0.5 mg/kg to about 8 mg/kg, about 1 mg/kg to about 8 mg/kg, about 1.5 mg/kg to about 8 mg/kg, about 2 mg/kg to about 8 mg/kg, about 0.1 mg/kg to about 5 mg/kg, about 0.5 mg/kg to about 5 mg/kg, about 1 mg/kg to about 5 mg/kg, about 1.5 mg/kg to about 5 mg/kg, about 2 mg/kg to about 5 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, or about 10.0 mg/kg.

In one embodiment, the pharmaceutical composition is administered subcutaneously or intravenously every 2-6 weeks, at a dose of about 2.0 mg/kg. In another embodiment, the pharmaceutical composition is administered subcutaneous or intravenously every 2-6 weeks, at a dose of about 2.0 mg/kg to about 10.0 mg/kg.

In one exemplary embodiment, pharmaceutical composition is administered subcutaneously every 2 weeks.

The antibody or antigen-binding fragment thereof of the invention can be used by itself or in combination with other therapies for the treatment of cancers.

### Definitions

Unless otherwise defined herein, scientific and technical terms used in connection with the present invention shall have the meanings that are commonly understood by those of ordinary skill in the art. Further, unless otherwise specified in the description, singular terms shall include pluralities and plural terms shall include the singular. Generally, the terminologies and technologies related to cell and tissue culture, molecular biology, immunology, microbiology, genetics, protein and nucleic acid chemistry, and hybridization, as described herein are well-known and commonly used by one of ordinary skill in the art.

The term “antigen-binding fragment” of an antibody (or simply “antibody portion” or “antibody fragment”), as used herein, refers to one or more fragments of a full-length antibody that retain the ability to specifically bind to an antigen (preferably with substantially the same binding affinity). Examples of an antigen-binding fragment includes (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR), disulfide-linked Fvs (dsFv), and anti-idiotypic (anti-Id) antibody and intrabody. Furthermore, although the two domains of the Fv fragment, VL and VH, are encoded by two different genes, they can be joined, using recombinant methods, by a synthetic linker that enables them to be made as a single protein chain in which the VL and VH regions pair to form monovalent molecules (known as single chain Fv (scFv)); see e.g., Bird et al. Science 242:423-426 (1988) and Huston et al., Proc. Natl. Acad. Sci. USA 85:5879-5883 (1988). Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. Proc. Natl. Acad. Sci. USA 90:6444-6448 (1993); Poljak et al., 1994, Structure 2: 1121-1123).

The term “variable domain” of an antibody, as used herein, refers to the variable region of the antibody light chain (VL) or the variable region of the antibody heavy chain (VH), either alone or in combination. As known in the art, the variable regions of the heavy and light chains each consist of three complementarity determining regions (CDRs) and are connected by four framework regions (FR), which contribute to the formation of the antigen-binding site of antibodies.

Residues in a variable domain are numbered according to Kabat Numbering, which is a numbering scheme used for heavy chain variable domains or light chain variable domains of an antibody. See, Kabat et al., Sequences of Proteins of Immunological Interest, 5th Ed. Public Health Service, National Institutes of Health, Bethesda, MD. (1991). Using this numbering system, the actual linear amino acid sequence may contain fewer or additional amino acids corresponding to a shortening of, or an insertion into, a FR or CDR of the variable domain. For a given antibody, the Kabat numbering of the residues may be determined by one or more homologous regions through the alignment of the antibody with the “standard” Kabat sequence. Various algorithms for assigning Kabat numbering are available. Unless stated otherwise, the algorithm used herein is the 2012 release of Abysis (abysis dot org).

The positions of certain specific amino acid residues of an antibody (such as paratope residues) are also determined by Kabat Numbering.

The term “Complementarity Determining Regions” (CDRs) can be identified by well-known Kabat, Chothia, the accumulation of Kabat and Chothia, AbM, the definitions of contact and/or conformational, or the definition of any method for CDR measurement. See, e.g., Kabat et al., 1991, Sequences of Proteins of Immunological Interest, 5th ed. (hypervariable regions); Chothia et al., 1989, Nature 342:877-883 (structural loop structures). The AbM definition of CDRs is a compromise between Kabat and Chothia and the uses of the Oxford Molecular's AbM antibody modeling software (Accelrys®). The “contact” definition of CDRs is based on the observed antigen contacts, setting forth in MacCallum et al., 1996, J. Mol. Biol., 262:732-745. The “conformational” definition of CDRs is based on residues that make enthalpic contributions to antigen binding (see, e.g., Makabe et al., 2008, Journal of Biological Chemistry, 283: 1 156-1 166). Still other CDR boundary definitions may not strictly follow one of the above approaches, but will nonetheless overlap with at least a portion of the Kabat CDR definition. These regions may be shortened or lengthened based on the prediction or experimental findings if the particular residues or groups of residues or even entire CDRs do not significantly impact antigen binding. As used herein, a CDR may be based on any definition methods known in the art, including a combination of different approaches.

The term “epitope” refers to the area or region of an antigen (Ag) to which an antibody specifically binds, e.g., an area or region comprising amino acid residues that interact with the antibody (Ab). Epitopes can be linear or non-linear (e.g., conformational).

An antibody or antigen-binding fragment thereof substantially binds to the same epitope of another antibody or antigen-binding fragment thereof, when the binding of the corresponding antibodies or antigen-binding fragments thereof are mutually exclusive. That is, binding of one antibody or antigen-binding fragment thereof excludes simultaneous or consecutive binding of the other antibodies or antigen-binding fragments thereof. Epitopes are said to be unique, or not substantially the same, if the antigen is able to simultaneously bind to both the corresponding antibodies, or antigen-binding fragments thereof.

The term “paratope” is derived from the above definition of “epitope”, and refers to an antibody area or region to which an antigen binds, e.g., an area or region comprising residues that interacts with the antigen. A paratope may be linear or conformational (such as discontinuous residues in CDRs).

The epitope/paratope for a given antibody/antigen binding pair can be defined and characterized at different levels of detail using a variety of experimental and computational epitope mapping methods. These methods include mutagenesis, X-ray crystallography, Nuclear Magnetic Resonance (NMR) spectroscopy, Hydrogen/deuterium exchange Mass Spectrometry (HX-MS) and various competitive binding methods. As each of these methods relies on the unique antibody-antigen binding, the description of an epitope is closely related to the method by which it has been determined. Thus, the epitope/paratope for a given antibody/antigen pair will be defined differently depending on the mapping method employed.

At its most detailed level, the epitope/paratope for the interaction between an antibody (Ab) and antigen (Ag) can be defined based on the spatial coordinates of atomic interaction during Ag-Ab binding, as well as information about their relative contributions to the binding thermodynamics. At one level, an epitope/paratope residue can be characterized by the spatial coordinates of atomic interaction between the Ag and Ab. In one aspect, the epitope/paratope residue can be defined by a specific criteria, e.g., the atom distance between the Ab and Ag (e.g., a distance of equal to or less than the distance between a heavy atom of the cognate antibody and a heavy atom of the antigen). In another aspect, an epitope/paratope residue can be characterized based on its participation in the interaction between a hydrogen bond and the cognate antibody/antigen or a water molecule that is also hydrogen bonded to the cognate antibody/antigen (water-mediated hydrogen bonding). In another aspect, an epitope/paratope residue can be characterized as forming a salt bridge with a residue of the cognate antibody/antigen. In yet another aspect, an epitope/paratope residue can be characterized as a residue having a non-zero change in buried surface area (BSA) due to interaction with the cognate antibody/antigen. At a less detailed level, an epitope/paratope can be characterized based on its function, e.g., by competition binding with other Abs. The epitope/paratope can also be defined more generically as comprising amino acid residues for which substitution by another amino acid will alter the characteristics of the interaction between the Ab and Ag (e.g. alanine scanning).

From the fact that the descriptions and definitions of epitopes dependent on the method used for epitope mapping and the information at different levels of details, it follows that the comparison of epitopes for different Abs on the same Ag can similarly be conducted at different levels of details. For example, on the amino acid level, epitopes with the same set of amino acid residues, determined from an X-ray structure, are said to be identical.

Epitopes characterized by competitive binding are said to be overlapping if the binding of the corresponding antibodies are mutually exclusive, i.e., binding of one antibody excludes simultaneous or consecutive binding of another antibody; and epitopes are said to be separate (unique) if the antigen can simultaneously bind to both corresponding antibodies.

The epitope and paratope for a given antibody/antigen pair may be identified through some routine methods. For example, the general location of an epitope may be determined by assessing the ability of an antibody binding to the different fragments or variant polypeptides as more fully described previously elsewhere herein. The specific residues within OX40 that make contact with other specific residues within an antibody may also be determined using the routine methods. For example, antibody/antigen complex may be crystallized. The crystal structure may be determined and used to identify specific interaction sites between the antibody and antigen.

The terms “specifically binds” and “specific binding” are well-understood in the art, so are the methods to determine such specific binding. A molecule is said to exhibit “specific binding” if it reacts or associates with a particular cell or substance more frequently, more rapidly, with greater duration and/or with greater affinity than it does with alternative cells or substances. An antibody or antigen-binding fragment thereof “specifically binds” to a target if it binds with greater affinity, avidity, more readily, and/or with greater duration than it does with other substances.

For example, an antibody or antigen-binding fragment thereof that specifically binds to OX40 is one that binds to its cognate antigen with greater affinity, avidity, more readily, and/or with greater duration than it binds to other antigens. More specifically, an anti-OX40 antibody can specifically binds to the human OX40, but does not substantially recognize or bind to other molecules in a sample under a standard binding assay condition. It is also understood that an antibody or antigen-binding fragment thereof, which specifically binds a first target may or may not specifically bind to a second target. As such, the “specific binding” does not necessarily require (although it can include) exclusive binding. Generally, but not necessarily, reference to “binding” means specific binding.

A variety of methods may be used to select an antibody, or antigen-binding fragment thereof, that specifically binds a molecule of interest. For example, solid-phase ELISA immunoassay, immunoprecipitation, Biacore™ (GE Healthcare), KinExA, fluorescence-activated cell sorting (FACS), Octet™ (ForteBio, Inc.) and Western blot analysis are among many methods that may be used to identify an antibody, or antigen-binding fragment thereof, that specifically binds to an antigen. Typically, a specific binding will generate a signal that is at least twice of the background signal or noise, more often, at least 10 times of the background, at least 50 times of the background, at least 100 times of the background, at least 500 times of the background, at least 1000 of times of the background, or at least 10,000 times of the background.

The specificity of an antibody binding may be assessed by comparing the K_{D} values of a specific binding between an antibody and the OX40 with the K_{D} value of a OX40-control binding, wherein the control is known not to bind to OX40. In general, an antibody is said to “specifically bind” to an antigen when the K_{D} is about ×10⁻⁵ M or less.

An antibody or antigen-binding fragment thereof “does not substantially bind” to an antigen when it does not bind to said antigen with greater affinity, avidity, more readily, and/or with greater duration than it binds to other antigens. Typically, the binding will generate signal that is no greater than twice of the background signal or noise. In general, it binds to the antigen with a K_{D} of 1×10⁻⁴ M or more, 1×10⁻³ M or more, 1×10⁻² M or more, or 1×10⁻¹ M or more.

The term “compete”, as used herein with regard to an antibody, means that binding of a first antibody, or an antigen-binding portion thereof, to an antigen reduces the subsequent binding of the same antigen to a second antibody or antigen-binding portion thereof. In general, the binding of a first antibody creates steric hindrance, conformational change, or reduced the binding of a second antibody to a common epitope (or portion thereof). Standard competitive binding assays may be used to determine whether two antibodies compete with each other.

One appropriate assay for antibody competition analysis involves the use of the Biacore technology, which can measure the extent of interactions using surface plasmon resonance (SPR) technology, typically using a biosensor system (such as a BIACORE® system). For example, SPR can be used in an in vitro competitive binding inhibition assay to determine the inhibitive ability of one antibody to the binding of a second antibody. Another antibody competition measurement is based on ELISA method. Furthermore, a high throughput method for “binning” antibodies based upon antibody competition is described in WO2003/48731. Competition is present if one antibody, or antigen-binding fragment thereof, reduces the binding of another antibody, or antigen-binding fragment thereof, to OX40. For example, a sequential binding competition assay may be used by adding different antibodies sequentially. Specifically, the first antibody may be added to a level close to binding saturation. Then, the second antibody is added. If the binding of second antibody to OX40 is not detectable, or is significantly reduced (e.g., at least about 10%, at least about 20%, at least about 30%, at least about 40%, at least about 50%, at least about 60%, at least about 70%, at least about 80%, or at least about 90% reduction) as compared to a parallel assay in the absence of the first antibody (which value can be set as 100%), the two antibodies are considered as competing with each other.

A competitive binding assay can also be conducted in which the binding of an antibody to an antigen is compared to the binding of the target by another binding partner of that target, such as another antibody or a soluble receptor that otherwise binds to the target. The concentration at which 50% inhibition occurs is known as the Ki. Under ideal conditions, the Kᵢ is equivalent to the K_{D}. Thus, in general, the measurement of Ki can conveniently be substituted to provide the upper limit of K_{D}. Binding affinities associated with different molecular interactions, e.g., comparison of the binding affinity of different antibodies for a given antigen, may be evaluated by the comparison of the K_{D} values for the individual antibody/antigen complexes. The K_{D} values for antibodies or other binding partners can be determined using methods well established in the art.

The term “Fc fusion” protein is a protein wherein one or more polypeptides are operably linked to an Fc polypeptide. An Fc fusion combines the Fc region of an immunoglobulin with a fusion partner. The “Fc region” may be a native sequence of the Fc region or a variant of the Fc region. Although, the boundaries of the Fc region of an immunoglobulin heavy chain might vary, the human IgG heavy chain Fc region is usually defined to stretch from an amino acid residue at position Cys226 or Pro230 to the carboxyl-terminus thereof. The numbering of the residues in the Fc region is that of the EU index as described in Kabat et al., Sequences of Proteins of Immunological Interest, 5th Ed. Public Health Service, National Institutes of Health, Bethesda, Md., 1991. The Fc region of an immunoglobulin generally comprises two constant domains, CH₂ and CH₃. As is known in the art, an Fc region can exist in either dimer or monomeric form.

The term “therapeutically effective amount” means an amount of an anti-OX40 antibody or antigen-binding fragment thereof, or a combination comprising such antibody or antigen-binding fragment thereof, that is of sufficient quantity to achieve the intended purpose. The precise amount will depend upon numerous factors, including, but not limited to, the components and physical characteristics of the therapeutic composition, intended patient population, individual patient specifications, and the like, and can be determined by one skilled in the art.

The term “treatment” includes preventive and/or therapeutic treatments. If it is administered prior to the appearance of any clinical signs or symptoms of a disease, disorder, or condition, the treatment is considered as preventive. Therapeutic treatment includes, e.g., ameliorating or reducing the severity of a disease, disorder, or condition, or shortening the length of the disease, disorder, or condition.

The term “about”, as used herein, refers to +/−10% of a value.

### Therapeutic Methods and Compositions

Any of the anti-human OX40 antibodies provided herein may be used in a given therapeutic method.

In one aspect, an anti-human OX40 agonist antibody provided herein is used for a pharmaceutical composition. In another aspect, an anti-human OX40 agonist antibody provided herein is used for the treatment of cancers. In some embodiments, an anti-human OX40 agonist antibody provided herein is used as a treatment method. In other embodiments, the invention provides a method for the treatment a subject with cancer comprising administering to the subject a therapeutically effective amount of the anti-human agonist OX40 antibody. In one such embodiment, the method further comprises administering to the subject a therapeutically effective amount of at least one additional therapeutic agent, e.g., as described below.

In one aspect, provided is an anti-human OX40 agonist antibody for the use of enhancing the immune function (e.g., by upregulating cell-mediated immune responses) of an individual having cancer comprising administering to the individual a therapeutically effective amount of the anti-human OX40 agonist antibody. In another aspect, provided is an anti-human OX40 agonist antibody for the use of enhancing T cell function in an individual having cancer comprising administering to the individual a therapeutically effective amount of the anti-human OX40 agonist antibody. In another aspect, provided is an anti-human OX40 agonist antibody for the use of depleting the human OX40-expressing cells (e.g., OX40 expressing T cells, e.g., OX40 expressing Treg) comprising administering to the individual a therapeutically effective amount of the anti-human OX40 agonist antibody. In some embodiments, the depletion is accomplished through ADCC. In other embodiments, the depletion is accomplished through phagocytosis. Provided is an anti-human OX40 agonist antibody for the treatment of an individual having tumor immunity.

In further aspects, provided is an anti-human OX40 agonist antibody for the treatment of infection (e.g., bacteria or virus or other pathogen induced infections). In some embodiments, the invention provides a method for the treatment of an individual with an infection comprising administering to the individual a therapeutically effective amount of the anti-human agonist OX40 antibody. In one embodiment, the infections are induced by virus and/or a bacteria. In another embodiment, the infections are induced by other pathogens.

In another embodiment, the invention provides the use of a pharmaceutical composition that comprises the manufacture or preparation of an anti-OX40 antibody. In one embodiment, the pharmaceutical composition is for the treatment of cancers. In another embodiment, the invention provides a method for the treatment of an individual with cancers comprising administering to the individual a therapeutically effective amount of the pharmaceutical composition. In one such embodiment, the method further comprises administering to the individual an effective amount of at least one additional therapeutic agent, e.g., as described below.

In one aspect, the pharmaceutical composition is used to enhance the immune function (e.g., by upregulating cell-mediated immune responses) of an individual having cancer comprising administering to the individual a therapeutically effective amount of the pharmaceutical composition. In another aspect, the pharmaceutical composition is for the use of enhancing T cell function in an individual having cancer comprising administering to the individual a therapeutically effective amount of the pharmaceutical composition. In some embodiments, the T cell dysfunctional disorder is a cancer. In one aspect, the pharmaceutical composition is for the use of depleting the human OX40-expressing cells (e.g., cell expressing high level of OX40, e.g., OX40 expressing T cells) comprising administering to the individual a therapeutically effective amount of the pharmaceutical composition. In one embodiment, the depletion is accomplished through ADCC. In another embodiment, the depletion is accomplished through phagocytosis. In another aspect, the pharmaceutical composition is for the treatment of an individual with tumor immunity.

In further aspects, provided is a pharmaceutical composition used for the treatment of infections (e.g., infections induced by bacteria, virus, or other pathogens). In certain embodiments, the pharmaceutical composition is used for treating an individual having an infection comprising administering to the individual a therapeutically effective amount of the pharmaceutical composition. In one embodiment, the infection is induced by virus and/or bacteria. In another embodiment, the infection is induced by some other pathogens.

In a further aspect, the invention provides a method for treating cancer. In one embodiment, the method comprises administering to an individual with cancer a therapeutically effective amount of an anti-OX40 antibody. In one such embodiment, the method further comprises administering to the individual an effective amount of at least one additional therapeutic agent, as described below. An “individual” according to any of the above embodiments may be a human.

In one aspect, provided is a method for enhancing immune function (e.g., by upregulating cell-mediated immune responses) in an individual with cancer comprising administering to the individual a therapeutically effective amount of the anti-human agonist OX40 antibody. In one aspect, provided is a method for enhancing T cell function in an individual having cancer comprising administering to the individual a therapeutically effective amount of the anti-human agonist OX40 antibody. In another aspect, provided is a method for depleting human OX40-expressing cells (e.g., cells that express high level of OX40, e.g., OX40 expressing T cells) comprising administering to the individual a therapeutically effective amount of the anti-human agonist OX40 antibody. In one embodiment, the depletion is accomplished through ADCC. In another embodiment, the depletion is accomplished through phagocytosis. In another embodiment, provided is an anti-human OX40 agonist antibody for the treatment of an individual with tumor immunity.

In some embodiments, cancers described herein further include, but not limited to, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone—MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, and angiocentric lymphoma.

In other embodiments, examples of the cancer further include, but not limited to, B-cell proliferative disorders, which further include, but not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In some embodiments of any methods described herein, the cancer is a B-cell proliferative disorder. In other embodiments, the B-cell proliferative disorder is a lymphoma, non-Hodgkins lymphoma (NHL), aggressive NHL, relapsed aggressive NHL, relapsed indolent NHL, refractory NHL, refractory indolent NHL, chronic lymphocytic leukemia (CLL), small lymphocytic lymphoma, leukemia, hairy cell leukemia (HCL), acute lymphocytic leukemia (ALL), or mantle cell lymphoma. In some embodiments, the B-cell proliferative disorder is a NHL, such as indolent NHL and/or aggressive NHL. In another embodiment, the B-cell proliferative disorder is an indolent follicular lymphoma or a diffuse large B-cell lymphoma.

In a further aspect, the invention provides some pharmaceutical formulations comprising any anti-OX40 antibodies described in any of the above therapeutic methods. In one embodiment, a pharmaceutical formulation comprises any of the anti-OX40 antibodies provided herein and a pharmaceutically acceptable carrier. In another embodiment, a pharmaceutical formulation comprises any of the anti-OX40 antibodies provided herein and at least one additional therapeutic agent, e.g., as described below.

In some embodiments of any methods described herein, the anti-human OX40 agonist antibodies inhibit tumor immunity by inhibiting Treg function (e.g., inhibiting the suppressive function of Tregs), killing OX40 expressing cells (e.g., cells expressing high levels of OX40), and increasing effector T cell function and/or increasing memory T cell function. In some embodiments of any methods described herein, the anti-human OX40 agonist antibodies treat cancer by inhibiting Treg function (e.g., inhibiting the suppressive function of Tregs), killing OX40 expressing cells (e.g., cells expressing high levels of OX40), and increasing effector T cell function and/or increasing memory T cell function. In some embodiments of any methods described herein, the anti-human OX40 agonist antibodies enhance immune function by inhibiting Treg function (e.g., inhibiting the suppressive function of Tregs), killing OX40 expressing cells (e.g., cells expressing high levels of OX40), and increasing effector T cell function, and/or increasing memory T cell function. In some embodiments of any methods described herein, the anti-human OX40 agonist antibodies enhance T cell function by inhibiting Treg function (e.g., inhibiting the suppressive function of Tregs), killing OX40 expressing cells (e.g., cells expressing high levels of OX40), increasing effector T cell function, and/or increasing memory T cell function.

In some embodiments of any methods described herein, the anti-human OX40 agonist antibody is a inhibitory anti-human agonist antibody. In some embodiments, treatment with the anti-human OX40 agonist antibody results in cell depletion (e.g., depletion of OX40-expressing cells or cells expressing high levels of OX40). In one embodiment, depletion is accomplished through ADCC. In another embodiment, depletion is accomplished through phagocytosis.

In some embodiments of any methods described herein, administration of the anti-human OX40 agonist antibody inhibits Treg function by inhibiting Treg suppression of effector and/or memory T cell function (in some embodiments, effector T cell and/or memory T cell proliferation and/or cytokine secretion). In some embodiments of any methods described herein, administration of the anti-human OX40 agonist antibody increases effector T cell proliferation. In some embodiments of any methods described herein, administration of the anti-human OX40 agonist antibody increases memory T cell proliferation. In some embodiments of any methods described herein, administration of the anti-human OX40 agonist antibody increases effector T cell cytokine production (e.g., interferon γ-production). In some embodiments of any methods described herein, administration of the anti-human OX40 agonist antibody increases memory T cell cytokine production (e.g., interferon γ-production). In some embodiments of any methods described herein, administration of the anti-human OX40 agonist antibody increases CD⁴⁺ effector T cell proliferation and/or CD8⁺ effector T cell proliferation. In some embodiments of any methods described herein, administration of the anti-human OX40 agonist antibody increases memory T cell proliferation (e.g., CD4⁺ memory T cell proliferation). In some embodiments, the administration of the anti-human OX40 agonist antibody enhances the proliferation, cytokine secretion and/or cytolytic activity of the CD4⁺ effector T cells in the individual.

In some embodiments of any methods described herein, the number of CD4⁺ effector T cells is elevated compared to the number prior to administration of the anti-human OX40 agonist antibody. In some embodiments, CD4⁺ effector T cell cytokine secretion is elevated compared to the level prior to administration of the anti-human OX40 agonist antibody. In some embodiments of any methods described herein, the administration of the anti-human OX40 agonist antibody enhances the proliferation, cytokine secretion, and/or cytolytic activity of the CD8⁺ effector T cells in the individual. In some embodiments, the number of CD8⁺ effector T cells is elevated compared to the number prior to administration of the anti-human OX40 agonist antibody. In some embodiments, CD8⁺ effector T cell cytokine secretion is elevated compared to the level prior to administration of the anti-human OX40 agonist antibody.

In some embodiments of any methods described herein, the anti-human OX40 agonist antibody binds to human effector cells, e.g., binds to FcγR expressed in human effector cells. In one embodiment, the human effector cell performs ADCC effector function. In another embodiment, the human effector cell performs phagocytosis effector function.

In some embodiments of any methods described herein, the anti-human OX40 agonist antibody comprising an IgG1Fc polypeptide variant, e.g., a mutation that eliminates its binding to the human effector cells (e.g., a DANA or N297G mutation) has diminished activity (e.g., CD4⁺ effector T cell function, e.g., proliferation), compared to the anti-human OX40 agonist antibody with the native sequence of the IgG1Fc region. In some embodiment, the anti-human OX40 agonist antibody comprising an IgG1 Fc polypeptide variant, e.g., a mutation that eliminates its binding to the human effector cells (e.g., a DANA or N297G mutation), does not possess substantial activity (e.g., CD4⁺ effector T cell function, e.g., proliferation).

In some embodiments of any methods described herein, antibody cross-linking is required for anti-human OX40 agonist antibody to function. In some embodiments, the function is to stimulate CD4⁺ effector T cell proliferation. In some embodiments, antibody cross-linking ability is determined by measuring the amount of anti-human OX40 agonist antibody adhered to a solid surface (e.g., a cell culture plate). In some embodiments, antibody cross-linking ability is determined by introducing a mutation in the antibody's IgG1 Fc region (e.g., a DANA or N297S mutation) and testing the function of the mutant antibody.

In some embodiments of any methods described herein, the administration of the anti-human OX40 agonist antibody enhanced the proliferation and/or cytokine secretion of the memory T cells in an individual. In some embodiments, the administration of the anti-human OX40 agonist antibody increased the number of memory T cells. In some embodiments, the administration of the anti-human OX40 agonist antibody increased the cytokine secretion (level) of memory T cell. In some embodiments of any methods described herein, the administration of the anti-human OX40 agonist antibody have decreased Treg suppression of effector T cell (e.g., proliferation and/or cytokine secretion) in an individual. In some embodiments, the number of effector T cells is elevated compared to the number prior to the administration of the anti-human OX40 agonist antibody. In some embodiments, effector T cell cytokine secretion (level) is elevated compared to the level prior to the administration of the anti-human OX40 agonist antibody.

In some embodiments of any methods described herein, the number of intratumoral (infiltrating) CD4⁺ effector T cells (e.g., total number of CD4⁺ effector T cells, or e.g., the percentage of CD4⁺ cells in CD45⁺ cells) has increased compared to the number prior to the administration of the anti-human OX40 agonist antibody. In some embodiments of any methods described herein, the number of intratumoral (infiltrating) CD4⁺ effector T cells that express interferon γ—(e.g., total interferon γ-expressing CD4⁺ cells, or e.g., the percentage of interferon γ-expressing CD4⁺ cells in total CD4⁺ cells) has increased compared to their level prior to the administration of the anti-human OX40 agonist antibody.

In some embodiments of any methods described herein, the number of intratumoral (infiltrating) CD8⁺ effector T cells (e.g., total number of CD8⁺ effector T cells, or e.g., percentage of CD8⁺ in CD45⁺ cells) has increased compared to the number prior to the administration of the anti-human OX40 agonist antibody. In some embodiments of any methods described herein, the number of intratumoral (infiltrating) CD8⁺ effector T cells that express interferon γ—(e.g., the percentage of CD8⁺ cells that express interferon γ—in total CD8⁺ cells) has increased compared to the number prior to the administration of anti-human OX40 agonist antibody.

In some embodiments of any methods described herein, the number of intratumoral (infiltrating) Treg (e.g., total number of Treg or e.g., the percentage of Fox3p+ cells in CD4⁺ cells) has decreased compared to the number prior to administration of anti-human OX40 agonist antibody.

In some embodiments of any methods described herein, the administration of anti-human OX40 agonist antibody is in combination with the administration of a tumor antigen. In some embodiments, the tumor antigen comprises protein. In some embodiments, the tumor antigen comprises nucleic acid. In some embodiments, the tumor antigen is a tumor cell.

In some embodiments of any methods described herein, the cancer possesses human effector cells (e.g., infiltrated by human effector cells). Methods for detecting human effector cells are well known in the art, including, e.g., IHC. In some embodiments, the cancer is featured by high levels of human effector cells. In some embodiments, the human effector cells are one or more of the NK cells, macrophages, and monocytes. In some embodiments, the cancer is any cancer described herein. In some embodiments, the cancer is non-small cell lung cancer (NSCLC), glioblastoma, neuroblastoma, melanoma, breast carcinoma (e.g. triple-negative breast cancer), gastric cancer, colorectal cancer (CRC), or hepatocellular carcinoma.

In some embodiments of any methods described herein, the cancer possesses cells expressing FcR (e.g., is infiltrated by cells expressing FcR). Methods for FcR detection are well known in the art, including, e.g., IHC. In some embodiments, the cancer possesses high levels of FcR expressing cells. In some embodiments, the FcR is FcγR. In some embodiments, the FcR is active FcγR. In some embodiments, the cancer is non-small cell lung cancer (NSCLC), glioblastoma, neuroblastoma, melanoma, breast carcinoma (e.g. triple-negative breast cancer), gastric cancer, colorectal cancer (CRC), or hepatocellular carcinoma.

An “individual” or a “subject” according to any of the above embodiments is preferably a human.

Antibodies of the invention can be used either alone or in combination with other therapeutic agents. For instance, an antibody described herein may be co-administered with at least one additional therapeutic agent.

Such combination therapies described above include both combined administration (where two or more therapeutic agents are included in the same or separate formulations) and separate administration, wherein the administration of the antibody described herein can occur prior to, simultaneously, and/or following the administration of the additional therapeutic agent or agents. In one embodiment, the administration of the anti-OX40 antibody and the administration of an additional therapeutic agent occur within about one month, or within about one, two, or three weeks, or within about one, two, three, four, five, or six days, of each other. Antibodies of the invention can also be used in combination with a radiation therapy.

In some embodiments, an anti-human OX40 agonist antibody may be administered in combination with a chemotherapy or a chemotherapeutic agent. In some embodiments, an anti-human OX40 agonist antibody may be administered in combination with a radiation therapy or a radiotherapeutic agent. In some embodiments, an anti-human OX40 agonist antibody may be administered in combination with a targeted therapy or a targeted therapeutic agent. In some embodiments, an anti-human OX40 agonist antibody may be administered in combination with an immunotherapy or an immunotherapeutic agent, for example, a monoclonal antibody.

In some embodiments, an anti-human OX40 agonist antibody may be administered in combination with a PARP inhibitor (e.g., Olaparanib, Rucaparib, Niraparib, Cediranib, BMN673, Veliparib), Trabectedin, nab-paclitaxel (albumen-bound paclitaxel, ABRAXANE), Trebananib, Pazopanib, Cediranib, Palbociclib, everolimus, fluoropyrimidine (e.g., FOLFOX, FOLFIRI), IFL, regorafenib, Reolysin, Alimta, Zykadia, Sutent, Torisel (temsirolimus), Inlyta (axitinib, Pfizer), Afinitor (everolimus, Novartis), Nexavar (sorafenib, Onyx/Bayer), Votrient, Pazopanib, axitinib, IMA-901, AGS-003, cabozantinib, Vinflunine, Hsp90 inhibitor (e.g., apatorsin), Ad-GM-CSF (CT-0070), Temozolomide, IL-2, IFNa, vinblastine, Thalomid, dacarbazine, cyclophosphamide, lenalidomide, azacytidine, lenalidomide, bortezomib (VELCADE), amrubicin, carfilzomib, pralatrexate, and/or enzastaurin.

In some embodiments, an anti-human OX40 agonist antibody may be administered in combination with a PD-1 axis binding antagonist, which includes, but not limited to, a PD-1 binding antagonist, a PD-L1 binding antagonist, and a PD-L2 binding antagonist. Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2. In some embodiments, the PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, the PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. In some embodiments, the PD-1 binding antagonist is an anti-PD-1 antibody (e.g., a human antibody, a humanized antibody, or a chimeric antibody). In some embodiments, the anti-PD-1 antibody is selected from MDX-1106 (nivolumab, OPDIVO), Merck 3475 (MK-3475, pembrolizumab), and KEYTRUDAWPCT-011 (Pidilizumab). In some embodiments, the PD-1 binding antagonist is an immunoadhesin (e.g., an immunoadhesin comprising an extracellular or PD-1 binding portion of PD-L1, or PD-L2 fused to a constant region (e.g., an Fc region of an immunoglobulin sequence)). In some embodiments, the PD-1 binding antagonist is AMP-224. In some embodiments, the PD-L1 binding antagonist is an anti-PD-L1 antibody. In some embodiments, the anti-PD-L1 binding antagonist is selected from YW243.55.S70, MPDL3280A, MEDI4736, and MDX-1105. MDX-1105, also known as BMS-936559, is an anti-PD-L1 antibody described in WO2007/005874. Antibody YW243.55.S70 is an anti-PD-L1 antibody described in WO2010/077634 A1. MDX-1106, also known as MDX-1106-04, ONO-4538, BMS-936558, or nivolumab, is an anti-PD-1 antibody described in WO2006/121168. Merck 3475, also known as MK-3475, SCH-900475, or pembrolizumab, is an anti-PD-1 antibody described in WO2009/114335. CT-011, also known as hBAT, hBAT-1, or pidilizumab, is an anti-PD-1 antibody described in WO2009/101611. AMP-224, also known as B7-DCIg, is a PD-L2—Fc fusion soluble receptor described in WO2010/027827 and WO2011/066342. In some embodiments, the anti-PD-1 antibody is MDX-1106. Alternative names for “MDX-1106” include MDX-1 106-04, ONO-4538, BMS-936558, and nivolumab. In some embodiments, the anti-PD-1 antibody is nivolumab (CAS Registry Number: 946414-94-4).

In some embodiments, an anti-human OX40 agonist antibody may be administered in combination with an agonist targeting an activating co-stimulatory molecule. In some embodiments, an activating co-stimulatory molecule may include CD40, CD226, CD28, GITR, CD137, CD27, HVEM, or CD127. In some embodiments, the agonist targeted by an activating co-stimulatory molecule is an agonist antibody that binds to CD40, CD226, CD28, OX40, GITR, CD137, CD27, HVEM, or CD127. In some embodiments, an anti-human OX40 agonist antibody may be administered in combination with an antagonist targeting an inhibitory co-stimulatory molecule. In some embodiments, an inhibitory co-stimulatory molecule may include CTLA-4 (also known as CD152), PD-1, TIM-3, BTLA, VISTA, LAG-3, B7-H3, B7-H4, IDO, TIGIT, MICA/B, or arginase. In some embodiments, the antagonist targeted by an inhibitory co-stimulatory molecule is an antagonist antibody that binds to CTLA-4, PD-1, TIM-3, BTLA, VISTA, LAG-3 (e.g., LAG-3-IgG fusion protein (IMP321)), B7-H3, B7-H4, IDO, TIGIT, MICA/B, or arginase.

In some embodiments, an anti-human OX40 agonist antibody may be administered in combination with an antagonist targeting CTLA-4 (also known as CD152), e.g., a blocking antibody. In some embodiments, an anti-human OX40 agonist antibody may be administered in combination with ipilimumab (also known as MDX-010, MDX-101, or Yervoy®). In some embodiments, an anti-human OX40 agonist antibody may be administered in combination with tremelimumab (also known as ticilimumab or CP-675,206). In some embodiments, an anti-human OX40 agonist antibody may be administered in combination with an antagonist targeting B7-H3 (also known as CD276), e.g., a blocking antibody. In some embodiments, an anti-human OX40 agonist antibody may be administered in combination with MGA271. In some embodiments, an anti-human OX40 agonist antibody may be administered in combination with an antagonist targeting TGFP, e.g., metelimumab (also known as CAT-192), fresolimumab (also known as GC1008), or LY2157299.

In some embodiments, an anti-human OX40 agonist antibody may be administered in combination with a treatment comprising adoptive transfer of a T cell (e.g., a cytotoxic T cell or CTL) expressing a chimeric antigen receptor (CAR). In some embodiments, an anti-human OX40 agonist antibody may be administered in combination with UCART19. In some embodiments, an anti-human OX40 agonist antibody may be administered in combination with WT128z. In some embodiments, an anti-human OX40 agonist antibody may be administered in combination with KTE-C19 (Kite). In some embodiments, an anti-human OX40 agonist antibody may be administered in combination with CTL019 (Novartis). In some embodiments, an anti-human OX40 agonist antibody may be administered in conjunction with a treatment comprising adoptive transfer of a T cell comprising a dominant-negative TGF beta receptor, e.g, a dominant-negative TGF beta type II receptor. In some embodiments, an anti-human OX40 agonist antibody may be administered in combination with a treatment comprising a HERCREEM trial (see, e.g., ClinicalTrials.gov Identifier NCT00889954).

In some embodiments, an anti-human OX40 agonist antibody may be administered in combination with an antagonist targeting CD19. In some embodiments, an anti-human OX40 agonist antibody may be administered in combination with MOR00208. In some embodiments, an anti-human OX40 agonist antibody may be administered in combination with an antagonist directed against CD38. In some embodiments, an anti-human OX40 agonist antibody may be administered in combination with daratumumab.

In some embodiments, an anti-human OX40 agonist antibody may be administered in combination with an agonist targeting CD137 (also known as TNFRSF9, 4-1BB, or ILA), e.g., an activating antibody. In some embodiments, an anti-human OX40 agonist antibody may be administered in combination with urelumab (also known as BMS-663513). In some embodiments, an anti-human OX40 agonist antibody may be administered in combination with an agonist targeting CD40, e.g., an activating antibody. In some embodiments, an anti-human OX40 agonist antibody may be administered in combination with CP-870893. In some embodiments, an anti-human OX40 agonist antibody may be administered in combination with an agonist targeting OX40 (also known as CD134), e.g., an activating antibody. In some embodiments, an anti-human OX40 agonist antibody may be administered in combination with a different anti-OX40 antibody (e.g., AgonOX). In some embodiments, an anti-human OX40 agonist antibody may be administered in combination with an agonist targeting CD27, e.g., an activating antibody. In some embodiments, an anti-human OX40 agonist antibody may be administered in combination with CDX-1127. In some embodiments, an anti-human OX40 agonist antibody may be administered in combination with an antagonist targeting indoleamine-2,3-dioxygenase (IDO). In some embodiments, the IDO antagonist is 1-methyl-D-tryptophan (also known as 1-D-MT).

In some embodiments, an anti-human OX40 agonist antibody may be administered in combination with an agonist targeting CD137 (also known as TNFRSF9, 4-1BB, or ILA), e.g., an activating antibody. In some embodiments, an anti-human OX40 agonist antibody may be administered in combination with urelumab (also known as BMS-663513). In some embodiments, an anti-human OX40 agonist antibody may be administered in combination with an agonist targeting CD40, e.g., an activating antibody. In some embodiments, an anti-human OX40 agonist antibody may be administered in combination with CP-870893 or R07009789. In some embodiments, an anti-human OX40 agonist antibody may be administered in combination with an agonist combination OX40 (also known as CD134), e.g., an activating antibody. In some embodiments, an anti-human OX40 agonist antibody may be administered in combination with an agonist targeting CD27, e.g., an activating antibody. In some embodiments, an anti-human OX40 agonist antibody may be administered in combination with CDX-1127 (also known as varlilumab). In some embodiments, an anti-human OX40 agonist antibody may be administered in combination with an antagonist targeting indoleamine-2,3-dioxygenase (IDO). In one embodiment, the IDO antagonist is 1-methyl-D-tryptophan (also known as 1-D-MT). In another embodiment, the IDO antagonist is the one described in WO2010/005958 (the contents of which are expressly incorporated by record herein). In another embodiment, the IDO antagonist is 4-({2-[(Aminosulfonyl)amino]ethyl}amino)-N-(3-bromo-4-fluorophenyl)-N′-hydroxy-1,2,5-oxadiazole-3-carboximidamide (e.g., as described herein in Example 23 of WO2010/005958). In some embodiments the IDO antagonist is

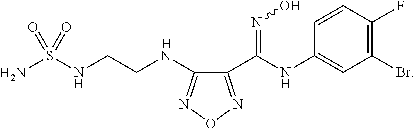

In some embodiments, the IDO antagonist is INCB24360. In some embodiments, the IDO antagonist is Indoximod (the D isomer of 1-methyl-tryptophan). In some embodiments, an anti-human OX40 agonist antibody may be administered in combination with an antibody-drug conjugate. In some embodiments, the antibody-drug conjugate comprises mertansine or monomethyl auristatin E (MMAE). In some embodiments, an anti-human OX40 agonist antibody may be administered in combination with an anti-NaPi2b antibody-MMAE conjugate (also known as DNIB0600A, RG7599 or lifastuzumab vedotin). In some embodiments, an anti-human OX40 agonist antibody may be administered in combination with trastuzumab emtansine (also known as T-DM1, ado-trastuzumab emtansine, or KADCYLA®, Genentech). In some embodiments, an anti-human OX40 agonist antibody may be administered in combination with an anti-MUC16 antibody-MMAE conjugate, DMUC5754A. In some embodiments, an anti-human OX40 agonist antibody may be administered in conjunction with an anti-MUC16 antibody-MMAE conjugate, DMUC4064A. In some embodiments, an anti-human OX40 agonist antibody may be administered in combination with an antibody-drug conjugate targeting the endothelin B receptor (EDNBR), e.g., an antibody targeting EDNBR conjugated with MMAE. In some embodiments, an anti-human OX40 agonist antibody may be administered in combination with an antibody-drug conjugate targeting the lymphocyte antigen 6 complex, locus E (Ly6E), e.g., an antibody targeting Ly6E conjugated with MMAE, (also known as DLYE5953A). In some embodiments, an anti-human OX40 agonist antibody may be administered in combination with polatuzumab vedotin. In some embodiments, an anti-human OX40 agonist antibody may be administered in combination with an antibody-drug conjugate targeting CD30. In some embodiments, an anti-human OX40 agonist antibody may be administered in combination with ADCETRIS (also known as brentuximab vedotin). In some embodiments, an anti-human OX40 agonist antibody may be administered in combination with polatuzumab vedotin.

In some embodiments, an anti-human OX40 agonist antibody may be administered in combination with an angiogenesis inhibitor. In some embodiments, an anti-human OX40 agonist antibody may be administered in combination with an antibody targeting VEGF, e.g., VEGF-A. In some embodiments, an anti-human OX40 agonist antibody may be administered in combination with bevacizumab (also known as AVASTIN®, Genentech). In some embodiments, an anti-human OX40 agonist antibody may be administered in combination with an antibody targeting angiopoietin 2 (also known as Ang2). In some embodiments, an anti-human OX40 agonist antibody may be administered in combination with MEDI3617. In some embodiments, an anti-human OX40 agonist antibody may be administered in combination with an antibody directed against VEGFR2. In some embodiments, an anti-human OX40 agonist antibody may be administered in combination with ramucirumab. In some embodiments, an anti-human OX40 agonist antibody may be administered in combination with a VEGF Receptor fusion protein. In some embodiments, an anti-human OX40 agonist antibody may be administered in combination with aflibercept. In some embodiments, an anti-human OX40 agonist antibody may be administered in combination with ziv-aflibercept (also known as VEGF Trap or Zaltrap®). In some embodiments, an anti-human OX40 agonist antibody may be administered in combination with a bispecific antibody targeting VEGF and Ang2. In some embodiments, an anti-human OX40 agonist antibody may be administered in combination with RG7221 (also known as vanucizumab). In some embodiments, an anti-human OX40 agonist antibody may be administered in combination with an angiogenesis inhibitor and a PD-1 axis binding antagonist (e.g., a PD-1 binding antagonist such as an anti-PD-1 antibody, a PD-L1 binding antagonist such as an anti-PD-L1 antibody, and a PD-L2 binding antagonist such as an anti-PD-L2 antibody). In some embodiments, an anti-human OX40 agonist antibody may be administered in combination with bevacizumab and a PD-1 axis binding antagonist (e.g., a PD-1 binding antagonist such as an anti-PD-1 antibody, a PD-L1 binding antagonist such as an anti-PD-L1 antibody, and a PD-L2 binding antagonist such as an anti-PD-L2 antibody). In some embodiments, an anti-human OX40 agonist antibody may be administered in combination with bevacizumab and MDX-1106 (nivolumab, OPDIVO). In some embodiments, an anti-human OX40 agonist antibody may be administered in combination with bevacizumab and Merck 3475 (MK-3475, pembrolizumab, KEYTRUDA). In some embodiments, an anti-human OX40 agonist antibody may be administered in combination with bevacizumab and CT-Oi 1 (Pidilizumab). In some embodiments, an anti-human OX40 agonist antibody may be administered in combination with bevacizumab and YW243.55.S70. In some embodiments, an anti-human OX40 agonist antibody may be administered in combination with bevacizumab and MPDL3280A. In some embodiments, an anti-human OX40 agonist antibody may be administered in combination with bevacizumab and MEDI4736. In some embodiments, an anti-human OX40 agonist antibody may be administered in combination with bevacizumab and MDX-1105.

In some embodiments, an anti-human OX40 agonist antibody may be administered in combination with an antineoplastic agent. In some embodiments, an anti-human OX40 agonist antibody may be administered in combination with an agent targeting CSF-1R (also known as M-CSFR or CD115). In some embodiments, an anti-human OX40 agonist antibody may be administered in combination with an anti-CSF-1R antibody (also known as IMC-CS4 or LY3022855). In some embodiments, an anti-human OX40 agonist antibody may be administered in combination with an anti-CSF-1R antibody, RG7155 (also known as R05509554 or emactuzumab). In some embodiments, an anti-human OX40 agonist antibody may be administered in combination with an interferon, for example interferon-α or interferon-γ. In some embodiments, an anti-human OX40 agonist antibody may be administered in combination with Roferon-A (also known as recombinant Interferon α-2a). In some embodiments, an anti-human OX40 agonist antibody may be administered in combination with GM-CSF (also known as recombinant human granulocyte macrophage colony stimulating factor, rhu GM-CSF, sargramostim, or Leukine®). In some embodiments, an anti-human OX40 agonist antibody may be administered in combination with IL-2 (also known as aldesleukin or Proleukin®). In some embodiments, an anti-human OX40 agonist antibody may be administered in combination with IL-12. In some embodiments, an anti-human OX40 agonist antibody may be administered in combination with IL27. In some embodiments, an anti-human OX40 agonist antibody may be administered in combination with IL-15. In some embodiments, an anti-human OX40 agonist antibody may be administered in combination with ALT-803. In some embodiments, an anti-human OX40 agonist antibody may be administered in combination with an antibody targeting CD20. In some embodiments, the antibody targeting CD20 is obinutuzumab (also known as GA101 or Gazyva®) or rituximab. In some embodiments, an anti-human OX40 agonist antibody may be administered in combination with an antibody targeting GITR. In some embodiments, the antibody targeting GITR is TRX518. In some embodiments, the antibody targeting GITR is MK04166 (Merck).

In some embodiments, an anti-human OX40 agonist antibody may be administered in combination with an inhibitor of Bruton's tyrosine kinase (BTK). In some embodiments, an anti-human OX40 agonist antibody may be administered in combination with Ibrutinib. In some embodiments, an anti-human OX40 agonist antibody may be administered in combination with an inhibitor of Isocitrate dehydrogenase 1 (IDH1) and/or Isocitrate dehydrogenase 2 (IDH2). In some embodiments, an anti-human OX40 agonist antibody may be administered in combination with AG-120 (Agios).

In some embodiments, an anti-human OX40 agonist antibody may be administered in combination with Obinutuzumab and a PD-1 axis binding antagonist (e.g., a PD-1 binding antagonist such as an anti-PD-1 antibody, a PD-L1 binding antagonist such as an anti-PD-L1 antibody, and a PD-L2 binding antagonist such as an anti-PD-L2 antibody).

In some embodiments, an anti-human OX40 agonist antibody may be administered in combination with a cancer vaccine. In some embodiments, the cancer vaccine is a peptide-based cancer vaccine. In other embodiments, the cancer vaccine is a personalized peptide vaccine. In some embodiments, the peptide-based cancer vaccine is a multivalent long peptide, a multi-peptide, a peptide cocktail, a hybrid peptide, or a peptide-pulsed dendritic cell vaccine (see, e.g., Yamada et al., Cancer Sci, 104: 14-21, 2013). In some embodiments, an anti-human OX40 agonist antibody may be administered in combination with an adjuvant. In some embodiments, an anti-human OX40 agonist antibody may be administered in combination with a treatment comprising a TLR agonist, e.g., Poly-ICLC (also known as Hiltonol®), LPS, MPL, or CpG ODN. In some embodiments, an anti-human OX40 agonist antibody may be administered in combination with tumor necrosis factor α (TNFα). In some embodiments, an anti-human OX40 agonist antibody may be administered in combination with IL-1. In some embodiments, an anti-human OX40 agonist antibody may be administered in combination with HMGB1. In some embodiments, an anti-human OX40 agonist antibody may be administered in combination with an IL-10 antagonist. In some embodiments, an anti-human OX40 agonist antibody may be administered in combination with an IL-4 antagonist. In some embodiments, an anti-human OX40 agonist antibody may be administered in combination with an IL-13 antagonist. In some embodiments, an anti-human OX40 agonist antibody may be administered in combination with an IL-17 antagonist. In some embodiments, an anti-human OX40 agonist antibody may be administered in combination with an HVEM antagonist. In some embodiments, an anti-human OX40 agonist antibody may be administered in combination with an ICOS agonist, e.g., the administration of ICOS-L, or an agonistic antibody targeting ICOS. In some embodiments, an anti-human OX40 agonist antibody may be administered in combination with a treatment targeting CX3CL1. In some embodiments, an anti-human OX40 agonist antibody may be administered in combination with a treatment targeting CXCL9. In some embodiments, an anti-human OX40 agonist antibody may be administered in combination with a treatment targeting CXCL10. In some embodiments, an anti-human OX40 agonist antibody may be administered in combination with a treatment targeting CCL5. In some embodiments, an anti-human OX40 agonist antibody may be administered in combination with an LFA-1 or ICAM1 agonist. In some embodiments, an anti-human OX40 agonist antibody may be administered in combination with a Selectin agonist.

In some embodiments, an anti-human OX40 agonist antibody may be administered in combination with an inhibitor of B-Raf. In some embodiments, an anti-human OX40 agonist antibody may be administered in combination with vemurafenib (also known as Zelboraf®). In some embodiments, an anti-human OX40 agonist antibody may be administered in combination with dabrafenib (also known as Tafinlar®). In some embodiments, an anti-human OX40 agonist antibody may be administered in combination with encorafenib (LGX818).

In some embodiments, an anti-human OX40 agonist antibody may be administered in combination with an EGFR inhibitor. In some embodiments, an anti-human OX40 agonist antibody may be administered in combination with erlotinib (also known as Tarceva®). In some embodiments, an anti-human OX40 agonist antibody may be administered in combination with an inhibitor of EGFR-T790 M. In some embodiments, an anti-human OX40 agonist antibody may be administered in combination with gefitinib. In some embodiments, an anti-human OX40 agonist antibody may be administered in combination with afatinib. In some embodiments, an anti-human OX40 agonist antibody may be administered in combination with cetuximab (also known as Erbitux®). In some embodiments, an anti-human OX40 agonist antibody may be administered in combination with panitumumab (also known as Vectibix®). In some embodiments, an anti-human OX40 agonist antibody may be administered in combination with rociletinib. In some embodiments, an anti-human OX40 agonist antibody may be administered in combination with AZD9291. In some embodiments, an anti-human OX40 agonist antibody may be administered in combination with a MEK inhibitor, such as MEK1 (also known as MAP2K1) and/or MEK2 (also known as MAP2K2). In some embodiments, an anti-human OX40 agonist antibody may be administered in combination with cobimetinib (also known as CDC-0973 or XL-518). In some embodiments, an anti-human OX40 agonist antibody may be administered in combination with trametinib (also known as Mekinist®). In some embodiments, an anti-human OX40 agonist antibody may be administered in combination with binimetinib.

In some embodiments, an anti-human OX40 agonist antibody may be administered in combination with a B-Raf inhibitor (e.g., vemurafenib or dabrafenib) and an MEK inhibitor (e.g., MEK1 and/or MEK2 (e.g., cobimetinib or trametinib)). In some embodiments, an anti-human OX40 agonist antibody may be administered in combination with an inhibitor of ERK (e.g., ERK1/2). In some embodiments, an anti-human OX40 agonist antibody may be administered in combination with GDC-0994. In some embodiments, an anti-human OX40 agonist antibody may be administered in combination with an inhibitor of B-Raf, an inhibitor of MEK, and an inhibitor of ERK1/2. In some embodiments, an anti-human OX40 agonist antibody may be administered in combination with an inhibitor of EGFR, an inhibitor of MEK, and an inhibitor of ERK1/2. In some embodiments, an anti-human OX40 agonist antibody may be administered in combination with one or more MAP kinase pathway inhibitor. In some embodiments, an anti-human OX40 agonist antibody may be administered in combination with CK127. In some embodiments, an anti-human OX40 agonist antibody may be administered in combination with an inhibitor of K-Ras.

In some embodiments, an anti-human OX40 agonist antibody may be administered in combination with an inhibitor of c-Met. In some embodiments, an anti-human OX40 agonist antibody may be administered in combination with onartuzumab (also known as MetMAb). In some embodiments, an anti-human OX40 agonist antibody may be administered in combination with an inhibitor of anaplastic lymphoma kinase (ALK). In some embodiments, an anti-human OX40 agonist antibody may be administered in combination with AF802 (also known as CH5424802 or alectinib). In some embodiments, an anti-human OX40 agonist antibody may be administered in combination with crizotinib. In some embodiments, an anti-human OX40 agonist antibody may be administered in combination with ceritinib. In some embodiments, an anti-human OX40 agonist antibody may be administered in combination with an inhibitor of phosphatidylinositol 3-kinase (PI3K). In some embodiments, an anti-human OX40 agonist antibody may be administered in combination with buparlisib (B KM-120). In some embodiments, an anti-human OX40 agonist antibody may be administered in combination with pictilisib (also known as GDC-0941). In some embodiments, an anti-human OX40 agonist antibody may be administered in combination with buparlisib (also known as B KM-120). In some embodiments, an anti-human OX40 agonist antibody may be administered in combination with perifosine (also known as KRX-0401). In some embodiments, an anti-human OX40 agonist antibody may be administered in combination with a δ-selective inhibitor of phosphatidylinositol 3-kinase (PI3K). In some embodiments, an anti-human OX40 agonist antibody may be administered in combination with idelalisib (also known as GS-1101 or CAL-101). In some embodiments, an anti-human OX40 agonist antibody may be administered in combination with taselisib (also known as GDC-0032). In some embodiments, an anti-human OX40 agonist antibody may be administered in combination with BYL-719. In some embodiments, an anti-human OX40 agonist antibody may be administered in combination with an inhibitor of Akt. In some embodiments, an anti-human OX40 agonist antibody may be administered in combination with MK2206. In some embodiments, an anti-human OX40 agonist antibody may be administered in combination with GSK690693. In some embodiments, an anti-human OX40 agonist antibody may be administered in combination with ipatasertib (also known as CDC-0068). In some embodiments, an anti-human OX40 agonist antibody may be administered in combination with an inhibitor of mTOR. In some embodiments, an anti-human OX40 agonist antibody may be administered in combination with sirolimus (also known as rapamycin). In some embodiments, an anti-human OX40 agonist antibody may be administered in combination with temsirolimus (also known as CCI-779 or Torisel®). In some embodiments, an anti-human OX40 agonist antibody may be administered in combination with everolimus (also known as RADOO1). In some embodiments, an anti-human OX40 agonist antibody may be administered in combination with ridaforolimus (also known as AP-23573, MK-8669, or deforolimus). In some embodiments, an anti-human OX40 agonist antibody may be administered in combination with OSI-027. In some embodiments, an anti-human OX40 agonist antibody may be administered in combination with AZD8055. In some embodiments, an anti-human OX40 agonist antibody may be administered in combination with INK128. In some embodiments, an anti-human OX40 agonist antibody may be administered in combination with a dual PBK/mTOR inhibitor. In some embodiments, an anti-human OX40 agonist antibody may be administered in combination with XL765. In some embodiments, an anti-human OX40 agonist antibody may be administered in combination with GDC-0980. In some embodiments, an anti-human OX40 agonist antibody may be administered in combination with BEZ235 (also known as NVP-BEZ235). In some embodiments, an anti-human OX40 agonist antibody may be administered in combination with BGT226. In some embodiments, an anti-human OX40 agonist antibody may be administered in combination with GSK2126458. In some embodiments, an anti-human OX40 agonist antibody may be administered in combination with PF-04691502. In some embodiments, an anti-human OX40 agonist antibody may be administered in combination with PF-05212384 (also known as PKI-587).

In some embodiments, an anti-human OX40 agonist antibody may be administered in combination with an agent that selectively degrades the estrogen receptor. In some embodiments, an anti-human OX40 agonist antibody may be administered in combination with GDC-0927. In some embodiments, an anti-human OX40 agonist antibody may be administered in combination with an inhibitor of HER3. In some embodiments, an anti-human OX40 agonist antibody may be administered in combination with duligotuzumab. In some embodiments, an anti-human OX40 agonist antibody may be administered in combination with an inhibitor of LSD1. In some embodiments, an anti-human OX40 agonist antibody may be administered in combination with an inhibitor of MDM2. In some embodiments, an anti-human OX40 agonist antibody may be administered in combination with an inhibitor of BCL2. In some embodiments, an anti-human OX40 agonist antibody may be administered in combination with venetoclax. In some embodiments, an anti-human OX40 agonist antibody may be administered in combination with an inhibitor of CHK1. In some embodiments, an anti-human OX40 agonist antibody may be administered in combination with CDC-0575. In some embodiments, an anti-human OX40 agonist antibody may be administered in combination with an inhibitor of activated hedgehog signaling pathway. In some embodiments, an anti-human OX40 agonist antibody may be administered in combination with ERIVEDGE.

In some embodiments, an anti-human OX40 agonist antibody may be administered in combination with a radiation therapy. In some embodiments, an anti-human OX40 agonist antibody may be administered in combination with gemcitabine. In some embodiments, an anti-human OX40 agonist antibody may be administered in combination with nab-paclitaxel (ABRAXANE). In some embodiments, an anti-human OX40 agonist antibody may be administered in combination with trastuzumab. In some embodiments, an anti-human OX40 agonist antibody may be administered in combination with TVEC. In some embodiments, an anti-human OX40 agonist antibody may be administered in combination with IL27. In some embodiments, an anti-human OX40 agonist antibody may be administered in combination with cyclophosphamide. In some embodiments, an anti-human OX40 agonist antibody may be administered in combination with an agent that recruits T cells to the tumor. In some embodiments, an anti-human OX40 agonist antibody may be administered in combination with lirilumab (IPH2102/BMS-986015). In some embodiments, an anti-human OX40 agonist antibody may be administered in combination with Idelalisib. In some embodiments, an anti-human OX40 agonist antibody may be administered in combination with an antibody that targets CD3 and CD20. In some embodiments, an anti-human OX40 agonist antibody may be administered in combination with REGN1979. In some embodiments, an anti-human OX40 agonist antibody may be administered in combination with an antibody that targets CD3 and CD19. In some embodiments, an anti-human OX40 agonist antibody may be administered in combination with blinatumomab.

In some embodiments, an anti-human OX40 agonist antibody may be administered in conjunction with an oncolytic virus. In some embodiments, an anti-human OX40 agonist antibody may be administered in conjunction with carboplatin and nab-paclitaxel. In some embodiments, an anti-human OX40 agonist antibody may be administered in conjunction with carboplatin and paclitaxel. In some embodiments, an anti-human OX40 agonist antibody may be administered in conjunction with cisplatin and pemetrexed. In some embodiments, an anti-human OX40 agonist antibody may be administered in conjunction with cisplatin and gemcitabine. In some embodiments, an anti-human OX40 agonist antibody may be administered in conjunction with FOLFOX. In some embodiments, an anti-human OX40 agonist antibody may be administered in conjunction with FOLFIRI.

Such combination therapies noted above comprise both combined administration (where two or more therapeutic agents are included in the same or separate formulations) and separate administrations, in which case, the administration of the antibody described in the present invention can occur prior to, simultaneously, and/or following, the administration of an additional therapeutic agent and/or adjuvant. Antibodies of the invention can also be used in combination with a radiation therapy.

An antibody of the invention (and any additional therapeutic agent) can be administered by any suitable means, including a parenteral, intrapulmonary, and intranasal, and, if desired for local treatment, an intralesional administration. Parenteral infusions include intramuscular, intravenous, intraarterial, intraperitoneal, or subcutaneous administration. The dosing can be made by any suitable routes of administration, e.g. by injections, such as intravenous or subcutaneous injections, partly depending on whether the administration is brief or chronic. Various dosing schedules provided herein include, but not limited to, single or multiple administrations (e.g. bolus administration) and pulse infusions over various time-points.

Antibodies of the invention would be formulated, dosed, and administered in a fashion consistent with good medical practice. Factors for consideration in this context include the particular disorder being treated, the particular mammal being treated, the clinical condition of each individual patient, the cause of an disorder, the delivery site of the pharmaceutical composition, the method of administration, the scheduling of administration, and other factors known to the medical practitioners. The antibody may, but needs not to, be formulated with one or more agents currently used for the prevention or treatment of the disorder in question. The effective amount of such other agents depends on the amount of antibody present in the formulation, the type of disorder and treatment, and other factors discussed above. These other agents are generally used with the same dosages and administration routes as described herein, or about from 1% to 99% of the dosages described herein, or with any dosage and administration route that is empirically/clinically considered to be appropriate.

For the prevention or treatment of a disease, the appropriate dosage of an antibody described in the present invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to an antibody, and the discretion of an attending physician. The antibody could be administered to a patient at one time or over a series of administrations. Depending on the type and severity of the disease, about 1 μg/kg to 40 mg/kg of antibody can be an initial candidate dosage, whether, for example, by one or more separate administrations, or by a continuous infusion to a patient. A typical daily dosage may range from about 1 μg/kg to 100 mg/kg or more, depending on the factors mentioned above. For repeated administrations over several days or longer, depending on the condition, the treatment would generally be sustained until a desired outcome is achieved, e.g. the suppression of disease progression. The pharmaceutical composition may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The therapeutic progress is easily monitored by conventional techniques and methods.

It is understood that any of the above formulations or therapeutic methods may be carried out using an immunoconjugate of the invention in place of or in addition to an anti-OX40 antibody.

In certain embodiments, an intravenous (IV) infusion of an anti-OX40 antibody as disclosed herein is administered to a patient every 3, 4, 5, 6, 7, or 8 weeks. The dose of the anti-OX40 antibody per patient may be 5 mg, 10 mg, 15 mg, 30 mg, 50 mg, 100 mg, 150 mg, 200 mg, 300 mg, or 500 mg. In certain embodiments, the patient may have renal cell carcinoma, hepatocellular carcinoma, or head and neck squamous cell carcinoma.

In certain embodiments, an anti-OX40 antibody is administered to a patient at a dose and dosing regimen to achieve one or more of the following desirable changes to immune cells in the blood and the tumor microenvironment (TME) of said patient, for example:
An increase in the percentage of proliferation (e.g., Ki67) and activation (e.g., CD69, CD25) markers on the CD4⁺ or CD8⁺ T cells from the peripheral blood of the patient measured by flow cytometry;
OX40 receptor levels and occupancy level of the anti-OX40 antibody on CD4⁺CD25⁺ T cells from the peripheral blood of the patient measured by flow cytometry; and
Target engagement (e.g., OX40 downregulation), CD8⁺ and CD4⁺ T cells proliferation (e.g., Ki67 upregulation) measured by multiplexed immunohistochemistry (mIHC) or multiplexed immunofluorescence (mIF) on matched pre- and post-treatment tumor biopsy tissues from the patient.

In certain embodiments, the dose and dosing frequency of an anti-OX40 antibody herein are selected or determined to ensure an exposure profile with sufficient peak-to-trough ratio and the least accumulation upon repeated dosing in a patient, thereby preventing prolonged receptor saturation and subsequent T-cell exhaustion so as to achieve sustained immune-mediated anti-tumor activity in the patient.

In other embodiments, the dose and/or dosing frequency of the anti-OX40 antibody herein are selected or determined by the exposure profile together with the pharmacodynamic (PD) findings, such as the desired changes in the patient's immune cells and/or TME as described above.

### EXAMPLES

### Example 1 Preparation of Anti-OX40 Antibody HFB10-1E1hG1

The heavy chain and light chain coding nucleotide sequences (SEQ ID NO: 24 and 32) of the anti-OX40 antibody HFB10-1E1hG1 were synthesized (JinWeizhi Company), and respectively cloned into the pFUSE vector. The plasmids containing the heavy chain and the light chain nucleotide coding sequences were transiently co-transfected into the 293F suspension cells using PEI at a ratio of 1:1 to express the full-length antibody. After 1 week of incubation, the antibody was purified on the AKTA system using the Superdex™ 200 Increase prepacked column.

### Example 2 Binding Properties of HFB10-1E1hG1

### Example 2.1 Binding Properties of HFB10-1E1hG1 and OX40 Protein

For the pre-test ELISA, a 96-well plate was coated overnight with 5 μg/ml anti-OX40 antibody, including Reference 1, Reference 2, Reference 3, Reference 4, HFB10-1E1hG1, and isotype control. The next day, the plate was blocked with 1% BSA (Sangon Biotech, catalog number A600332-0100) in PBST at 37° C. for 2 hours, and then a defined concentration of biotinylated OX40 recombinant protein was added.

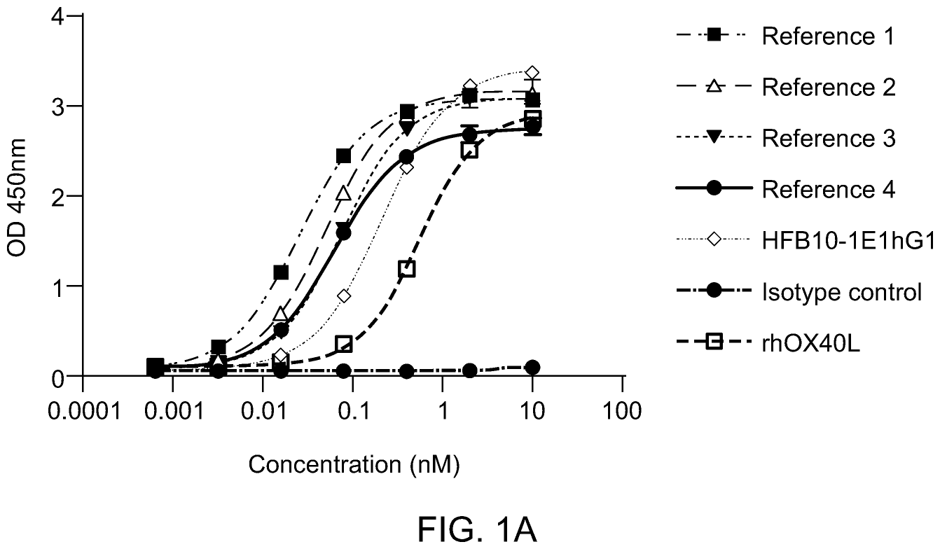

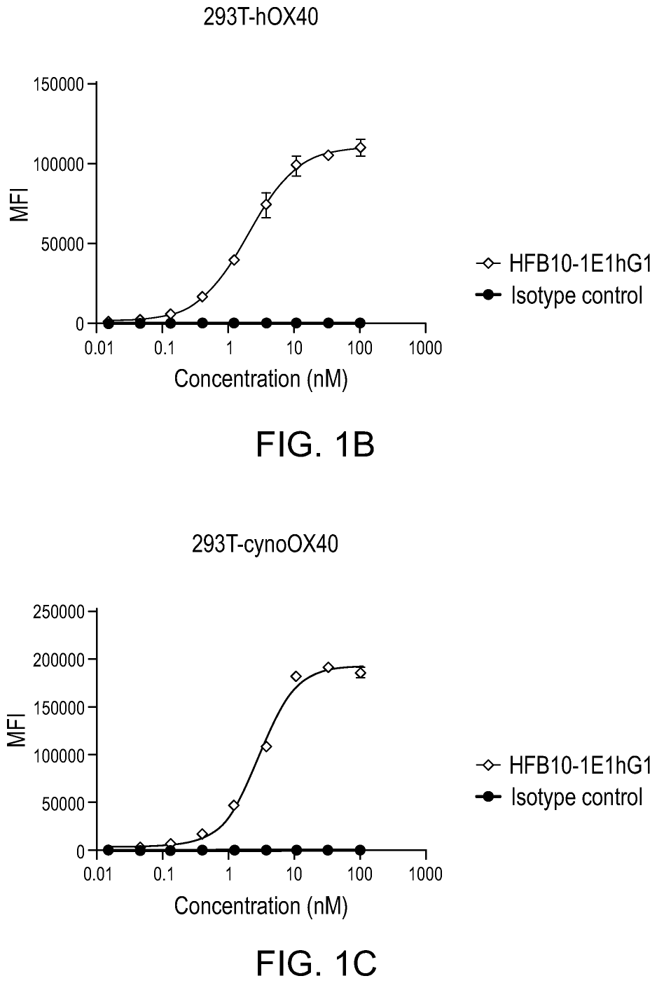

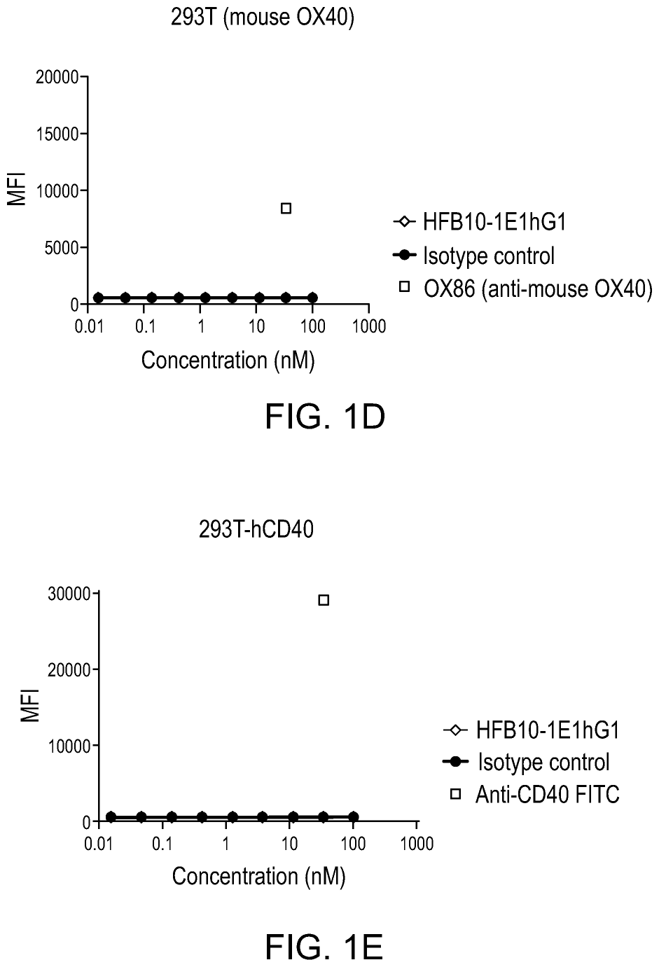

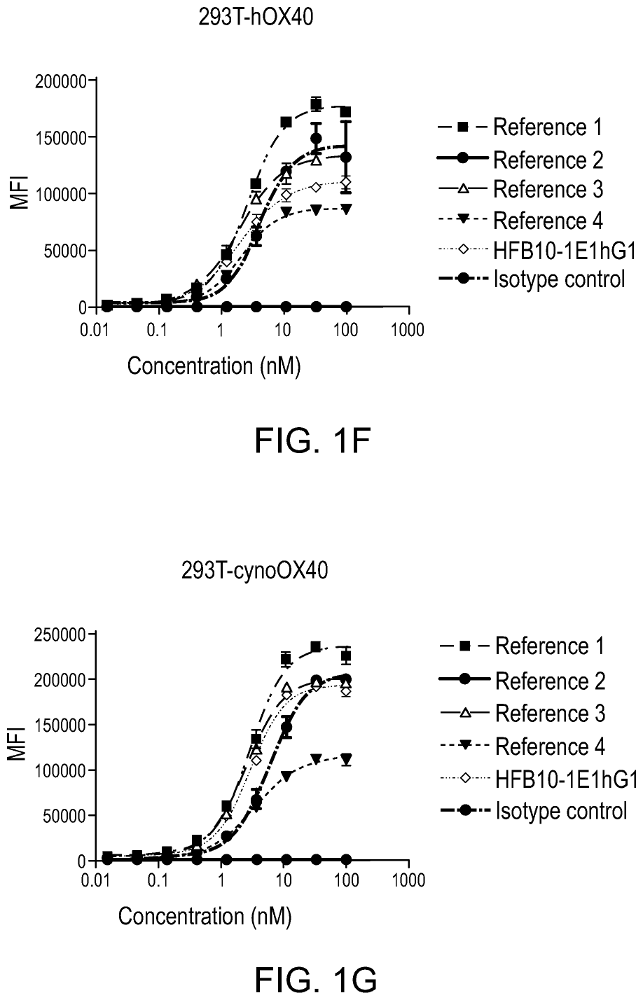

The EC80 was measured based on the binding of biotinylated OX40 recombinant protein to the anti-OX40 antibody. The EC80 of Reference 1 is 0.09 nM; the EC80 of Reference 2 is 0.22 nM; the EC80 of Reference 3 is 0.16 nM; the EC80 of Reference 4 is 0.24 nM; the EC80 of HFB10-1E1hG1 is 0.71 nM; the EC80 of OX40L is 1.6 nM. As shown in FIG. 1A.

### Example 2.2 Binding Properties of HFB10-1E1hG1 and the OX40 Protein Expressed on the Surface of 293T Cells

For overexpression of OX40, including human-OX40 (Sinobiological, Cat. HG10481-UT), cynomolgus monkey-OX40 (Sinobiological, Cat. CG90846-UT), mouse-OX40 (Sinobiological, Cat. MG50808-UT) or human CD40 (Sinobiological, Cat. HG10774-UT), the DNA plasmids encoding these targets were transiently transfected into 293T cells according to the instructions of Lipusectamine LTX Reagent and PLUS Reagent (Thermo, Cat. 15338100). The 293T cells were harvested for use at 48 hours post transfection. To determine the binding affinity, the harvested cells were incubated with a defined concentration of primary antibody HFB10-1E1hG1 at 4° C. for 1 hour. Then, after washing twice with PBS, the cells were incubated with the secondary goat anti-human IgG PE antibody (1:200; Abcam, Cat. ab98596) at room temperature for 30 minutes. Detection of the HFB10-1E1hG1 binding was performed by Beckman CytoFLEX flow cytometer.

The EC50 of the binding of HFB10-1E1hG1 to the human OX40 protein expressed on the surface of 293T cells is 2 nM (MFI), as shown in FIG. 1B.

The EC50 of the binding of HFB10-1E1hG1 to the cynomolgus monkey OX40 protein expressed on the surface of 293T cells is 2.9 nM (MFI), as shown in FIG. 1C.

HFB10-1E1hG1 does not bind to the mouse OX40 protein (MFI) expressed on the surface of 293T cells, as shown in FIG. 1D.

HFB10-1E1hG1 does not bind to the human CD40 protein (MFI) expressed on the surface of 293T cells, as shown in FIG. 1E.

The EC50s of the binding of HFB10-1E1hG1, Reference 1, Reference 2, Reference 3, and Reference 4 to the human OX40 protein expressed on the surface of 293T cells are shown in FIG. 1F. The EC50 of HFB10-1E1hG1 is 2.02 nM (MFI); the EC50 of Reference 1 is 2.67 nM (MFI); the EC50 of Reference 2 is 4.17 nM (MFI); the EC50 of Reference 3 is 1.92 nM (MFI); the EC50 of Reference 4 is 2.11 nM (MFI).

The EC50s of the binding of HFB10-1E1hG1, Reference 1, Reference 2, Reference 3, and Reference 4 to the cynomolgus monkey OX40 protein expressed on the surface of 293T cells are shown in FIG. 1G. The EC50 of HFB10-1E1hG1 is 2.94 nM (MFI); the EC50 of Reference 1 is 2.91 nM (MFI); the EC50 of Reference 2 is 6.13 nM (MFI); the EC50 of Reference 3 is 2.57 nM (MFI); the EC50 of Reference 4 is 3.54 nM (MFI).

### Example 3 Agonistic Activity of HFB10-1E1hG1

### Example 3.1 Agonistic Activity of HFB10-1E1hG1 in Jurkat Reporter Cells

The anti-OX40 antibodies can be divided into two classes according to their mode of action. The first class of OX40 agonistic activity does not depend on the cross-linking of Fc receptors, while the other class requires Fc receptor cross-linking to have OX40 agonistic activity. Tumor tissue and the surrounding draining lymph nodes have more existing tumor-related inflammatory cells, and Fc receptor FcγR2b has more aggregation around the tumor cells. Therefore, the “cross-linked antibody” agonists have greater tissue selectivity. These antibody agonists produce obvious agonistic effects only in the tumor microenvironment and maintain at a low level in normal body tissue, therefore, increase the treatment safety.

To determine the agonistic activity of HFB10-1E1hG1, a 96-well plate was coated with 5 μg/ml Fc specific anti-human IgG antibody (Sigma, Cat. SAB3701275) overnight. Recombinant Jurkat reporter cells expressing the GFP gene under the control of the NF-kb response element with constitutive expression of human OX40 were used. A defined concentration of HFB10-1E1hG1 and 1×10⁵ Jurkat reporter cells were added to one well. In another experiment, 50 nM OX40L (Acrobiosystems, Cat. OXL-H52Q8) was added together with HFB10-1E1hG1 to determine the cooperative effect. After 24 hours of incubation, Jurkat reporter cells were harvested, and the agonistic activity of HFB10-1E1hG1 in Jurkat reporter cells was measured by GFP positive signal using a Beckman CytoFLEX flow cytometer.

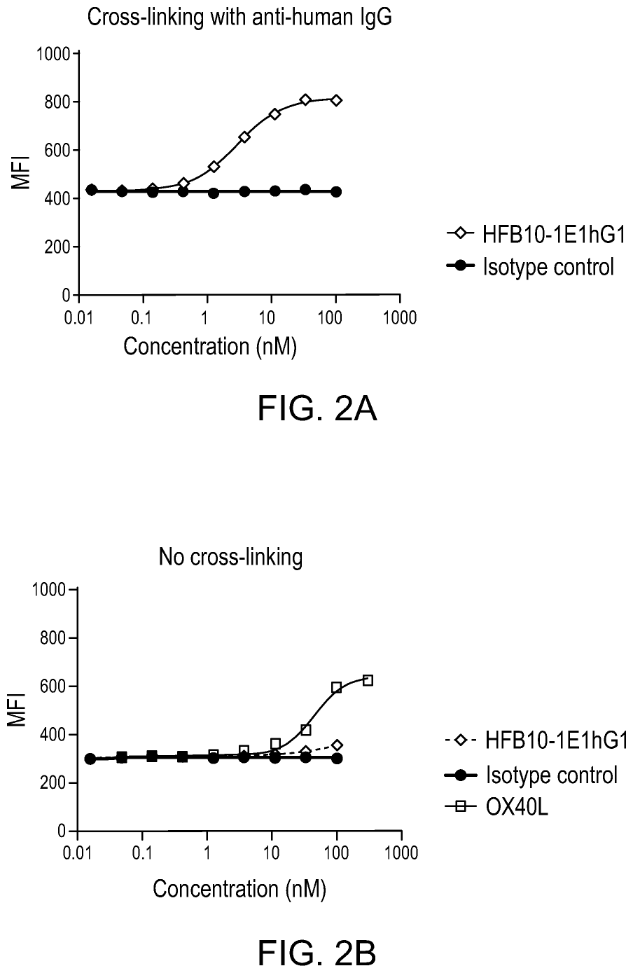

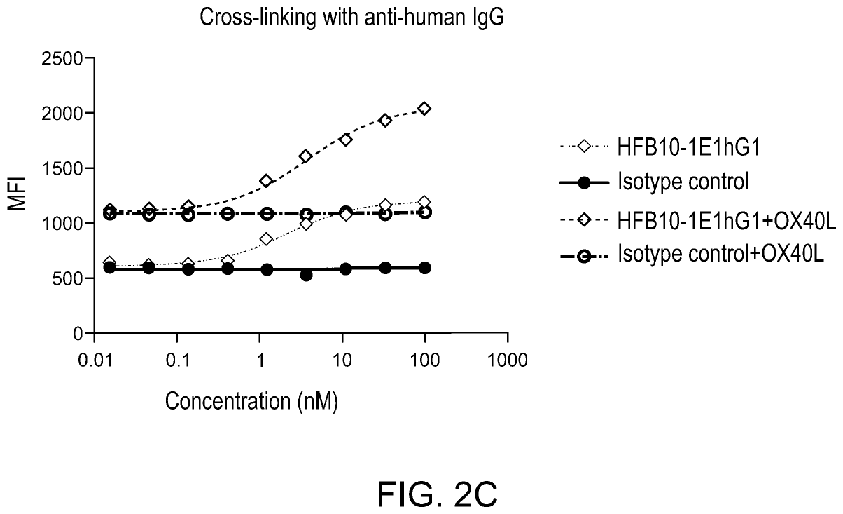

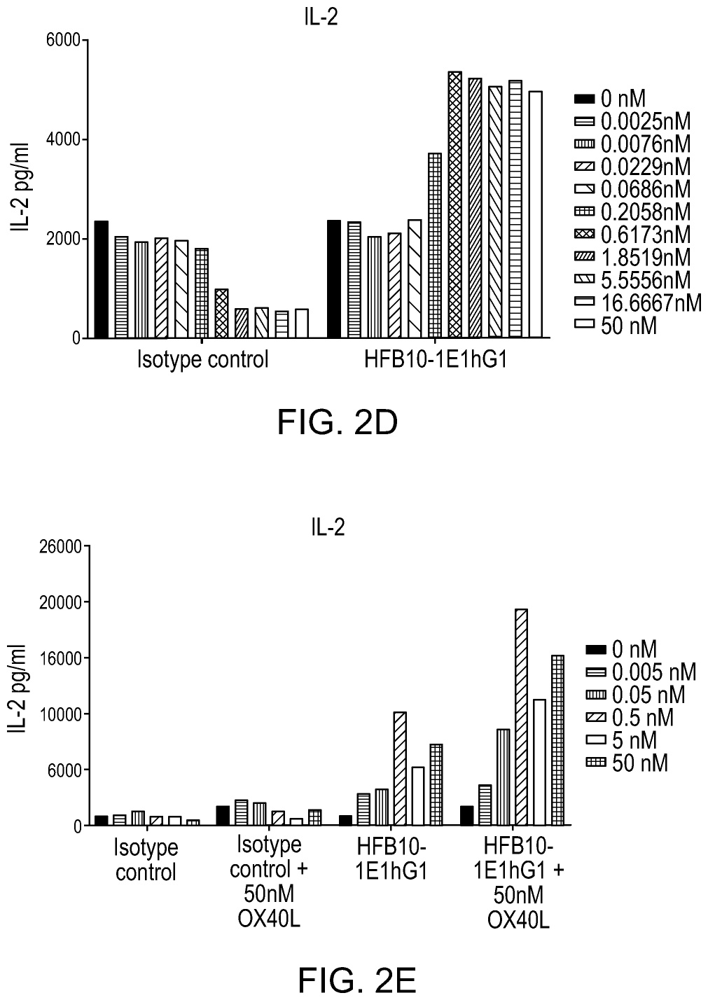

The agonistic activity of HFB10-1E1hG1 is anti-human IgG cross-linking dependent. When cross-linked with anti-human IgG, the EC50 of HFB10-1E1hG1 is 2.9 nM (MFI of GFP), as shown in FIG. 2A. In the absence of anti-human IgG cross-linkage, the EC50 of HFB10-1E1hG1 is much greater, and the trimeric OX40L recombinant protein alone can activate the NF-kb signaling with EC50=45 nM (GFP MFI), as shown in FIG. 2B.

When cross-linked with anti-human IgG, HFB10-1E1hG1 showed a synergistic agonistic effect with OX40L. The combination of HFB10-1E1hG1 and OX40L together increased the MFI of GFP compared to each individual components acting alone, as shown in FIG. 2C.

### Example 3.2 Agonistic Activity of HFB10-1E1hG1 in the Primary CD4⁺ T Cells

In order to determine the agonistic activity of HFB10-1E1hG1 in the primary CD4⁺ T cells, a 96-well plate was coated with 0.3 μg/ml or 1 μg/ml anti-CD3 antibody (Thermo, Cat. 16-0037-81) and 5 μg/ml Fc specific anti-human IgG antibody (Sigma, Cat. SAB3701275) overnight. The next day, the primary CD4⁺ T cells were harvested according to the instructions of the CD4⁺ T cell isolation kit (Miltenyi, Cat. 130-045-101). A defined concentration of HFB10-1E1hG1 and 2 μg/ml anti-CD28 antibody (Thermo, Cat. 16-0289-81) were added together with 1×10⁵ primary CD4⁺ T cells into one well. In another experiment, 50 nM OX40L (Acrobiosystems, Cat. OXL-H52Q8) was added together with HFB10-1E1hG1 to determine the synergistic effect between HFB10-1E1hG1 and OX40L. After 3 days of incubation, the IL-2 secretion level in the supernatant was measured to evaluate the agonistic activity of HFB10-1E1hG1 in the primary CD4⁺ T cells according to the instructions of the human IL-2 DuoSet ELISA kit (R&D, Cat. DY202-05).

To determine the agonistic activity of HFB10-1E1hG1 in the primary CD4⁺ T cells, the purified CD4⁺ T cells were preactivated with 1 μg/ml anti-CD3 antibody and 2 μg/ml anti-CD28 antibody. The plate was pre-coated with 5 μg/ml anti-human IgG to promote its cross-linkage with HFB10-1E1hG1. After three days of incubation, the agonistic activity of HFB10-1E1hG1 in primary CD4⁺ T cells was measured by IL-2 secretion, and the obtained EC50 was 0.2 nM, as shown in FIG. 2D.

In primary CD4⁺ T cells, HFB10-1E1hG1 showed a synergistic agonistic effect with OX40L. Incubation of HFB10-1E1hG1 with 50 nM OX40L enhanced the secretion of IL-2 compared to the individual components acting alone, as shown in FIG. 2E.

### Example 4 Pharmacokinetic Test of HFB10-1E1hG1

A 384-well plate was coated with 1 μg/ml of F(ab′)2 (Fc specific) goat anti-human IgG antibody (Jackson I R, Cat. 109-006-098) in 30 μl/well PBS overnight. After washing three times with PBST buffer, the plate was blocked with a blocking buffer containing 1 mM EDTA, 0.05% Tween, and 2% BSA in PBS at 37° C. for 1 hour. Then, starting from 1/150, a three-fold serial dilution of mouse serum samples was added at 15 μL/well, and the plate was incubated at 37° C. for 2 hours. After washing three times with PBST buffer, a secondary peroxidase-goat anti-human IgG antibody (Jackson I R, Cat. 109-035-003) was added at 1/5000 dilution and incubated at 37° C. for half an hour. TMB substrate (Biolegend, Cat. 421101) was added and incubated for another 15 minutes. Finally, ELISA stop solution (Beijing Dingguo Changsheng Biotechnology Co., Ltd.) was added, and the plate was read at 450 nm with Multiskan Sky Microplate Spectrophotometer (ThermoFisher).

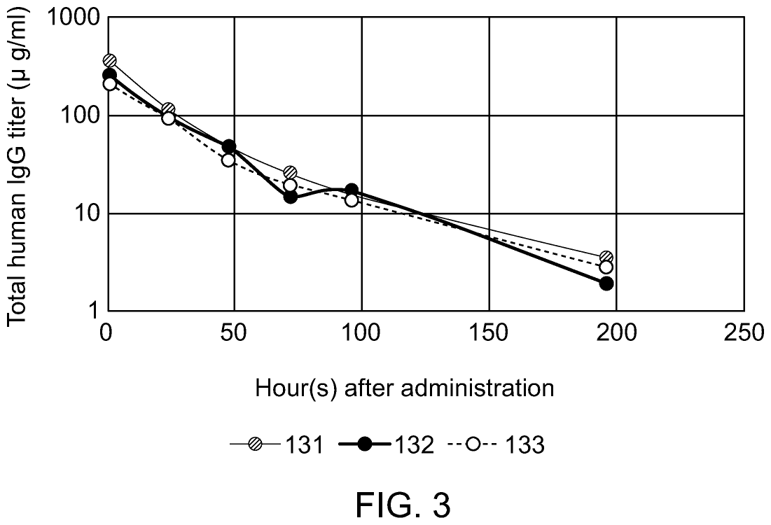

The hOX40 knock-in mice (131, 132, 133, purchased from Shanghai Southern Model Biology Research Center), was administered intravenously with 10 mg/kg HFB10-1E1hG1. Mouse serum was collected at 1 hour, 24 hours, 48 hours, 72 hours, 96 hours, and 196 hours post administration. The half-life of HFB10-1E1hG1 in hOX40-KI mouse serum was determined to be 24 hours, as shown in FIG. 3. The data points at hour 0 are 131, 132, and 133 from top to bottom.

### Example 5 In Vivo Anti-Tumor Efficacy of HFB10-1E1hG1

hOX40 knock-in mice were purchased from Shanghai Southern Model Biology Research Center. After 5 days of isolation, each mouse was subcutaneously inoculated with 8×10⁵ MC38 tumor cells (provided by Professor Zhang Hongkai of Nankai University) in 100 μl PBS. When the tumor size reached 70-100 mm³, anti-OX40 antibody treatment was initiated by intraperitoneal administration of anti-OX40 antibody to mice at 10 mg/kg in 100 μl PBS and Q3D×5. The tumor size and mouse body weight were measured twice a week. Both long and short diameters of each tumor are measured by caliper. The tumor volume in mm³ was calculated using the following formula: V=0.5a×b2, where a and b each represents the long and short diameter of the tumor.

Day 7: 35 8-week-old mice were inoculated with MC38 tumor cell
Obtained MC38 cells from Professor Zhang Hongkai
Inoculated 8×10⁵ MC38 cells per mouse

Day 0: The average tumor size was 75 mm³ (40-120 mm³)
5 mice per group, 4 groups:
PBS
Reference antibody 1
HFB10-1E1hG1
Reference antibody 2
initiated by intraperitoneal administration of 10 mg/kg Q3D×5 antibody.

Day 18: The tumor size in the PBS control group reached 2000 mm³. The mice were sacrificed.
Tissues: blood, LN, liver, spleen, tumor
Phenotypic analysis: T cell CD3/CD4/CD8; Treg CD4/CD25/Foxp3; NK CD3/CD16/CD56

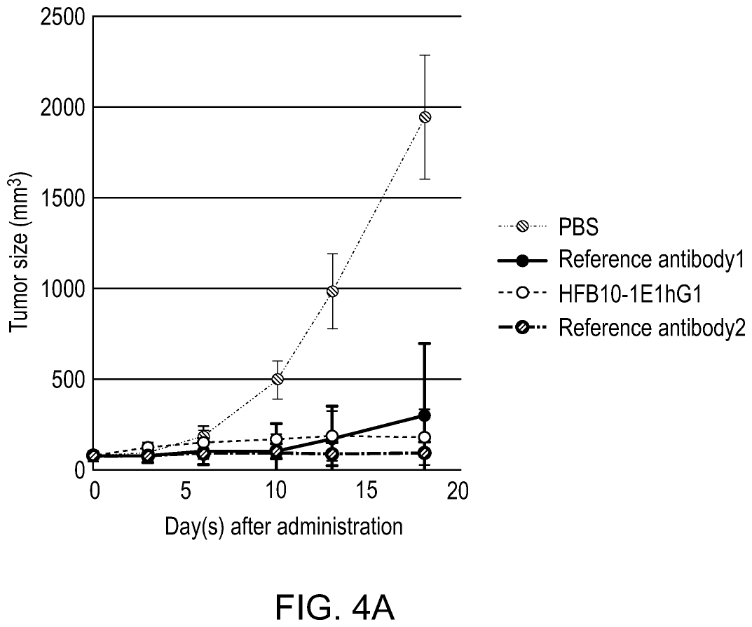

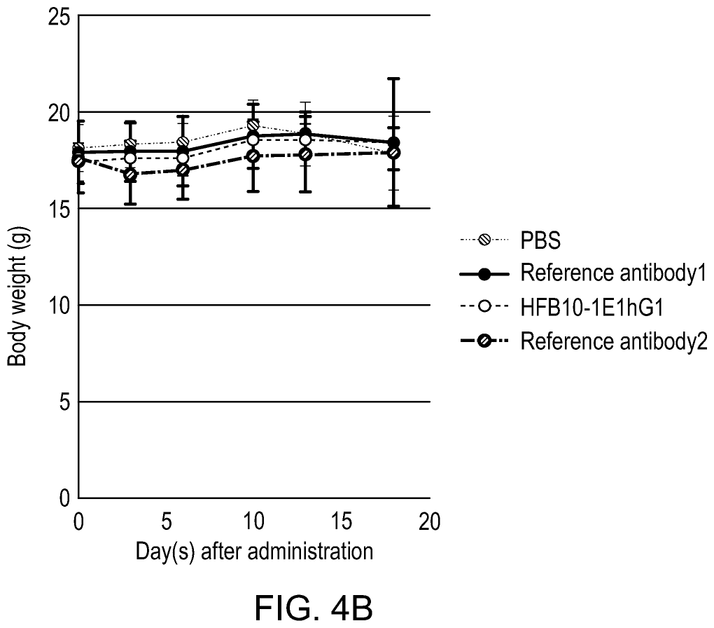

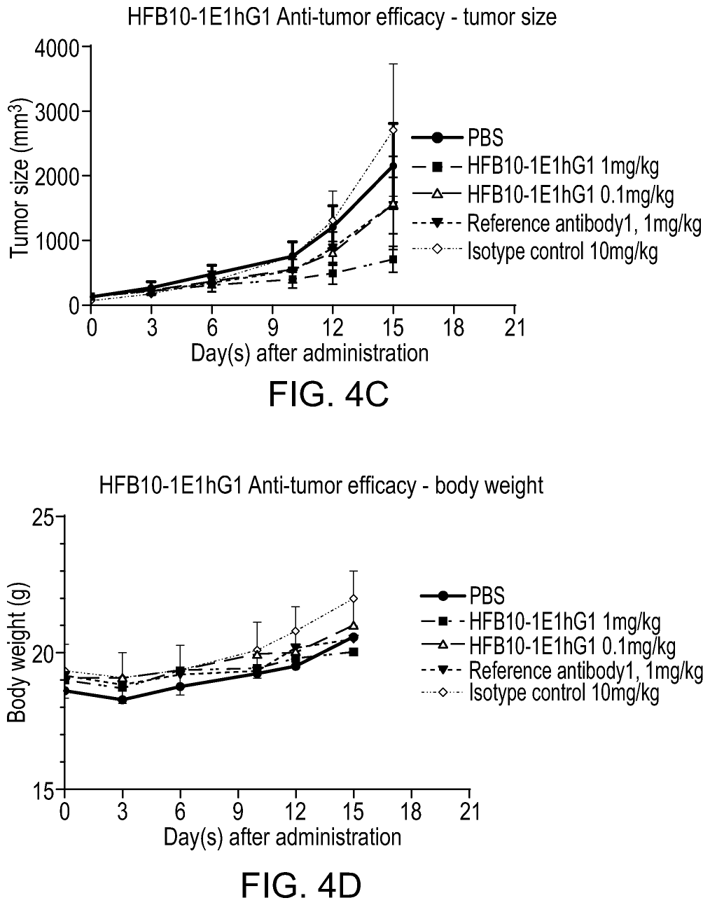

Experiments showed that HFB10-1E1hG1 significantly inhibited tumor growth compared to the PBS control without causing the side effects featured by the decrease of body weight, as shown in FIG. 4A and FIG. 4B. In FIG. 4A, the rightmost data points from top to bottom were PBS, Reference antibody 1, HFB10-1E1hG1, and Reference antibody 2. In FIG. 4B, from left to right, the second set of data from top to bottom were PBS, Reference antibody 1, Reference antibody 2, and HFB10-1E1hG1.

The efficacy of antibody dose-response was performed on the hOX40 knock-in mice. The information for each treatment group was as follows:
Group 1: PBS: Q3D×5, intraperitoneal;
Group 2: HFB10-1E1hG1: 1 mg/kg, intraperitoneal, Q3/4D×5, intraperitoneal;
Group 3: HFB10-1E1hG1: 0.1 mg/kg, intraperitoneal, Q3/4D×5, intraperitoneal;
Group 4: Reference antibody: 1: 1 mg/kg, intraperitoneal, Q3D×5, intraperitoneal.

A total of 20 hOX40 knock-in mice, 4 groups, 5 mice per treatment group.

Mice were inoculated with MC38 tumor cells. Starting when the average tumor size was 75 mm³, a total 5 antibody injections were performed on day 0, day 3, day 6, day 10, and day 13. The tumor size and mouse body weight were measured twice a week for up to three weeks or until the tumor size was greater than 2000 mm³.

The tumor size and body weight records under different doses of HFB10-1E1hG1 treatment were shown in FIG. 4C and FIG. 4D. The results suggested that treatment with 1 mg/kg of HFB10-1E1hG1 exhibited the best anti-tumor efficacy in vivo.

### Example 6 In Vitro Characterization of HFB10-1E1hG1

Example 6.1 Accelerated stability assessment of HFB10-1E1hG1 The antibody sample (HFB10-1E1hG1, 3.1 mg/mL, lot #CP181130004) was concentrated to 10 mg/mL (lot #JW20181203, lot #20190624) through a centrifugal filtration device (Millipore, Cat #UFC503096). An appropriate amount of concentrated antibody was transferred to a clean 600 μl tube and incubated at 25° C. and 40° C. for 7, 14, and 30 days, respectively.

The incubated samples were analyzed by SEC-HPLC and SDS-PAGE:

For SEC-HPLC, 50 μg of each treated sample was injected and run at a flow rate of 0.7 mL/min, 40 minutes/test in 1×PBS buffer, pH 7.4 (diluted by using Milli-Q pure water from 10×PBS buffer (Sangon, Cat #E607016)-0500), and the absorbance was detected at UV 280 nm (untreated samples stored at 4° C. were also loaded for analysis). The results were shown in Table 1. After incubating at 25° C. and 40° C. for up to 30 days, no significant increase in the aggregation or degradation peak of HFB10-1E1hG1 was observed on the SEC curve, indicating that HFB10-1E1hG1 has good stability under the treatment conditions. The slight degradation observed after 30 days of incubation at 40° C. may indicate the instability of prolonged incubation at high temperatures.

TABLE 1
SEC analysis-temperature

| | | | | Small fragment |
|---|---|---|---|---|
| Antibody name | Condition(s) | Aggregation % | monomer | (s) % |
| HFB10-1E1hG1 | Stored at 4° C. | 1.70 | 98.31 | 0 |
| | 25° C., 7 days | 2.01 | 97.99 | 0 |
| | 25° C., 14 days | 1.96 | 98.04 | 0 |
| | 25° C., 30 days | 2.06 | 97.94 | 0 |
| | 40° C., 7 days | 3.99 | 96.00 | 0 |
| | 40° C., 14 days | 3.75 | 96.10 | 0 |
| | 40° C., 30 days | 3.6 | 95.18 | 1.22 |

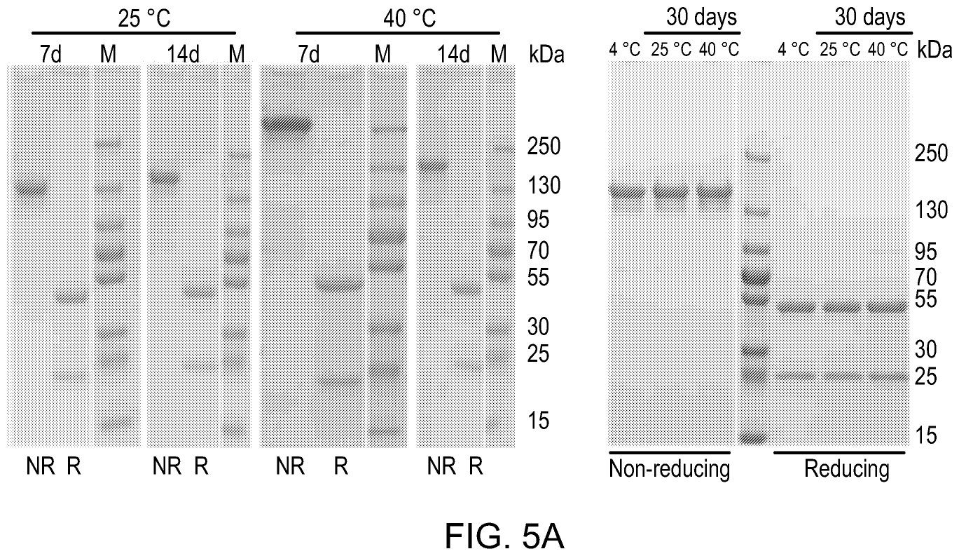

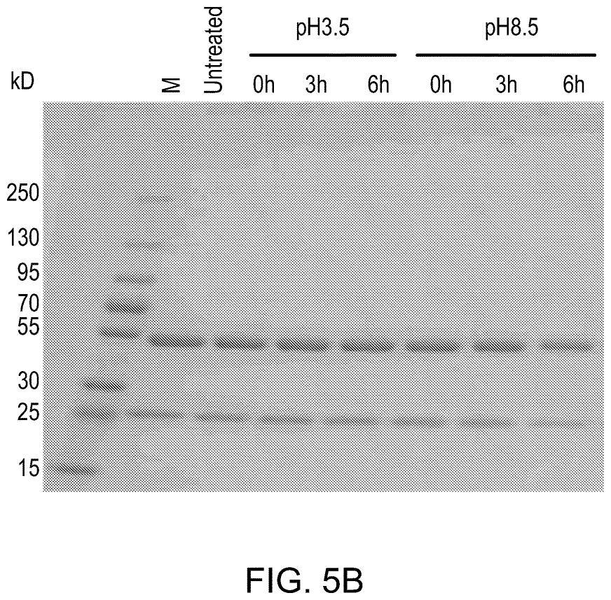

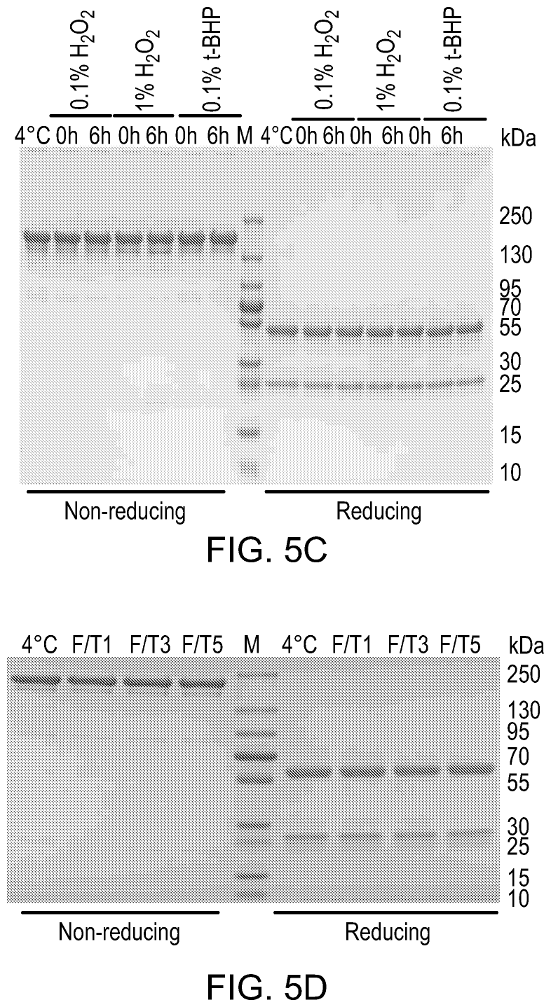

4 μg of each treated sample was loaded onto a 4%-20% gradient gel under non-reducing and reducing conditions for SDS-PAGE. The gel was run in a tris-glycine buffer at 150V for 1 hour and stained in the staining solution (TaKaRa, Cat #T9320A) for more than 1 hour. It was then decolorized several times in distilled water and imaged on a white light plate (also untreated samples stored at 4° C. were used for analysis), as shown in FIG. 5A.

After 30 days of incubation at 25° C. and 40° C., no obvious aggregated or degradated bands of HFB10-1E1hG1 were observed on the SDS-PAGE image, indicating that HFB10-1E1hG1 has good stability under such treatment conditions; after 30 days incubation at 40° C., slightly degradated bands on the non-reducing gel and the aggregated band on the reducing gel may indicate the instability of the prolonged incubation of HFB10-1E1hG1 at high temperature.

### Example 6.2 Degradation Experiment of HFB10-1E1hG1

### 1. Low pH Stress Test:

An appropriate amount of antibody (HFB10-1E1hG1, 3.1 mg/mL) was transferred to a 600 μl tube, and then 2 M acetic acid was added at a ratio of 1:20 (v/v, acid relative to the antibody sample, the final pH was adjusted to about 3.5). The sample was mixed thoroughly and incubated at room temperature for 0, 3, or 6 hours. After incubation, the pH of the antibody solution was adjusted to 7.4 with neutralization buffer (1 M Tris-HCl, pH 9.0 was added to the incubation sample, at a ratio of 13:100, v/v). The samples were analyzed by SEC-HPLC and SDS-PAGE as previously described (see accelerated stability experiment, note: untreated samples stored at 4° C. were also loaded for analysis).

### 2. High pH Stress Test:

An appropriate amount of antibody (HFB10-1E1hG1, 3.1 mg/mL) was transferred to a 600 μl tube, and then 1 M pH 8.5 Tris-HCl was added at a ratio of 1:25 (v/v, the final pH was adjusted to about 8.5). The samples were mixed thoroughly and incubated at room temperature for 0 or 6 hours. The samples were analyzed by SEC-HPLC and SDS-PAGE (analysis of reducing conditions only) as previously described.

SEC analysis is shown in Table 2. After incubating in the corresponding pH 3.5 and pH 8.5 solutions for up to 6 hours, no increase in the aggregation or degradation peak of HFB10-1E1hG1 was observed on the SEC curve, indicating that the HFB10-1E1hG1 has good stability under such treatment conditions.

TABLE 2
SEC analysis-pH

| | | | | Small fragment |
|---|---|---|---|---|
| Antibody name | Condition(s) | Aggregation % | Monomer % | (s) % |
| HFB10-1E1hG1 | Stored at 4° C. | 1.47 | 97.57 | 0.96 |
| | pH3.5, 0 h | 1.21 | 97.49 | 1.3 |
| | pH3.5, 3 h | 1.15 | 97.63 | 1.22 |
| | pH3.5, 6 h | 1.25 | 97.62 | 1.13 |
| | pH8.5, 0 h | 2.16 | 97.48 | 0.37 |
| | pH8.5, 6 h | 2.11 | 97.53 | 0.36 |

The SDS-PAGE analysis is shown in FIG. 5B. After incubating in low pH and high pH buffers for up to 6 hours, no change in HFB10-1E1hG1 was observed on the SDS-PAGE gel, indicating that the HFB10-1E1hG1 has good stability under such treatment conditions.

### Example 6.3 Oxidative Stress Experiment of HFB10-1E1hG1

An appropriate amount of antibody (HFB10-1E1hG1, 3.1 mg/mL) was transferred to a 600 μl tube, and then H₂O₂ (0.1% and 1%, respectively) or t-BHP (to the final 0.1%) was added. After mixed thoroughly and incubated at room temperature for 0 or 6 hours, the samples were analyzed by SEC-HPLC and SDS-PAGE as previously described.

Note: 1) untreated samples stored at 4° C. were loaded for analysis; 2) SEC running buffer was prepared, as 100 mM NaH2PO4, 150 mM NaCl, pH 6.8.

SEC analysis was shown in Table 3. After incubating in the corresponding 0.1% H₂O₂, 1% H₂O₂, and 0.1% t-BHP (tert-butyl hydroperoxide) solution for 6 hours, no significant HFB10-1E1hG1 changes were observed on the SEC curve, indicating that the HFB10-1E1hG1 has good stability under these treatment conditions.

TABLE 3
SEC analysis-oxidative stress

| | | Aggregation | Monomer | Small fragment |
|---|---|---|---|---|
| Antibody name | Condition(s) | % | % | (s) % |
| HFB10-1E1hG1 | Stored at 4° C. | 2.99 | 96.89 | 0.12 |
| | 0.1% H₂O₂, 0 h | 2.75 | 97.11 | 0.14 |
| | 0.1% H₂O₂, 6 h | 2.78 | 97.05 | 0.17 |
| | 1% H₂O₂, 0 h | 2.81 | 96.99 | 0.20 |
| | 1% H₂O₂, 6 h | 2.56 | 97.13 | 0.31 |
| | 0.1% t-BHP, 0 h | 3.02 | 96.81 | 0.18 |
| | 0.1% t-BHP, 6 h | 2.82 | 96.96 | 0.23 |

The SDS-PAGE analysis was shown in FIG. 5C. After an incubation in 0.1% H₂O₂, 1% H₂O₂, and 0.1% t-BHP (tert-butyl hydroperoxide) solution for 6 hours, no significant changes in HFB10-1E1hG1 were observed on the SDS-PAGE gel, except a slightly degradation on the non-reducing gel, indicating that the HFB10-1E1hG1 has good stability under such treatment conditions.

### Example 6.4 Freeze-Thaw Experiment of HFB10-1E1hG1

The antibody sample (HFB10-1E1hG1, 3.1 mg/mL, lot #CP181130004) was concentrated to 10 mg/mL (lot #JW20181203) through a centrifugal filtration device (Millipore, Cat #UFC503096). An appropriate amount of concentrated antibody sample was transferred to a clean 600 μl tube (3× tube), and the sample was frozen in liquid nitrogen for 2 minutes and thawed in a water bath at room temperature. The same procedure was repeated for 2 or 4 times or even more times.

The samples were analyzed by SEC-HPLC and SDS-PAGE as previously described. Note: 1) untreated samples stored at 4° C. were analyzed; 2) SEC running buffer was self-prepared with 100 mM NaH2PO4, 150 mM NaCl, pH 6.8.

For DSF-based thermal stability analysis: according to the manufacturer's instructions, 3 μg of each treated sample was used for each 25 μl reaction of the ProteoStat assay kit (Enzo Life Sciences, Cat #ENZ-51027-K400) in a PCR plate (Bio-Rad plate, Cat #HSP9655; Bio-Rad membrane, Cat #MSB1001). The heating program on the Bio-Rad PCR instrument (C1000 touch, CFX96 real-time system) was as follows: 25° C. for 2 minutes, increasing by 0.5° C. to 95° C. every 10 seconds. The fluorescence absorbance was read under the Texas Red mode. The Tm value was related to the lowest point of −dF/dT.

The SEC analysis was shown in Table 4. DSF analysis was described in Table 5. SEC analysis showed that no significant change in HFB10-1E1hG1 was observed on the SEC curve after freezing/thawing treatment for up to 5 cycles. DSF analysis showed that the Tm value of HFB10-1E1hG1 did not change significantly after the same treatment, indicating that HFB10-1E1hG1 has good stability under such treatment conditions.

TABLE 4
SEC freeze-thaw analysis

| | | Aggregation | Monomer | Small fragment |
|---|---|---|---|---|
| Antibody name | Condition(s) | % | % | (s) % |
| HFB10-1E1hG1 | Stored at 4° C. | 3.15 | 96.85 | 0 |
| | F/T cycle 1 | 3.03 | 96.97 | 0 |
| | F/T cycle 3 | 3.03 | 96.96 | 0 |
| | F/T cycle 5 | 2.95 | 97.05 | 0 |

TABLE 5
DSF freeze-thaw analysis

| Antibody name | Condition(s) | Tm (° C.) |
|---|---|---|
| HFB10-1E1hG1 | Stored at 4° C. | 74.5 |
| | F/T cycle 1 | 74.5 |
| | F/T cycle 3 | 74.25 |
| | F/T cycle 5 | 74.25 |

The SDS-PAGE analysis was shown in FIG. 5D. After freezing/thawing treatment for up to 5 cycles, no significant changes in HFB10-1E1hG1 were observed on the SDS-PAGE gel, indicating that HFB10-1E1hG1 has good stability under such treatment conditions.

### Example 7

It has been shown that the binding of agonistic antibodies with OX-40 can lead to down-regulation of receptor both in vitro and in clinical trials (Wang et al., Cancer Research 2019). It has been further hypothesized that the subsequent loss of target expression observed in patients after the first injection may limit the application of OX-40 agonistic antibodies in clinical trials (Wang et al., Cancer Research 2019). By using ex vivo activated naive T cells isolated from PBMCs, the present invention showed that treatment with HFB10-1E1hG1 resulted in less OX-40 reduction compared to traditional agonistic antibody treatments. The unique binding epitope and optimized binding kinetics minimized target degradation, thereby avoided the loss of target expression after the first injection, thus allowing continuous drug administration to the patients.

### Example 8

### Human PK Prediction of HFB10-1E1hG1

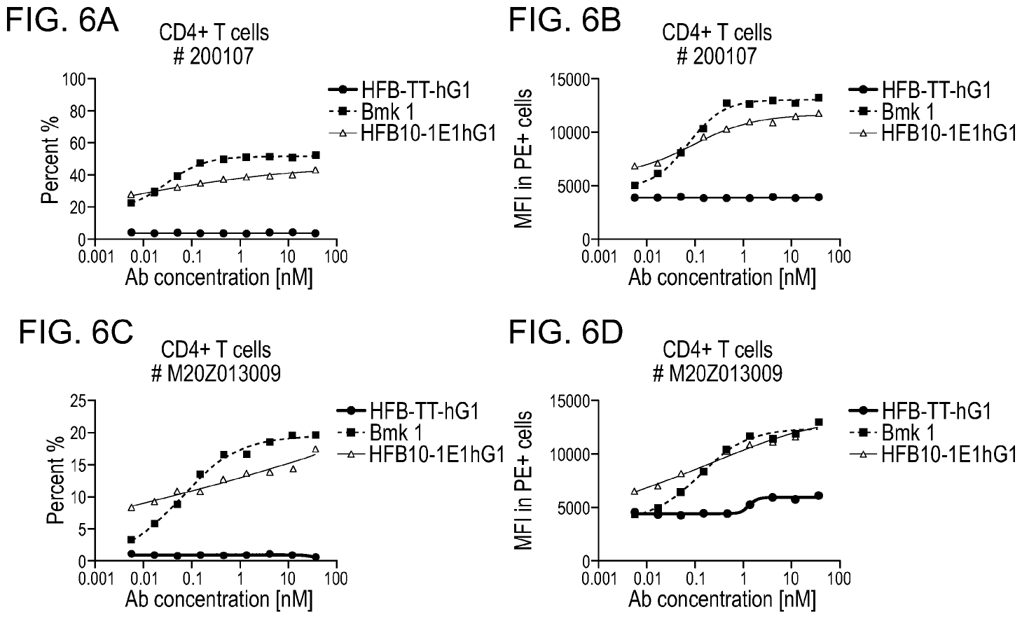

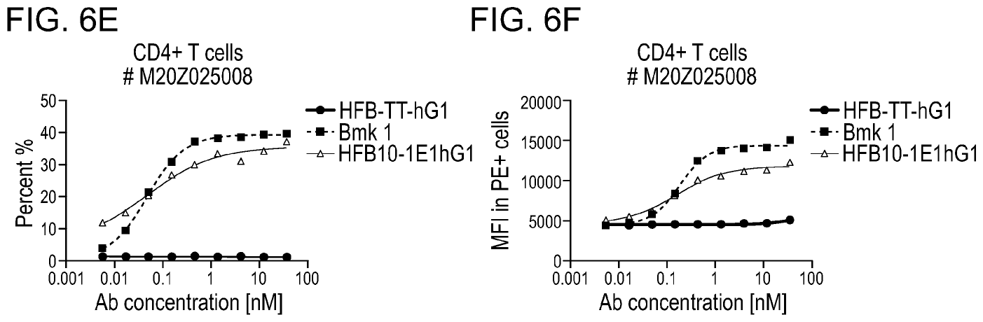

PK data in cynomolgus monkey was fitted into a 2-compartmental model. The fitted PK profile and observed data are shown in FIGS. 6A-6E The root mean square error of the fit is 0.17, and the R² is 0.99, indicating a good fit to the observed data.

The human 2-compartmental PK parameters were predicted based on Rule of Exponent (ROE) from corresponding monkey parameters (Dong et. al, 2011).

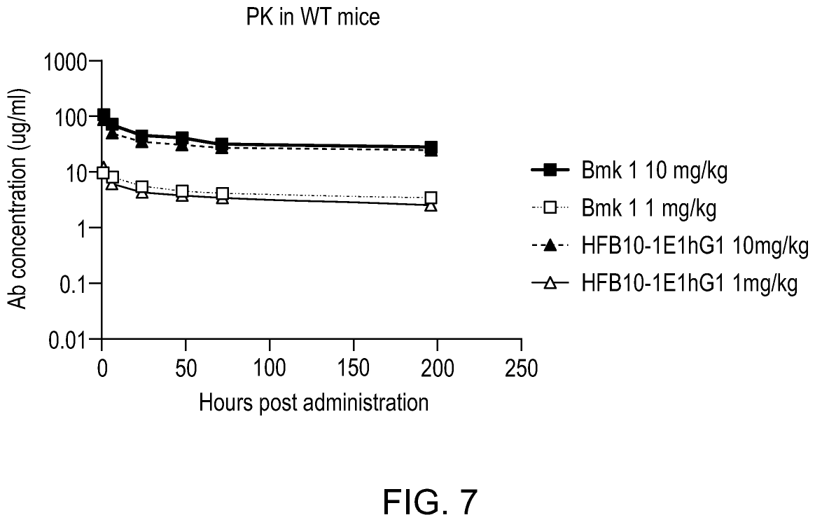

Human PK profile was then simulated using the predicted parameters. FIG. 7 shows an example of human PK profile at 1-hour post a single dose of intravenous infusion of HFB10-1E1hG1 at 1 mg/kg.

Minimum Human PAD Prediction Based on HFB10-1E1hG1 PK Data in hOX40 KI Mice

The Cₘₐₓ and AUC₍₀₋₇₂ₕ₎ values of 10 mg/kg HFB10-1E1hG1 in hOX40 KI mice were 160.5 μg/mL and 2591 μg/mL*h, respectively. Assuming linear PK, the Cₘₐₓ and AUC₍₀₋₇₂ₕ₎ values at 1 mg/kg HFB10-1E1hG1 would be 16 μg/mL and 259 μg/mL*h, respectively.

The predicted human Cₘₐₓ and AUC₍₀₋₇₂ₕ₎ values at 1 mg/kg HFB10-1E1hG1 are 23.7 μg/mL and 1213 μg/mL*h. Assuming linear PK in humans, the minimum PAD is predicted to be 0.68 mg/kg (based on Cₘₐₓ) or 0.21 mg/kg (based on AUC₍₀₋₇₂ₕ₎). To be conservative, human minimum PAD is assumed to be 0.21 mg/kg or a fixed dose of 15 mg (assuming standard body weight is 70 kg).

### Consideration of Dosing Frequency in Humans

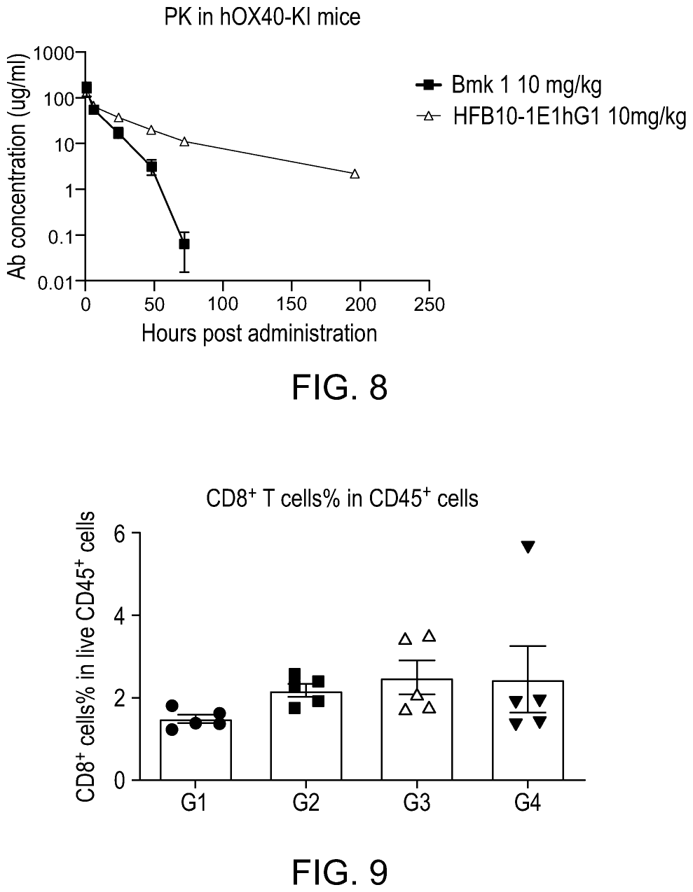

To maximize OX40 agonism and subsequent effector T cell expansion and regulatory T cell depletion in the tumor microenvironment, care should be taken not to saturate the OX40 receptor for an extended period of time. One strategy to ensure sufficient time in between OX40 agonism is to avoid significant accumulation of agonist of interest. We simulated the PK profile of HFB10-1E1hG1 at once every 2 weeks (Q2W), 3 weeks (Q3W) or 4 weeks (Q4W) schedule. The simulated PK profile at Q4W is shown in FIG. 8.

We then calculated the accumulation index of Cₘₐₓ and Cₘᵢₙ, and the Cₘₐₓ/Cₘᵢₙ ratio at steady state for each schedule. The results are listed in Table 2.

Based on the calculated AI and the Cmax/Cmin ratio, in addition to practical considerations, a 15 mg initial dose with Q4W schedule was selected for the first-in-human study of HFB10-1E1hG1.

Human PK of HFB10-1E1hG1 is predicted to be similar to a typical IgG1 monoclonal antibodies (mAbs) with a low clearance rate (CL, 0.084 mL/h/kg), low volume of distribution (Vdss, 0.084 L/kg), and a typical mAb half-life (T_{1/2}, 731 h, about 30 days).

The minimal human PAD of HFB10-1E1hG1 was predicted based on the adjusted human PK corresponding to cynomolgus monkey PK, the minimum efficacious dose from MC38 tumor efficacy studies in hOX40 KI mice, and HFB10-1E1hG1 exposure data in hOX40 KI mice. A minimal PAD of 0.21 mg/kg in humans was extrapolated based on the exposure and efficacy relationship in the KI mice. For convenience, an equivalent fixed dose of 15 mg is recommended as the starting dose in humans. The recommended dosing frequency is once every 4 weeks (Q4W) based on the predicted steady state PK profile at different dosing frequencies.

Here we evaluated the binding of HFB10-1E1hG1 to activated primary monkey T cells using flow cytometry.

CD4⁺ T cells serve as a positive control that binds to Bmk 1. HFB10-1E1hG1 was demonstrated to bind to the activated CD3/CD28 primary monkey T cells isolated from three different donors (Lot #200107, M20Z013009, and M20Z025008). The EC50 values calculated from the MFI dose response curves for each of the binding assays were 0.09 nM, 0.19 nM, and 0.17 nM, respectively.

The linear detection range of the ELISA assay was from 125 to 1000 μg/ml in experiment A and from 125 to 2000 μg/ml in experiment B. The LLOQ was 18.75 ng/ml in both experiments.

Here we evaluated the pharmacokinetics of Bmk 1 and HFB10-1E1hG1 in: 1) WT mice via i.v. administration with a single dose at 10 mg/kg and 1 mg/kg, and 2) hOX40-KI mice with a single dose at 10 mg/kg. The average concentration of Bmk 1 and HFB10-1E1hG1 over time was plotted on a semi-log plot as presented in FIGS. 7 and 8.

The systemic exposure of HFB10-1E1hG1 and Bmk 1 was achieved in all treated mice. In WT mice (FIG. 7), HFB10-1E1hG1 and Bmk 1 exhibited a similar dose-dependent linear clearance rate. The dose ratio of HFB10-1E1hG1 and Bmk 1 was 1:10. The ratios for Cmax and AUC(0-72h) of HFB10-1E1hG1 were 1:6.8 and 1:7.9, while those of Bmk 1 were 1:11.8 and 1:8.6, respectively.

The serum clearance rates of HFB10-1E1hG1 at 1 mg/kg and 10 mg/kg were 0.71±0.34 and 1.06±0.77 ml/h/kg in the WT mice, while it was 3.09±0.27 ml/h/kg at 10 mg/kg in the hOX40-KI mice. Similar serum clearance rate difference was observed for Bmk 1 PK. The serum clearance rates of Bmk 1 at 1 mg/kg and 10 mg/kg were 0.45±0.13 and 0.62±0.10 ml/h/kg in the wildtype mice, while it was 7.24±2.24 ml/h/kg at 10 mg/kg in the hOX40-KI mice. Because hOX40 was only expressed in the hOX40-KI mice, the higher HFB10-1E1hG1 clearance rate in hOX40-KI mice is possibly due to the target-mediated drug disposition (TMDD).

The steady state volume of distribution (Vdss) values of HFB10-1E1hG1 at 1 mg/kg and 10 mg/kg were 0.21±0.03 and 0.26±0.02 L/kg in the WT mice, while it was 0.14±0.01 L/kg at 10 mg/kg in hOX40-KI mice. Similarly, the Vdss values of Bmk1 at 1 mg/kg and 10 mg/kg were 0.19±0.02 and 0.21±0.01 L/kg in the WT mice, and it was 0.08±0.03 L/kg at 10 mg/kg in hOX40-KI mice.

Clear differences in terminal half-life (T_{1/2}) were also observed between WT and hOX40 KI mice for both antibodies. In the WT mice, the T_{1/2}s of HFB10-1E1hG1 at 10 mg/kg and 1 mg/kg were 239.72±140.48 h and 274.52±193.12 h, and those of Bmk1 at 10 mg/kg and 1 mg/kg were 252.04±51.82 h and 321.05±75.89 h, respectively. However, in hOX40 KI mice, the T₁/2 values at 10 mg/kg were considerably shorter, which were 38.65±4.84 h for HFB10-1E1hG1 and 5.45±1.09 h for Bmk 1.

### Example 9 Pharmacodynamic Study

Methods: 30 female human OX40 knock-in mice (C57BL/6 background) were inoculated subcutaneously at right flank with MC-38 tumor cells for tumor development. Nine days after tumor inoculation, 20 mice with tumor size ranging from 76-226 mm³ (average tumor size 155 mm³) were selected and randomly assigned into 4 groups based upon their tumor volumes. Each group had 5 mice. Each mouse received one of the four treatments from the day of randomization (defined as Day 0 (D0)). The four treatments were: 10 mg/kg Isotype control; 0.1 mg/kg HFB10-1E1hG1; 1 mg/kg HFB10-1E1hG1, and 10 mg/kg HFB1-1E1hG1. All treatments were administered via i.p. injections on D0, D3 and D7. The tumor sizes and animal body weights were measured at least three times per week. 6 hours post the third injection, tumor samples were collected for flow cytometry (FCM) analysis, blood samples were collected for receptor occupancy (RO) analysis, and plasma samples were sent to HiFiBiO for further analysis.

Results: The PD effect of HFB10-1E1hG1 was tested in the MC-38 tumor-bearing hOX40 KI mice. In both blood and tumor microenvironments (majorly in tumor), HFB10-1E1hG1 led to reduced OX40 expression in positive percentage and MFI on T cells, predominantly on CD4⁺ T cells. Meanwhile, HFB10-1E1hG1 also significantly decreased the Treg population. In the tumor microenvironment, the reduction of CD4⁺ T cells was primarily driven by a decrease of Tregs (CD25⁺FOXP3⁺ cells), and the proliferation of CD4⁺ T cells (Ki67⁺ cells) increased together with a reduction of CD69⁺ T cells. Meanwhile, the PD1 expression was reduced on the CD8⁺ T cells in the tumor microenvironment. All of these observations were HFB10-1E1hG1 dose-dependent in the treatment groups. Taken together, these data suggest that HFB10-1E1hG1 treatment activated the OX40 signaling pathway, which contributes to the durable immune responses observed in the tumor microenvironment for the efficacy study.

The effect of HFB10-1E1hG1 on OX40 expression in blood samples was analyzed using a mouse anti-hOX40 antibody (clone ACT35) that is non-competitive to HFB10-1E1hG1. The total OX40 expression on CD4⁺, CD4⁺CD25⁺, and CD11b⁺ monocytes was measured (data not shown). HFB10-1E1hG1 treatments led to significantly down-regulation of OX40 expression (both in positive percentage and MFI) on CD4⁺ and CD4⁺CD25⁺ T cells, including Tregs. No changes were observed for OX40 expression on the CD11b⁺ cells.

To evaluate target engagement upon HFB10-1E1hG1 treatments, anti-human IgG (hIgG) was used in combination with the non-competitive Ab ACT35 in FCM analysis. The percentage of hIgG⁺ population on the OX40⁺CD4⁺ T cells and on the OX40⁺CD4⁺CD25⁺ T cells was low. However, the MFI of hIgG⁺ on the OX40⁺CD4⁺CD25⁺ cells, including the Tregs has significantly increased in a HFB10-1E1hG1 dose-dependent manner.

On the OX40⁺CD11b⁺ cells, a dose-dependent high level of hIgG⁺ signal was observed in the isotype and the 10 mg/kg HFB10-1EhG1 treatment groups, suggesting that hIgG was captured by FcgRs on the CD11b⁺ cells.

Infiltrating immune cell populations in the tumor tissues were analyzed by flow cytometry (“FCM”). For all of the FCM analysis graphs exemplified here:
G1: Isotype control group; G2: 0.1 mg/kg HFB10-1E1hG1 treatment group; G3: 1 mg/kg HFB10-1E1hG1 treatment group; G4: 10 mg/kg HFB10-1E1hG1 treatment group
Each dot represents data from an individual animal, the rectangle bars represent group means, and error bars represent SEM.

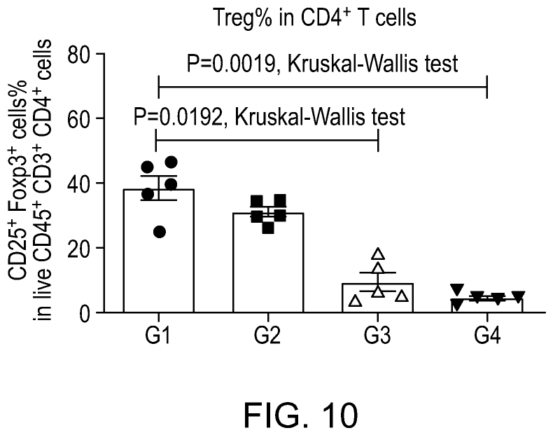

Percentages of T cells (CD3⁺), CD4⁺ T, CD8⁺ T cells and Tregs (CD3⁺CD4⁺CD25⁺Foxp3⁺) were measured, and the selected results are shown in FIGS. 9 and 10. Percentages of Naïve T cells (CD3⁺CD44⁻CD62L⁺), memory T cells (CD3⁺CD44⁺CD62L⁺) and effector T cells (CD3⁺CD44⁺CD62L⁻) were also measured (data not shown). As a result, the HFB10-1E1hG1 (G3 and G4, but not G2) treatment significantly decreased the percentage of CD4⁺ T cells, Treg, naïve T cells, and naïve CD4⁺ T cells compared to the isotype control (G1) treatment. Moreover, all of the HFB10-1E1hG1 treatments significantly decreased the percentage of memory T cells and memory CD4⁺ T cells compared to the control treatment. Although all HFB10-1E1hG1 treatment groups decreased the percentage of memory CD8⁺ T cells, but only the G2 and G3 treatments were statistically significant. No significant changes were observed on T cells (CD3⁺), CD8⁺ T cells and effector T cells populations among the treatment groups.

The effect of HFB10-1EhG1 treatments on the expression of OX40, activation and proliferation of different T cell subtypes in tumors were examined. The effect of HFB10-1E1hG1 on the total OX40 expression was analyzed using a mouse anti-hOX40 antibody (clone ACT35) that is non-competitive to HFB10-1E1hG1. OX40 expression on CD3⁺, CD4⁺, CD8⁺ T cells, and Tregs (CD4⁺CD25⁺FoxP3⁺) was measured (data not shown). Compared to the isotype control group (G1), HFB10-1E1hG1 treatment significantly decreased OX40 expression on the CD3⁺ and CD4^{CD} T cells both in percentage and in MFI (positive percentage of OX40 expression on the left panels and MFI of OX40 on the right panels). Also, HFB10-1E1hG1 treatments significantly decreased OX40 expression in MFI but not in percentage on Tregs (data not shown). Moreover, HFB10-1E1hG1 treatment significantly decreased OX40 expression in percentage but not in MFI on the CD8⁺ T cells.

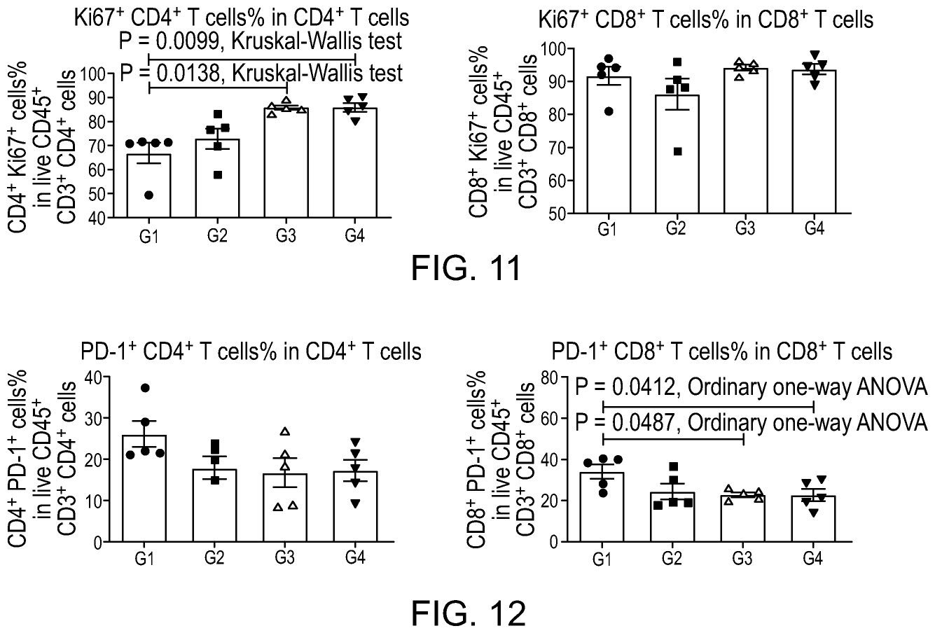

Compared to the isotype control group (G1), treatments of HFB10-1E1hG1 (G3 and G4) significantly decreased the percentage of CD69⁺CD4⁺ T cells and PD-1⁺CD8⁺ T cells (FIG. 12) and increased the percentage of proliferating (Ki67⁺) CD4⁺ T cells (FIG. 11).

The observed results above are HFB10-1E1hG1 dose-dependent.

### Example 10 Epitope Mapping

HFB301001 was developed as an agonistic monoclonal anti-OX40 antibody. Table 1 shows the CDR, VH/VL and HC/LC sequences of HFB301001.

The binding epitopes were characterized by epitope binning using a competitive ELISA assay to explore the OX40 binding epitopes of different anti-OX40 antibodies relative to the previously published antibodies that have been selected and OX40 ligand binding epitopes. In short, a plate was coated with 50 μl 1 μg/ml anti-OX40 capture antibody, OX40L, or isotype control, and incubated at 4° C. overnight. Then, the plate was blocked with 1% BSA in PBST at room temperature for 2 hours. Next, 50 μl/well of Biotin-OX40 (Lot #160921111, ChemPartner; Tokyo, JP) was added. Biotin-OX40 was used at concentrations diluted 1 to 3 times for an 8-point dilution from 0.5 μg/mL, and incubated at 37° C. for 1 hour. HRP-conjugated streptavidin (1/5000 dilution; 50 ul/well) was added and incubated at 37° C. for 1 hour, then 100 μl/well of TMB was added and incubated at room temperature for 15 min. The reaction was stopped by adding 50 μl/well of ELISA stop solution. The OD value was determined with a Thermo Multiscan sky spectrophotometer at a wavelength of 450 nm.

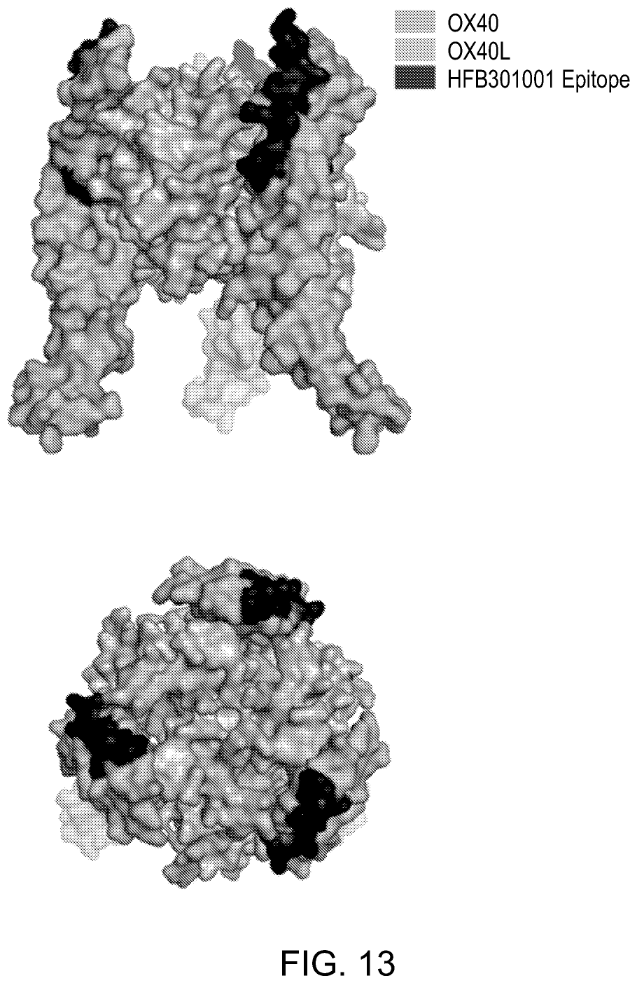

A more detailed resolution of the bound epitope was obtained by using hydrogen deuterium exchange mass spectrometry. In short, mass spectrometry (HDX-MS) was used for the H/D exchange epitope mapping to determine the amino acid residues of OX40 (recombinant human OX40) that interact with HFB301001. A general description of the H/D exchange method is set forth in, for example, Ehring (1999) Analytical Biochemistry 267(2):252-259; and Engen and Smith (2001) Anal. Chem. 73:256A-265A. The His-tagged OX40 antigen protein was purchased from Sinobiological (catalog number 10481-H08H; Sinobiological, Inc.; Beijing, CN). The HDX-MS experiment was performed for peptide mass measurement on the integrated HDX/MS platform provided by Novabioassays LLC and Thermo Q Exactive HF mass spectrometer. 4.5 μL of all samples was incubated with 75 μL of deuterium oxide labeling buffer (1×PBS, pD 7.4) at 20° C. for 300 seconds, 600 seconds, 1800 seconds, and 3600 seconds. The hydrogen/deuterium exchange was quenched by adding 80 μL of 4 M guanidine hydrochloride, 0.85 M TCEP buffer (final pH value of 2.5). Subsequently, the quenched samples were subjected to on-column pepsin/protease XIII digestion, followed by the LC-MS analysis. The mass spectrum data were recorded in MS mode only. Pepsin/Protease XIII digestion, LC-MS and data analysis. After HDX labeling, the samples were denatured by adding 80 μL of 4 M guanidine hydrochloride, 0.85 M TCEP buffer (final pH 2.5) and incubating at 20° C. for 3 min. Then, the mixture was subjected to on-column pepsin/protease XIII digestion using the internal packed pepsin/protease XIII (w/w, 1:3) column. The resulting peptides were analyzed using a UPLC-MS system consisting of a Waters Acquity UPLC connected to a Q Exactive™ plus Hybrid Quadrupole-Orbitrap mass spectrometer (Thermo; Waltham, MA). The peptides were separated on a 50×1 mm C8 column with a 20.5 min gradient starting from 2% to 33% solvent B (acetonitrile with 0.2% formic acid). Solvent A was 0.1% formic acid in water. The injection valve, enzyme column and related connecting pipes were located in a cooling box kept at 15° C. The second on-off valve, C8 column and related connecting stainless steel pipe fittings were located in the refrigerated recycling box kept at −6° C. Peptide identification was performed by searching the MS/MS data of HFB301001 sequence using the modified Byonics (Protein Metrics; San Carlos, CA) software that takes the non-specific enzyme digestion and human glycosylation as common variables. The mass tolerances of precursor ions and product ions were 10 ppm and 0.02 Da, respectively. HDX WorkBench software (version 3.3) was used to process the raw MS data in order to analyze the H/D exchange MS data (J. Am. Soc. Mass Spectrom. 2012, 23 (9), 1512-1521). The average mass difference between the deuterated peptide and its unlabeled form (t0) was used to calculate the deuterium absorption level. The deuterium absorption levels of the same peptide in a single antigen sample and an antigen-antibody complex sample were compared. Relative differences greater than 5% were considered significant. The epitope regions were defined as the multiple overlapping peptides with significant deuterium absorption. A total of 46 peptides from hOX40-his, representing 70% sequence coverage of hOX40, were identified from hOX40.his alone and hOX40-his-HFB301001 complex (FIG. 13). Any peptide that exhibited a percentage difference in D absorbance greater than 5% was defined as being significantly protected. For hOX40-his, the polypeptide in the region corresponding to amino acids 56-74 CSRSQNTVCRPCGPGFYND was considered as an epitope for being significantly protected by HFB301001. As shown in FIG. 13, the epitope region was mapped to the 3D model of the OX40/OX40L complex, which was adapted from Compaan and Hymowitz. Structure. 2006, 14 (8), 1321-30. The epitope of HFB301001 was defined as amino acids 56-74 of SEQ ID NO: 33, CSRSQNTVCRPCGPGFYND (SEQ ID NO: 34).

### Example 11 Agonistic Activity of OX40 Ligand

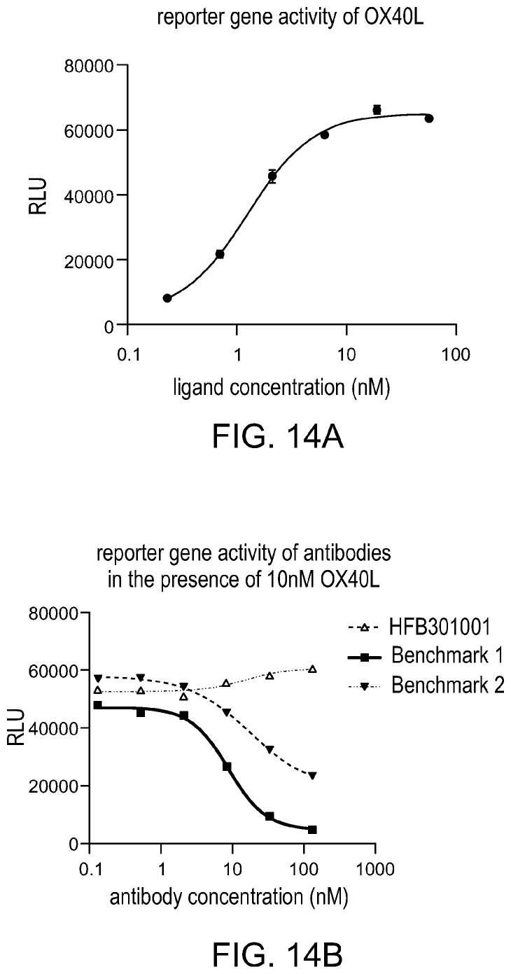

To evaluate the agonistic activity of the OX40 ligand (OX40L), the OX40 bioassay kit (Promega, catalog number CS197704; Promega; Madison, WI) was used according to the manufacturer's instruction. The assay used a genetically engineered Jurkat T cell line that expresses human OX40 (OX40 effector cells) and a luciferase reporter gene driven by a response element. In short, OX40 effector cells were cultured in the assay buffer the day before use. On the day of the assay, a serial dilution of OX40L was added to the plate. After 5 hours of induction, the bioluminescence signal was quantified using the Bio-Glo Luciferase Assay System. The results showed that OX40L induced the bioluminescence signal of OX40 effector cells in a dose-dependent manner (FIG. 14A).

To study the effect of anti-OX40 antibody on the OX40-L agonistic activity, OX40 effector cells were incubated with a serially diluted anti-OX40 antibody (HFB301001, Benchmark 1 or Benchmark 2) in the presence of 10 nM OX40L. Benchmark 1 and Benchmark 2 are previously published anti-OX40 antibodies and were selected because the antibody epitope is furthest away from the cell membrane on OX40, while the other benchmarks are described as binding near the ligand binding pocket of OX40. The bioluminescence signal was quantified using the Bio-Glo Luciferase Assay System 5 hours after the induction. Both Benchmark 1 and Benchmark 2, but not HFB301001, blocked the agonistic effect of OX40L in a dose-dependent manner (FIG. 14B). The results showed that HFB301001 recognizes different binding sites on OX40 as compared to Benchmark 1 and Benchmark 2, and the binding sites do not interfere with the agonistic function of OX40L.

### Example 12 OX40 Receptor Modulation

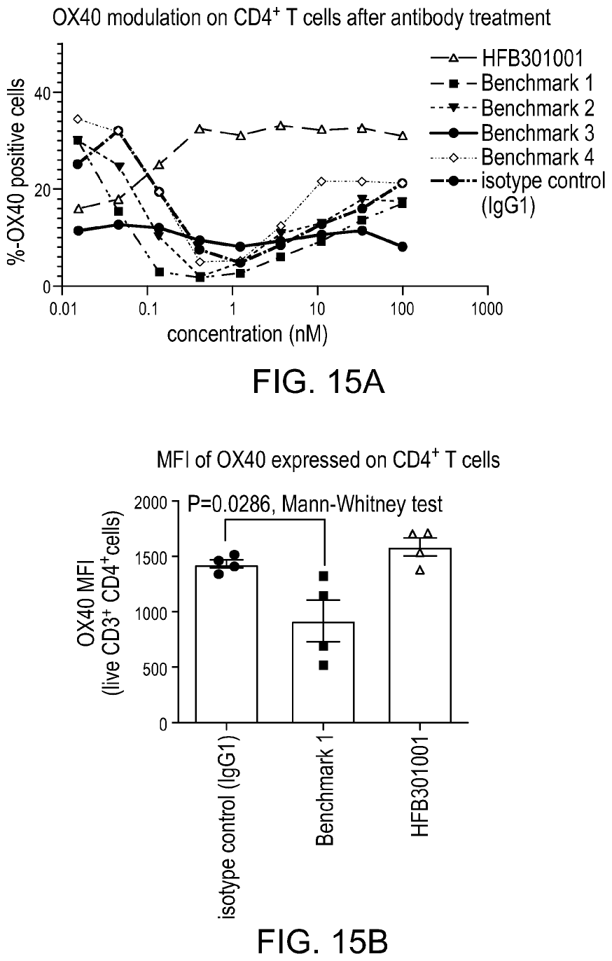

To study the regulatory effect of the binding of anti-OX40 antibody to OX40 receptor, CD4⁺ T cells from human peripheral blood mononuclear cells (PBMC) was isolated using the Miltenyi's Human CD4⁺ T Isolation Kit (Catalogue 130-096-533; Miltenyi Biotec; Bergisch Gladbach, Germany). The primary CD4⁺ T cells were incubated with the serially diluted Benchmark antibodies or HFB301001, and were simultaneously activated by the 0.5 μg/mL plate-bound anti-CD3 antibody (clone OKT3, eBioscience, catalog 16-0037-81) and 2 μg/mL soluble anti-CD28 antibodies (clone CD28.2, eBioscience, catalog 16-0289-81; eBioscience, Inc.; San Diego, CA). After 3 days of incubation, the expression of total surface OX40 was detected by staining with the anti-OX40 antibody (PerCP-Cy5.5, clone ACT35, Thermo Fisher, catalog 17-1347-42). Compared with the isotype control, HFB301001 enhanced stable OX40 expression on the cell surface (FIG. 15A). In all benchmark treatment groups, the surface expression of OX40 exhibited a U-shape as the increase of antibody concentration: the level of OX40 decreased initially, reached its lowest point at about 1 nM, and then increased again.

### Example 13 Binding Affinity Evaluation

A biofilm layer interference (BLI) experiment was performed on the Octet QKe instrument to determine the binding affinity of the recombinant dimer (mFc-tagged) formed between HFB301001 and the human OX40 protein. Various concentrations of recombinant human OX40 protein were mixed with the sensor-captured test antibodies and the isotype control at pH 7.4 and 25° C., following a dissociation phase. The changes of binding signal were recorded, and the kinetic analysis of binding parameters were determined using the Model: 1:1 with the mass transfer limitation. The running buffer solution was prepared with PBS, pH 7.4, containing 0.1% BSA and 0.1% Tween 20. Prior to the kinetic binding experiment, the anti-human Fe capture sensor was rinsed with the running buffer, and the instrument was prewarmed to 25° C. The tested antibody and isotype control antibody were diluted to 200 nM with the running buffer. The OX40-mFc protein was diluted to 250, 125, 62.5, 31.25, and 15.125 nM with the running buffer. The binding of OX40 protein and its antibodies was performed in duplicate with the running buffer at pH 7.4 and 25° C. In short, first, the anti-human Fc capture sensor was immersed in the running buffer for 300 seconds to set the first baseline. The sensor was then mixed with 200 nM test antibody solution for 600 seconds to load the antibody. The sensor was again immersed in the running buffer for 300 seconds to set the second baseline, and then associated with various concentrations of OX40 antigen for 900 seconds. Then dissociation was performed by immersing the sensor in the running buffer for another 900 seconds. In parallel, one sensor was selected as a reference, in which all loading, association, and dissociation steps were performed in the running buffer. Another sensor was selected as a negative control experiment, in which the HFB-TT-hG1 isotype control antibody was used for the loading step and 500 nM OX40-mFc protein was used for the association step. The sensorgrams were analyzed by Fortebio data analysis software version 8.2 (ForteBio; Fremont, CA). First, the signals were subtracted from the negative control experiment, and compared to the baselines. The kinetic parameters were obtained by entirely fitting these specific sensorgrams with different antigen concentrations to the binding Model: 1:1. The equilibrium dissociation constant (KD) was calculated based on the ratio of the dissociation rate constant to the association rate constant (KD=kd/ka). The kinetic binding parameter of the interaction between HFB301001 and human OX40 protein was determined using BLI technology. The assay measures the interaction between OX40 protein in certain range of concentration and the captured HFB10-1E1 and other antibodies at 25° C. and pH 7.4. The binding affinity table below summarizes the calculated kinetic binding parameters. HFB301001 has a high affinity for the recombinant human OX40 (hOX40.mFc) with a K_{D} value of 4.5 nM at 25° C. and pH 7.4. The other two antibodies showed a slower dissociation rate, resulting in a smaller K_{D} value, 0.49 nM for Benchmark 1 and 0.58 nM for Benchmark 2. The kinetic binding parameter of the interaction between HFB301001 and human OX40-mFc was determined by BLI technology at 25° C. and pH 7.4. HFB301001 specifically bound to recombinant human OX40-mFc protein with much greater affinity and faster dissociation rate compared to other tested antibodies.

Binding affinity table

| | kinetic binding parameters | | | | | |
|---|---|---|---|---|---|---|
| | Experiment A | | | Experiment B | | |
| Antibody | kₐ (1/Ms) | k_{d} (1/s) | K_{D} (M) | kₐ (1/Ms) | k_{d} (1/s) | K_{D} (M) |
| HFB301001 | 9.7E+04 | 4.3E−04 | 4.5E−09 | 9.7E+04 | 4.3E−04 | 4.4E−09 |
| Benchmark1 | 1.5E+05 | 7.4E−05 | 4.9E−10 | 9.8E+04 | 5.9E−05 | 6.0E−10 |
| Benchmark2 | 1.1E+05 | 6.1E−05 | 5.8E−10 | 1.5E+05 | 7.6E−05 | 5.1E−10 |

### Example 14 In Vivo Tumor Model

The MC-38 murine colon cancer model was used for human OX40 (hOX40) knock-in (KI) mice (catalog NM-HU-00041, Shanghai Model Organisms Center, Inc.). To evaluate the pharmacodynamic (PD) effect of HFB301001 in the MC-38 murine colorectal cancer model, 45 female hOX40 KI mice were subcutaneously inoculated with MC-38 tumor cells (8×10⁵ cells per mouse) on the right side for tumor development. Eight days after tumor inoculation, 12 mice with a tumor size between 104-243 mm³ (average tumor size of 181 mm³) were selected and randomly divided into 3 groups with 4 mice in each group based on their tumor volume. Each treatment group received 10 mg/kg of either anti-OX40 antibody HFB301001 or anti-OX40 antibody Benchmark 1, or the IgG1 isotype control injection. All treatments were administered intraperitoneally (i.p) on the day of random grouping (DO).

OX40 expression on the CD4⁺ T cells was measured by flow cytometry 24 hours after the third injection with 10 mg/kg anti-OX40 antibody. Benchmark 1, but not the HFB301001, induced a significant down-regulation of OX40 expression on CD4⁺ T cells in the blood (FIG. 15B).

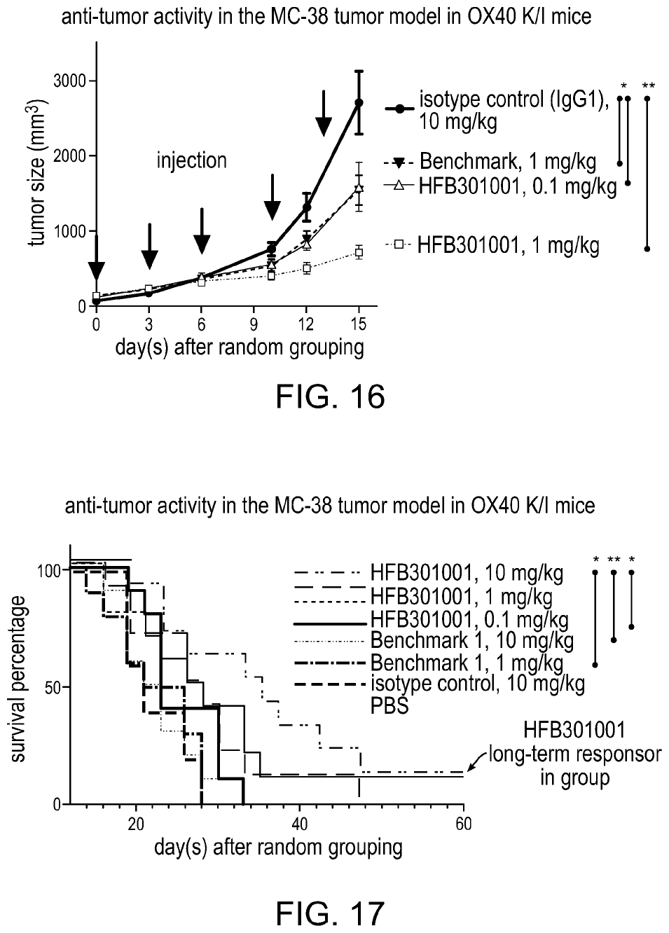

To evaluate the in vivo anti-tumor activity of HFB301001 in the MC-38 murine colorectal cancer model, 26 female hOX40 KI mice were subcutaneously inoculated with MC-38 tumor cells (8×10⁵ cells per mouse) on the right side for tumor development. Seven days after tumor inoculation, 20 mice with tumor size between 101-175 mm³ (average tumor size of 133 mm³) were selected and randomly divided into 4 treatment groups, with 5 mice in each group, according to their tumor volume. The four treatment groups were: anti-OX40 antibody HFB301001 at doses of 1 mg/kg and 0.1 mg/kg, anti-OX40 antibody Benchmark 1 at a dose of 1 mg/kg, and IgG1 isotype control at a dose of 10 mg/kg. All treatments were administered intraperitoneally on D0, D3, D6, D10, and D13. The arrow represents the treatment time point, the error bar represents the standard error of the mean, and the significance level was calculated by one-way ANOVA at the last time point (*: p value<0.05, **: p value<0.01). The tumor size represented by tumor diameter (width and length) was measured by digital calipers on D0, D3, D6, D10, D12 and D15 after random grouping. FIG. 16 suggested that HFB301001 significantly inhibits tumor growth compared to the control and has greater anti-tumor growth effect than Benchmark 1.

In order to evaluate the in vivo anti-tumor activity of HFB301001 in the MC-38 murine colorectal cancer model, 80 hOX40 KI mice were subcutaneously inoculated with MC-38 tumor cells (8×10⁵ cells per mouse) on the right side for tumor development. Seven days after tumor inoculation, 65 mice with tumor size between 42-147 mm³ (average tumor size of 82 mm³) were selected and randomly divided into 7 treatment groups according to their tumor volume, with 10 mice in each group (excepting that there were only 5 mice in the PBS group). The treatment started on the day of random grouping (defined as day 0 (DO)). The seven treatment groups were: anti-OX40 antibody HFB301001 at doses of 10 mg/kg, 1 mg/kg and 0.1 mg/kg, anti-OX40 antibody Benchmark 1 at doses of 10 mg/kg and 1 mg/kg, IgG1 isotype control at dose of 10 mg/kg, and PBS. All treatments were administered intraperitoneally on D0, D3, D6, D10, and D13. From the beginning of treatment to Day 61, the tumor size and animal body weight were measured at least three times a week. Significance level was calculated by log-rank test (*: p value<0.05, **: p value<0.01). The survival rate was monitored for 60 days after random grouping. The survival rate for mice treated with 10 mg/kg HFB301001 was significantly higher than that of mice treated with 10 mg/kg Benchmark 1 (FIG. 17).

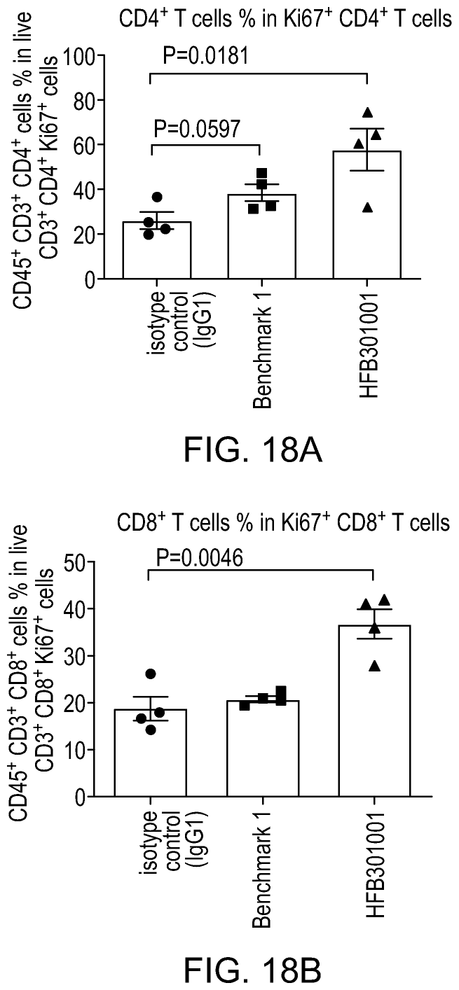

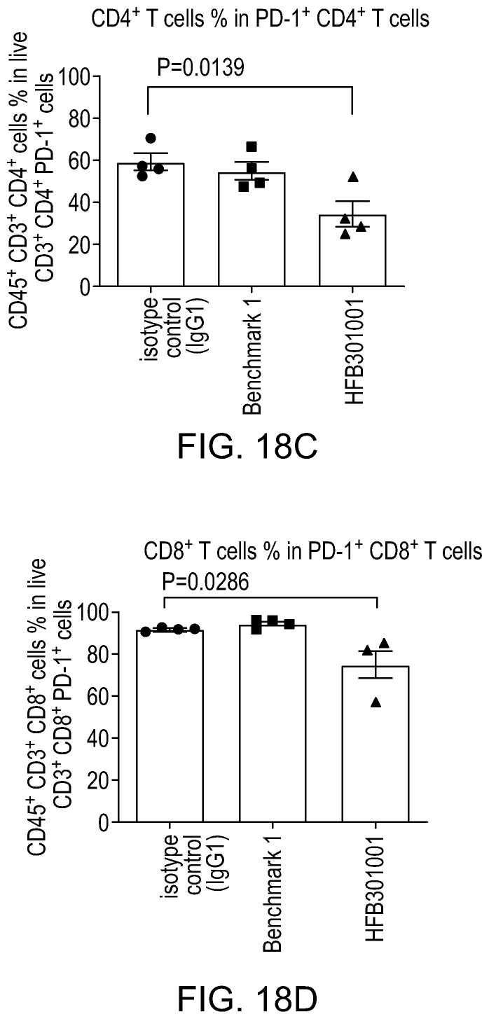

To further study the efficacy of anti-OX40 antibody HFB301001, changes induced by tumor T cells at 24 hours after the third treatment with anti-OX40 antibody were measured by flow cytometry. Eight days after tumor inoculation, 12 mice with a tumor size between 104-243 mm³ (average tumor size of 181 mm³) were selected and randomly divided into 3 groups, with 4 mice in each group, according to their tumor volume. The HFB301001 treatment has significantly increased KI67⁺ cells (FIG. 18A to FIG. 18B) and decreased PD-1⁺ cells in both CD4⁺ and CD8⁺ T cells (FIG. 18C to FIG. 18D). In addition, both the benchmark and HFB301001 treatments resulted in a significant decrease in tumor Treg, wherein the reduction in the HFB301001 group was more pronounced (FIG. 18E).

### Example 15 HFB301001 Effectively Induces OX40 Signaling

In reporter cell based experiments using engineered Jurkat T cells (also known as NF-kB-Luc2/OX40 reporter Jurkat cells, in which the Luc2 reporter is under the transcriptional control of a promoter activated by the NF-kB pathway upon OX40 activation, see Example 3.1) as OX40 effector cells, and FcγRIIb expressing CHO-K1 cells to provide antibody cross-linking, HFB301001 (HFB10-1E1hG1) showed a consistent dose-dependent agonistic activity.

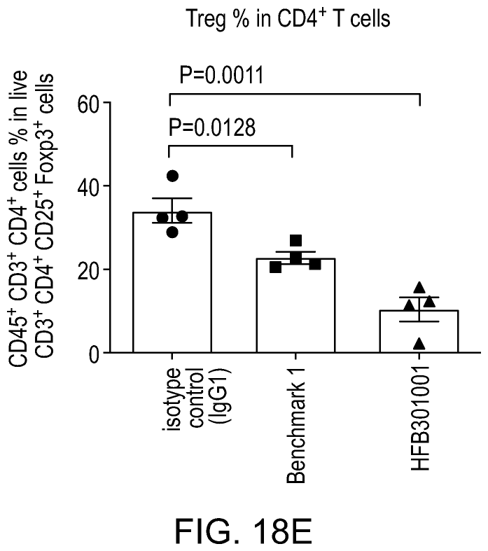

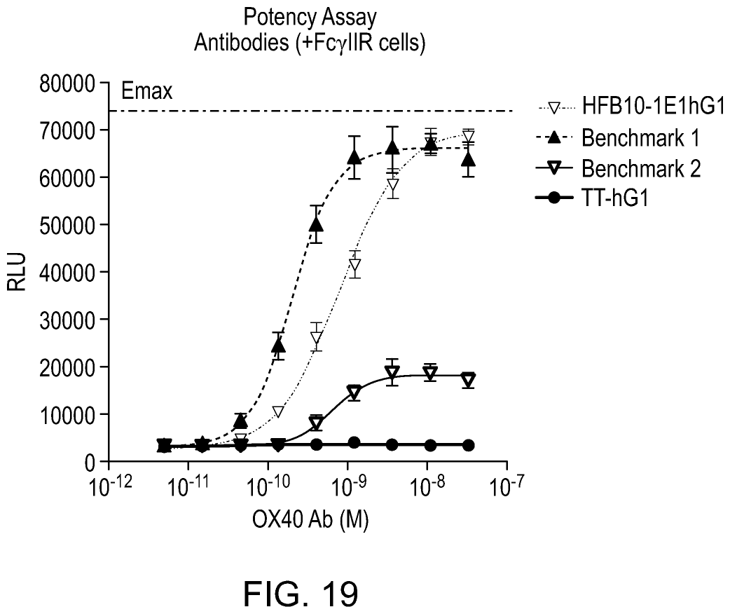

HFB301001 agonistic activity was less potent than Benchmark 1 based on EC50 values but had similar Eₘₐₓ values (see FIG. 19). The experiment was repeated 2 more times with similar results. The data showed HFB301001 effectively induced OX40 signaling.

### Example 16 Effects of HFB301001 on OX40 Signaling in Response to OX40 Ligand

OX40 reporter cells (NF-kB-Luc2/OX40 reporter Jurkat cells) in the absence of FcγRIIb expressing cells were used to study if OX40 antibodies block OX40L binding and signaling in OX40⁺ cells. Briefly, OX40 reporter cells were incubated with a range of concentrations of antibody and OX40L, and luciferase signal was a readout for OX40 signaling.

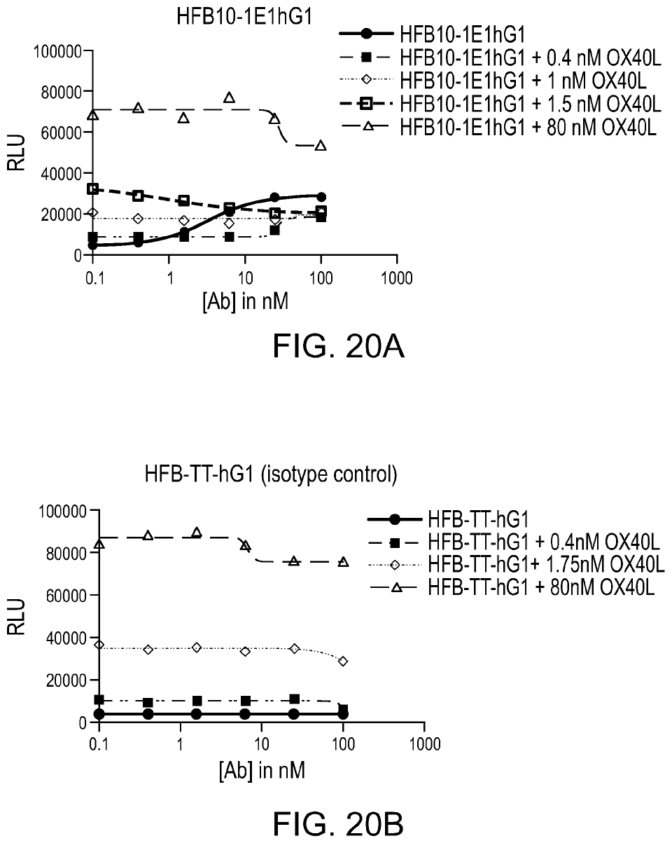

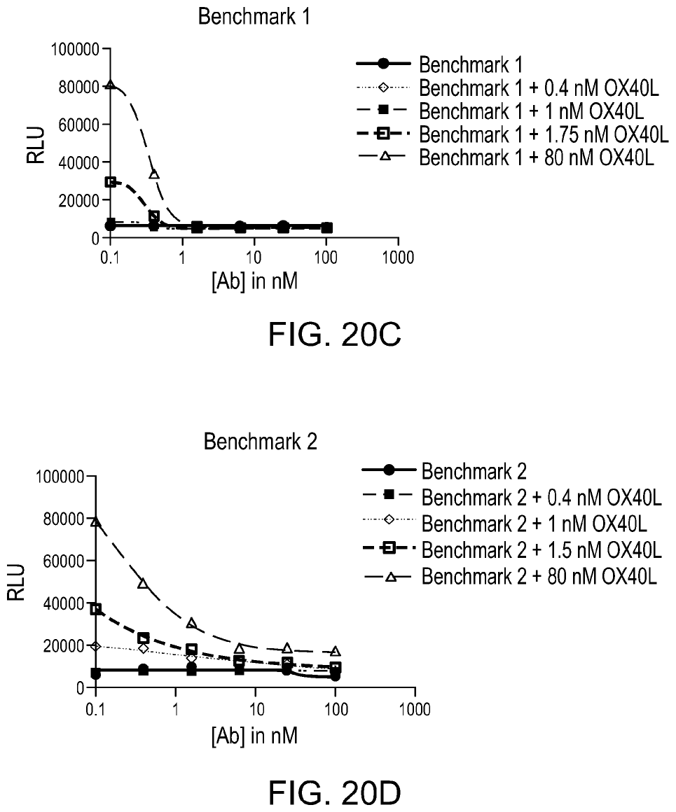

Benchmark 1 and Benchmark 2 antibodies showed a dose-dependent inhibitory effect on OX40L-induced OX40 signaling at different OX40L concentrations (see FIGS. 20C and 20D). However, HFB301001 (HFB10-1E1hG1) displayed minimal or no inhibition of signaling induced by OX40L in the OX40 reporter cells, especially at 80 nM concentration of OX40L (FIG. 20A).

This experiment suggests that HFB301001 does not interfere with endogenous OX40L signaling. These data are consistent with the binding epitope data. As endogenous OX40L signaling may play an essential role in modulation of immune network, keeping it intact may represent an advantage for HFB301001 compared to its ligand-blocking counterparts.

### Example 17 Binding of HFB301001 to Human CD4⁺CD3⁺ T Cells

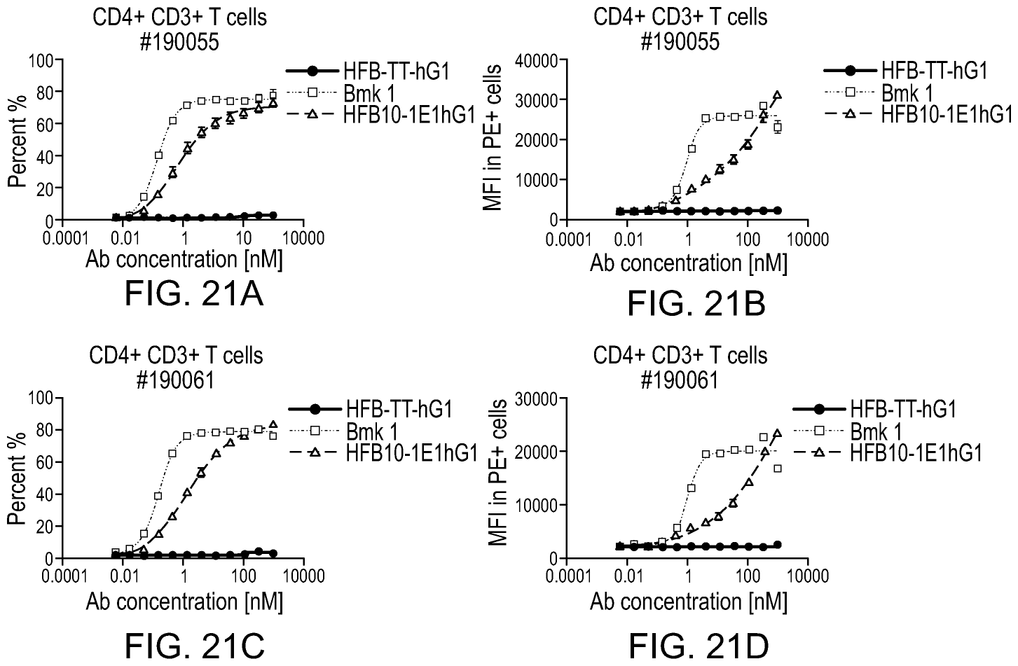

Activation of human T cells were evaluated using CD3⁺ T cells isolated from two human donors' peripheral blood mononuclear cells (hPBMCs) pre-activated using CD3/CD28 Dynabeads. CD3/CD28-induced OX40 was predominantly expressed on CD3⁺CD4⁺ T cells. HFB301001 bound to activated primary human CD4⁺CD3⁺ T cells from each donor with EC50 values of 0.65 and 1.36 nM, respectively (see FIGS. 21B and 21D).

HFB301001 exhibited similar binding to primary CD4⁺ T cells isolated from monkey PBMCs pre-activated using CD3/CD28 antibodies with binding EC50 values calculated from the MFI dose response curves from three independent experiments ranging from 0.09 to 0.19 nM (data not shown).

These data demonstrates that HFB301001 binds to activated human or monkey CD4⁺ T cells. The fact that the binding affinity is higher for monkey T cell suggests that monkey toxicology studies are unlikely to under-estimate potential safety issues in human.

### Example 18 OX40 Expression in Primary CD4⁺ T Cells Following Treatment with HFB301001

The effects of OX40 monoclonal antibodies on activated human T cells were examined. T cells were isolated from two different donors, #191223-B and #190061. OX40 expression on T cells was determined by staining with anti-OX40 antibody (clone ACT35).

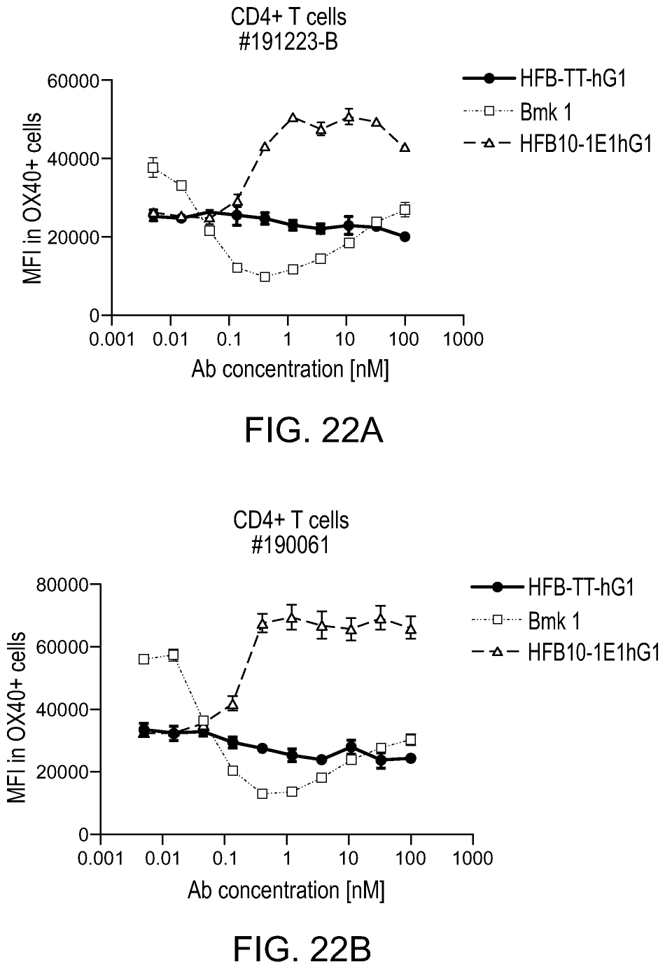

HFB301001 increased total surface levels of OX40 expressed on CD3/CD28-stimulated peripheral human CD4⁺ T cells in a dose-dependent manner and reached a plateau at around 1 nM concentration. In contrast, other anti-OX40 antibodies (all in clinical development; represented by Bmk 1) induced a decrease of surface OX40 levels on these CD4⁺ T cells (see FIGS. 22A and 22B).

The reduction of OX40 levels on T cells by OX40 antibodies likely diminishes the potential for OX40 agonistic antibodies to reach optimal clinical benefit. Given that this effect is not observed for HFB301001, a better clinical efficacy might be expected.

### Example 19 Effects of OX40 Antibody on T Cell Functions

The effects of OX40 monoclonal antibodies on human T cell functions were studied in co-stimulatory activity experiments in vitro where anti-OX40 antibodies were tested in the presence of superantigen Staphylococcus aureus enterotoxin A (SEA). Human IL-2 was quantified in culture supernatants after 70 hrs of stimulation.

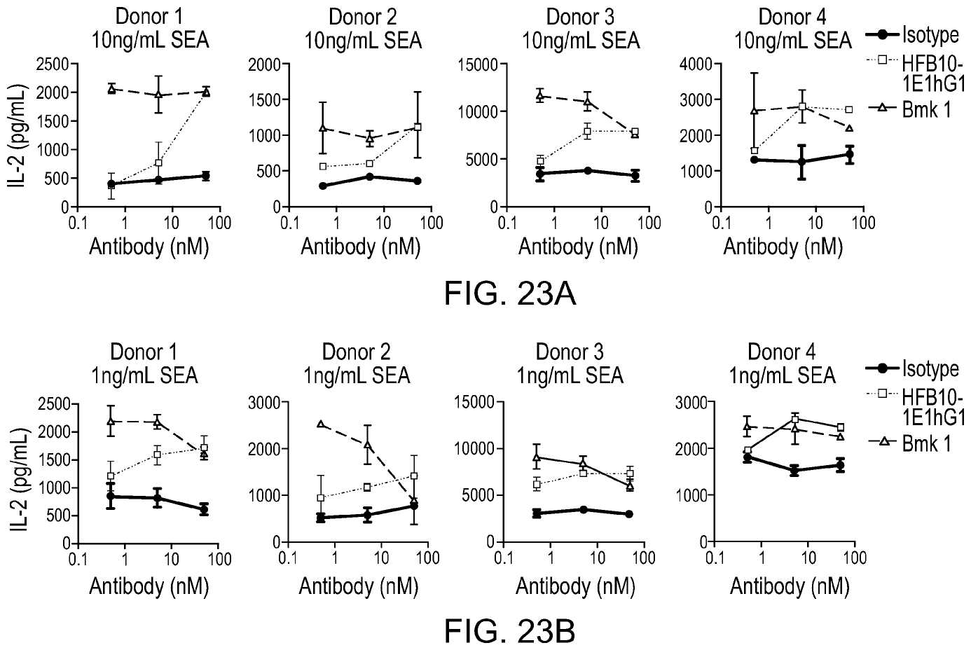

No clear dose-dependency was observed with Benchmark 1 (Bmk 1) antibody, especially when 1 ng/mL (SEA) was used (FIG. 23B). At the lower SEA level, lower level of IL-2 production was produced at higher concentration of Bmk 1 antibody in all 4 donor PBMCs.

In contrast, HFB301001 (0.5-50 nM) increased cytokine IL-2 levels in human PBMCs stimulated by the SEA (at 1 or 10 ng/mL) in a dose-dependent manner (FIGS. 23A and 23B). The experiment was repeated once with PBMCs from another 4 donors with similar results (data not shown).

While not wishing to be bound by any particular theory, the better dose dependency of HFB301001 may be, at least partially, due to the lack of receptor down-regulation and no blocking of endogenous ligand.

The data demonstrated that HFB301001 augmented T cell activation with dose dependency, and suggested that HFB301001 may be more likely to achieve optimal efficacy in clinical setting compared to other OX40 monoclonal antibodies in clinical development.

### Example 20 Effects of HFB301001 on Antibody-Dependent Cellular Cytotoxicity (ADCC)

As depletion of T-reg is one of the potential mechanisms for anti-tumor efficacy by OX40 agonist antibodies, CD16-NF-AT-Luc reporter Jurkat cells were used in the presence of target cells (hOX40-transfected Expi293F cells) and OX40 monoclonal antibodies to study the antibody-dependent cellular cytotoxicity (ADCC) potential of these antibodies.

HFB301001 dose-dependently induced CD16 signaling (which represent potential ADCC activity) in effector cells in the presence of hOX40 expressing target cells. EC50 values of 1.2, 0.7, 0.5, and 0.3 nM were resulted at effector to target cell (E:T) ratios of 1:1, 3:1, 6:1, and 12:1, respectively (Table 1).

Bmk 1 also exhibited ADCC activity in a dose-dependent manner with EC50 values of 2, 0.8 and 0.5 nM at E:T ratios of 1:1, 3:1 and 6:1, respectively; Bmk 2, being IgG2 isotype, did not show detectable ADCC activity as expected (Table 1).

In repeated experiments, potential ADCC activity induced by OX40 antibodies in CD16-NF-AT-Luc reporter Jurkat cells was tested with the E:T ratios of 3:1 and 6:1 with similar results (data not shown).

These data indicated HFB301001 maintained ADCC effector function.

TABLE 1
EC₅₀ Values of Anti-OX40 mAbs at Different E:T Ratios

EC₅₀ (nM)

| mAbs | 1:1 | 3:1 | 6:1 | 12:1 |
|---|---|---|---|---|
| HFB301001 | 1.2 | 0.7 | 0.5 | 0.3 |
| Bmk 1 | 2 | 0.8 | 0.5 | NM |
| Bmk 2 | NS | NS | NS | NM |

NS: not significant;
NM: not measured

### Example 21 Effects of HFB301001 on Cytokine Release

The effects of HFB301001 on cytokine release were evaluated in vitro using fresh hPBMCs from 6 different donors with the antibody in both soluble and plate-bound formats.

Different concentrations (3, 30 or 300 μg/ml) of HFB301001 or medium/control antibodies/mitogens were either coated at 4° C. overnight or added freshly to 96-well U bottom plates. 100 μl of a fresh PBMC suspension (2×10⁵ cells/well) were added to each well. The plates were incubated in a 37° C. 5% CO₂ incubator for 48 hrs. Supernatant was collected from each well and stored at −80° C. before analysis. The concentration of different cytokines in the culture supernatant was determined with BD™ CBA Human Th1/Th2 Cytokine Kit II (IL-2, IL-4, IL-6, IL-10, TNFα and IFNγ) according to the manufacturer's instructions.

When tested in the soluble format, minimal signals were detected in the negative controls-blank (complete medium only) or hIgG1 isotype control for all analytes in PBMCs from all donors, while positive controls PHA at 5 μg/ml or LPS at 10 μg/ml induced significantly increased release of IL-2 (PHA only), IL-4 (PHA only), IL-6, IL-10, TNFα (PHA only) and IFNγ (PHA only) in all or most donor's PBMCs. Similar to negative controls, minimal signals were detected in the presence of HFB301001 at 3, 30 or 300 μg/ml concentration in PBMCs from all 6 donors, suggesting low risk of HFB301001-induced cytokine release at the concentrations tested in the soluble format in vitro cytokine release assay.

When tested in the plate-bound format, minimal signals were detected in blank control. However, in the presence of hIgG1 isotype control, IL-4, IL-6 and TNFα, but not IL-2, IL-10 or IFNγ, were induced in all donors' PBMCs, which may be attributable to Fc receptor engagement by hIgG1. Two anti-hCD3 antibodies (OKT3 at 10 μg/ml and UCHT1 at 5 μg/ml) were used as positive controls and induced markedly higher cytokine release compared with blank control in all or most donors' PBMCs. Minimal signals were detected in the presence of HFB301001 at 3 μg/ml concentration in PBMCs from all or most of 6 donors, while IL-4, IL-6 and TNFα release were detected in the presence of HFB301001 at 30 or 300 μg/ml with dose dependency. Importantly, the level and pattern of cytokine release induced by HFB301001 at 300 μg/ml showed comparable or lower levels of cytokine release, as compared to hIgG1 isotype control at the same concentration, suggesting no increased risk of HFB301001-induced cytokine release over isotype control hIgG1 in the plate-bound format in vitro cytokine release assay.

Under the current experimental conditions, soluble HFB301001 at 3 to 300 μg/ml concentration did not induce significant cytokine release in PBMCs from all 6 donors, while plate bound HFB301001 at 300 μg/ml showed comparable or lower levels of cytokine release, as compared to hIgG1 isotype control at the same concentration.

### Example 22 Toxicology Studies of HFB301001

In all mouse studies conducted to date, HFB301001 was well tolerated without any sign of treatment-related toxicity. In hOX40 knockin mice, higher clearance and shorter T1/2 were observed for HFB301001 than WT mice, suggesting a possible target-mediated drug disposition.

In single dose toxicology study in cynomolgus monkeys, HFB301001 was well tolerated at all dose levels, and no apparent findings were observed at the highest dose of 100 mg/kg, suggesting 100 mg/kg to be below the maximum-tolerated dose (MTD). No gender-related differences were observed in systemic exposure to HFB301001. AUC₀₋ₜ and C_{end} values dose-proportionally increased in the range of 1 to 100 mg/kg. HFB301001 exhibited a typical mAb pharmacokinetic profile. Although a prolonged activated partial thromboplastin time and prothrombin time and an increase in total bilirubin concentration were observed in 1 female at 100 mg/kg compared to pre-dosing, and a moderate increase in total protein concentration also was noted in all animals, these changes may not be considered the test article related effects due to the lack of a concurrent control group and no dose-response relationships observed. Similarly, equivocal results of lower anti-KLH immunoglobulin titers-induced by HFB301001, which were below or in the lower end of historical control values, may not be considered test article-related effects due to lack of concurrent control group.

In a repeated dosing, GLP toxicology study was performed in male and female cynomolgus monkeys receiving weekly 1-hour intravenous infusions for 4 weeks followed by 4 weeks of recovery. Potential anti-drug antibody (ADA) was detected at the end of the dosing period in two males at 10 mg/kg, inducing a significant decrease in exposure to HFB301001 in one of them. No ADA was detected in other animals at the end of the dosing period. The weekly administration of HFB301001 was well-tolerated in all animals and did not induce any relevant sign of toxicity. Consequently, under the experimental conditions of this study, the no-observed-adverse-effect level (NOAEL) for HFB301001 was considered to be 100 mg/kg in both sexes, the highest dose tested, corresponding at the end of the dosing period to a male and female mean AUCₗₐₛₜ of 380500 μg h/mL.

Taken together, HFB301001 did not induce significant toxicity findings in all mouse studies or the NHP non-GLP single dose and the 1-month repeated-dose GLP toxicity study at doses up to the highest dose of 100 mg/kg. HFB301001 has a wide margin of safety (MOS) for the proposed FIH starting dose (5 mg flat monthly dosing).

### REFERENCE

Wang, R., Gao, C., Raymond, M., Dito, G, Kabbabe, D., Shao, X., Hilt, E., Sun, Y, Pak, L, Gutierrez, M., Melero, L, Spreafico, A., Carvajal, R., Ong, M., Olszanski, A., Milburn, C., Thudium, K., Yang, Z., Feng, Y, Fracasso, P., Korman, A., Aanur, P., Huang, S., Quigley, M. (2019). An Integrative Approach to Inform Optimal Administration of OX40 Agonist Antibodies in Patients with Advanced Solid Tumors, Clinical Cancer Research, dx.doi.org/10.1158/1078-0432.CCR-19-0526.

The inventive subject matter being thus described, it will be obvious that the same may be modified or varied in many ways. Such modifications and variations are not to be regarded as a departure from the spirit and scope of the inventive subject matter, and all such modifications and variations are intended to be included within the scope of the following claims.

### Sequence Lists:

| SEQ ID | Name | Sequence |
|---|---|---|
| SEQ ID NO: 1 | Amino acid | GFTFSSYA |
| | sequence of CDR1 | |
| | domain of | |
| | HFB10-1E1hG1 | |
| | heavy chain | |
| | variable region | |
| SEQ ID NO: 2 | Amino acid | ISGSGGST |
| | sequence of CDR2 | |
| | domain of | |
| | HFB10-1E1hG1 | |
| | heavy chain | |
| | variable region | |
| SEQ ID NO: 3 | Amino acid | ARDLSSSWYLDAFDI |
| | sequence of CDR3 | |
| | domain of | |
| | HFB10-1E1hG1 | |
| | heavy chain | |
| | variable region | |
| SEQ ID NO: 4 | Amino acid | EVQLLESGGGLVQPGGSLRLSCAASGFTFSSYAMSWVR |
| | sequence of | QAPGKGLEWVSAISGSGGSTYYADSVKGRFTISRDNSK |
| | HFB10-1E1hG1 | NTLYLQMNSLRAEDTAVYYCARDLSSSWYLDAFDIWGQ |
| | heavy chain | GTMVTVSS |
| | variable region | |
| SEQ ID NO: 5 | Amino acid | ASTKGPSVFPLAPSSKSTSGGTAALGCLVKDYFPEPVT |
| | sequence of | VSWNSGALTSGVHTFPAVLQSSGLYSLSSVVTVPSSSL |
| | HFB10-1E1hG1 | GTQTYICNVNHKPSNTKVDKKVEPKSCDKTHTCPPCPA |
| | heavy chain | PELLGGPSVFLFPPKPKDTLMISRTPEVTCVVVDVSHE |
| | constant region | DPEVKFNWYVDGVEVHNAKTKPREEQYNSTYRVVSVLT |
| | | VLHQDWLNGKEYKCKVSNKALPAPIEKTISKAKGQPRE |
| | | PQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 6 | Amino acid | MDLLHKNMKHLWFFLLLVAAPRWVLS |
| | sequence of | |
| | HFB10-1E1hG1 | |
| | heavy chain signal | |
| | peptide | |
| SEQ ID NO: 7 | Full-length amino | EVQLLESGGGLVQPGGSLRLSCAASGFTFSSYAMSWVR |
| | acid sequence of | QAPGKGLEWVSAISGSGGSTYYADSVKGRFTISRDNSK |
| | HFB10-1E1hG1 | NTLYLQMNSLRAEDTAVYYCARDLSSSWYLDAFDIWGQ |
| | heavy chain | GTMVTVSS |
| | (without signal | ASTKGPSVFPLAPSSKSTSGGTAALGCLVKDYFPEPVT |
| | peptide) | VSWNSGALTSGVHTFPAVLQSSGLYSLSSVVTVPSSSL |
| | | GTQTYICNVNHKPSNTKVDKKVEPKSCDKTHTCPPCPA |
| | | PELLGGPSVFLFPPKPKDTLMISRTPEVTCVVVDVSHE |
| | | DPEVKFNWYVDGVEVHNAKIKPREEQYNSTYRVVSVLT |
| | | VLHQDWLNGKEYKCKVSNKALPAPIEKTISKAKGQPRE |
| | | PQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 8 | Full-length amino | MDLLHKNMKHLWFFLLLVAAPRWVLS |
| | acid sequence of | EVQLLESGGGLVQPGGSLRLSCAASGFTFSSYAMSWVR |
| | HFB10-1E1hG1 | QAPGKGLEWVSAISGSGGSTYYADSVKGRFTISRDNSK |
| | heavy chain (with | NTLYLQMNSLRAEDTAVYYCARDLSSSWYLDAFDIWGQ |
| | signal peptide) | GTMVTVSS |
| | | ASTKGPSVFPLAPSSKSTSGGTAALGCLVKDYFPEPVT |
| | | VSWNSGALTSGVHTFPAVLQSSGLYSLSSVVTVPSSSL |
| | | GTQTYICNVNHKPSNTKVDKKVEPKSCDKTHTCPPCPA |
| | | PELLGGPSVFLFPPKPKDTLMISRTPEVTCVVVDVSHE |
| | | DPEVKFNWYVDGVEVHNAKTKPREEQYNSTYRVVSVLT |
| | | VLHQDWLNGKEYKCKVSNKALPAPIEKTISKAKGQPRE |
| | | PQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 9 | Amino acid | QGISSW |
| | sequence of CDR1 | |
| | domain of | |
| | HFB10-1E1hG1 | |
| | light chain | |
| | variable region | |
| SEQ ID NO: 10 | Amino acid | AAS |
| | sequence of CDR2 | |
| | domain of | |
| | HFB10-1E1hG1 | |
| | light chain | |
| | variable region | |
| SEQ ID NO: 11 | Amino acid | QQANSFPLT |
| | sequence of CDR3 | |
| | domain of | |
| | HFB10-1E1hG1 | |
| | light chain | |
| | variable region | |
| SEQ ID NO: 12 | Amino acid | DIQMTQSPSSVSASVGDRVTITCRASQGISSWLAWYQQ |
| | sequence of | KPGKAPKLLIYAASSLQSGVPSRFSGSGSGTDFTLTIS |
| | HFB10-1E1hG1 | SLQPEDFATYYCQQANSFPLTFGGGTKLEIK |
| | light chain | |
| | variable region | |
| SEQ ID NO: 13 | Amino acid | RTVAAPSVFIFPPSDEQLKSGTASVVCLLNNFYPREAK |
| | sequence of | VQWKVDNALQSGNSQESVTEQDSKDSTYSLSSTLTLSK |
| | HFB10-1E1hG1 | ADYEKHKVYACEVTHQGLSSPVTKSFNRGEC |
| | light chain | |
| | constant region | |
| SEQ ID NO: 14 | Amino acid | MDLLHKNMKHLWFFLLLVAAPRWVLS |
| | sequence of | |
| | HFB10-1E1hG1 | |
| | light chain signal | |
| | peptide | |
| SEQ ID NO: 15 | Full-length amino | DIQMTQSPSSVSASVGDRVTITCRASQGISSWLAWYQQ |
| | acid sequence of | KPGKAPKLLIYAASSLQSGVPSRFSGSGSGTDFTLTIS |
| | HFB10-1E1hG1 | SLQPEDFATYYCQQANSFPLTFGGGTKLEIK |
| | light chain | RTVAAPSVFIFPPSDEQLKSGTASVVCLLNNFYPREAK |
| | (without signal | VQWKVDNALQSGNSQESVTEQDSKDSTYSLSSTLTLSK |
| | peptide) | ADYEKHKVYACEVTHQGLSSPVTKSFNRGEC |
| SEQ ID NO: 16 | Full-length amino | MDLLHKNMKHLWFFLLLVAAPRWVLS |
| | acid sequence of | DIQMTQSPSSVSASVGDRVTITCRASQGISSWLAWYQQ |
| | HFB10-1E1hG1 | KPGKAPKLLIYAASSLQSGVPSRFSGSGSGTDFTLTIS |
| | light chain (with | SLQPEDFATYYCQQANSFPLTFGGGTKLEIK |
| | signal peptide) | RTVAAPSVFIFPPSDEQLKSGTASVVCLLNNFYPREAK |
| | | VQWKVDNALQSGNSQESVTEQDSKDSTYSLSSTLTLSK |
| | | ADYEKHKVYACEVTHQGLSSPVTKSFNRGEC |
| SEQ ID NO: 17 | Nucleotide coding | GGCTTCACCTTCTCCTCTTACGCC |
| | sequence of CDR1 | |
| | domain of | |
| | HFB10-1E1hG1 | |
| | heavy chain | |
| | variable region | |
| SEQ ID NO: 18 | Nucleotide coding | ATCTCCGGCTCTGGCGGATCTACC |
| | sequence of CDR2 | |
| | domain of | |
| | HFB10-1E1hG1 | |
| | heavy chain | |
| | variable region | |
| SEQ ID NO: 19 | Nucleotide coding | GCCAGAGATCTGTCCTCCTCCTGGTATCTGGACGCCTT |
| | sequence of CDR3 | TGATATC |
| | domain of | |
| | HFB10-1E1hG1 | |
| | heavy chain | |
| | variable region | |
| SEQ ID NO: 20 | variable region | GAAGTTCAGCTGCTGGAATCTGGCGGAGGACTGGTTCA |
| | Nucleotide coding | GCCTGGCGGCTCTTTGAGACTGTCTTGTGCCGCCTCTG |
| | sequence of | GCTTCACCTTCTCCTCTTACGCCATGTCCTGGGTCCGA |
| | HFB10-1E1hG1 | CAGGCTCCTGGAAAAGGACTGGAATGGGTGTCCGCTAT |
| | heavy chain | CTCCGGCTCTGGCGGATCTACCTACTACGCCGACTCCG |
| | | TGAAGGGCAGATTCACCATCAGCCGGGACAACTCCAAG |
| | | AACACCCTGTACCTGCAGATGAACTCCCTGAGAGCCGA |
| | | GGACACCGCCGTGTACTACTGTGCCAGAGATCTGTCCT |
| | | CCTCCTGGTATCTGGACGCCTTTGATATCTGGGGCCAG |
| | | GGCACCATGGTCACCGTGTCCTCT |
| | | GCTTCTACCAAGGGACCCTCTGTGTTCCCTCTGGCTCC |
| | | TTCCTCTAAATCCACCTCTGGTGGCACCGCTGCTCTGG |
| | | GCTGTCTGGTCAAGGATTACTTCCCTGAGCCTGTGACC |
| | | GTGTCTTGGAACTCTGGTGCTCTGACCTCCGGCGTGCA |
| | | CACATTCCCTGCTGTGCTGCAGTCCTCTGGCCTGTACT |
| | | CTCTGTCCTCTGTCGTGACCGTGCCTTCTAGCTCTCTG |
| | | GGCACCCAGACCTACATCTGCAACGTGAACCACAAGCC |
| | | TTCCAACACCAAGGTGGACAAGAAGGTGGAACCCAAGT |
| | | CCTGCGACAAGACCCACACCTGTCCTCCATGTCCAGCT |
| SEQ ID NO: 21 | Nucleotide coding | CCAGAACTGCTCGGCGGACCTTCCGTGTTCCTGTTTCC |
| | sequence of | TCCAAAGCCTAAGGACACCCTGATGATCTCTCGGACCC |
| | HFB10-1E1hG1 | CTGAAGTGACCTGCGTGGTGGTGGATGTGTCTCACGAG |
| | heavy chain | GACCCAGAAGTGAAGTTCAATTGGTACGTGGACGGCGT |
| | constant region | GGAAGTGCACAACGCCAAGACCAAGCCTAGAGAGGAAC |
| | | AGTACAACTCCACCTACAGAGTGGTGTCCGTGCTGACC |
| | | GTGCTGCACCAGGATTGGCTGAACGGCAAAGAGTACAA |
| | | GTGCAAGGTGTCCAACAAGGCCCTGCCTGCTCCTATCG |
| | | AAAAGACCATCTCTAAGGCCAAGGGCCAGCCTCGCGAA |
| | | CCTCAGGTTTACACCCTGCCTCCAAGCCGGGAAGAGAT |
| | | GACCAAGAACCAGGTGTCCCTGACCTGCCTGGTTAAGG |
| | | GCTTCTACCCCTCCGATATCGCCGTGGAATGGGAGTCT |
| | | AACGGCCAGCCTGAGAACAACTACAAGACAACCCCTCC |
| | | TGTGCTGGACTCCGACGGCTCATTCTTCCTGTACTCCA |
| | | AGCTGACAGTGGACAAGTCCAGATGGCAGCAGGGCAAC |
| | | GTGTTCTCCTGTTCTGTGATGCACGAGGCCCTGCACAA |
| | | CCACTACACACAGAAGTCCCTGTCTCTGTCCCCTGGCA |
| | | AG |
| SEQ ID NO: 22 | Nucleotide coding | ATGGACCTGCTGCACAAGAACATGAAGCACCTGTGGTT |
| | sequence of | CTTTCTGCTGCTGGTGGCCGCTCCTAGATGGGTGCTGT |
| | HFB10-1E1hG1 | CT |
| | heavy chain signal | |
| | peptide | |
| SEQ ID NO: 23 | Full-length | GAAGTTCAGCTGCTGGAATCTGGCGGAGGACTGGTTCA |
| | nucleotide coding | GCCTGGCGGCTCTTTGAGACTGTCTTGTGCCGCCTCTG |
| | sequence of | GCTTCACCTTCTCCTCTTACGCCATGTCCTGGGTCCGA |
| | HFB10-1E1hG1 | CAGGCTCCTGGAAAAGGACTGGAATGGGTGTCCGCTAT |
| | heavy chain | CTCCGGCTCTGGCGGATCTACCTACTACGCCGACTCCG |
| | (without signal | TGAAGGGCAGATTCACCATCAGCCGGGACAACTCCAAG |
| | peptide) | AACACCCTGTACCTGCAGATGAACTCCCTGAGAGCCGA |
| | | GGACACCGCCGTGTACTACTGTGCCAGAGATCTGTCCT |
| | | CCTCCTGGTATCTGGACGCCTTTGATATCTGGGGCCAG |
| | | GGCACCATGGTCACCGTGTCCTCTGCTTCTACCAAGGG |
| | | ACCCTCTGTGTTCCCTCTGGCTCCTTCCTCTAAATCCA |
| | | CCTCTGGTGGCACCGCTGCTCTGGGCTGTCTGGTCAAG |
| | | GATTACTTCCCTGAGCCTGTGACCGTGTCTTGGAACTC |
| | | TGGTGCTCTGACCTCCGGCGTGCACACATTCCCTGCTG |
| | | TGCTGCAGTCCTCTGGCCTGTACTCTCTGTCCTCTGTC |
| | | GTGACCGTGCCTTCTAGCTCTCTGGGCACCCAGACCTA |
| | | CATCTGCAACGTGAACCACAAGCCTTCCAACACCAAGG |
| | | TGGACAAGAAGGTGGAACCCAAGTCCTGCGACAAGACC |
| | | CACACCTGTCCTCCATGTCCAGCTCCAGAACTGCTCGG |
| | | CGGACCTTCCGTGTTCCTGTTTCCTCCAAAGCCTAAGG |
| | | ACACCCTGATGATCTCTCGGACCCCTGAAGTGACCTGC |
| | | GTGGTGGTGGATGTGTCTCACGAGGACCCAGAAGTGAA |
| | | GTTCAATTGGTACGTGGACGGCGTGGAAGTGCACAACG |
| | | CCAAGACCAAGCCTAGAGAGGAACAGTACAACTCCACC |
| | | TACAGAGTGGTGTCCGTGCTGACCGTGCTGCACCAGGA |
| | | TTGGCTGAACGGCAAAGAGTACAAGTGCAAGGTGTCCA |
| | | ACAAGGCCCTGCCTGCTCCTATCGAAAAGACCATCTCT |
| | | AAGGCCAAGGGCCAGCCTCGCGAACCTCAGGTTTACAC |
| | | CCTGCCTCCAAGCCGGGAAGAGATGACCAAGAACCAGG |
| | | TGTCCCTGACCTGCCTGGTTAAGGGCTTCTACCCCTCC |
| | | GATATCGCCGTGGAATGGGAGTCTAACGGCCAGCCTGA |
| | | GAACAACTACAAGACAACCCCTCCTGTGCTGGACTCCG |
| | | ACGGCTCATTCTTCCTGTACTCCAAGCTGACAGTGGAC |
| | | AAGTCCAGATGGCAGCAGGGCAACGTGTTCTCCTGTTC |
| | | TGTGATGCACGAGGCCCTGCACAACCACTACACACAGA |
| | | AGTCCCTGTCTCTGTCCCCTGGCAAG |
| SEQ ID NO: 24 | Full-length | ATGGACCTGCTGCACAAGAACATGAAGCACCTGTGGTT |
| | nucleotide coding | CTTTCTGCTGCTGGTGGCCGCTCCTAGATGGGTGCTGT |
| | sequence of | CT |
| | HFB10-1E1hG1 | GAAGTTCAGCTGCTGGAATCTGGCGGAGGACTGGTTCA |
| | heavy chain (with | GCCTGGCGGCTCTTTGAGACTGTCTTGTGCCGCCTCTG |
| | signal peptide) | GCTTCACCTTCTCCTCTTACGCCATGTCCTGGGTCCGA |
| | | CAGGCTCCTGGAAAAGGACTGGAATGGGTGTCCGCTAT |
| | | CTCCGGCTCTGGCGGATCTACCTACTACGCCGACTCCG |
| | | TGAAGGGCAGATTCACCATCAGCCGGGACAACTCCAAG |
| | | AACACCCTGTACCTGCAGATGAACTCCCTGAGAGCCGA |
| | | GGACACCGCCGTGTACTACTGTGCCAGAGATCTGTCCT |
| | | CCTCCTGGTATCTGGACGCCTTTGATATCTGGGGCCAG |
| | | GGCACCATGGTCACCGTGTCCTCTGCTTCTACCAAGGG |
| | | ACCCTCTGTGTTCCCTCTGGCTCCTTCCTCTAAATCCA |
| | | CCTCTGGTGGCACCGCTGCTCTGGGCTGTCTGGTCAAG |
| | | GATTACTTCCCTGAGCCTGTGACCGTGTCTTGGAACTC |
| | | TGGTGCTCTGACCTCCGGCGTGCACACATTCCCTGCTG |
| | | TGCTGCAGTCCTCTGGCCTGTACTCTCTGTCCTCTGTC |
| | | GTGACCGTGCCTTCTAGCTCTCTGGGCACCCAGACCTA |
| | | CATCTGCAACGTGAACCACAAGCCTTCCAACACCAAGG |
| | | TGGACAAGAAGGTGGAACCCAAGTCCTGCGACAAGACC |
| | | CACACCTGTCCTCCATGTCCAGCTCCAGAACTGCTCGG |
| | | CGGACCTTCCGTGTTCCTGTTTCCTCCAAAGCCTAAGG |
| | | ACACCCTGATGATCTCTCGGACCCCTGAAGTGACCTGC |
| | | GTGGTGGTGGATGTGTCTCACGAGGACCCAGAAGTGAA |
| | | GTTCAATTGGTACGTGGACGGCGTGGAAGTGCACAACG |
| | | CCAAGACCAAGCCTAGAGAGGAACAGTACAACTCCACC |
| | | TACAGAGTGGTGTCCGTGCTGACCGTGCTGCACCAGGA |
| | | TTGGCTGAACGGCAAAGAGTACAAGTGCAAGGTGTCCA |
| | | ACAAGGCCCTGCCTGCTCCTATCGAAAAGACCATCTCT |
| | | AAGGCCAAGGGCCAGCCTCGCGAACCTCAGGTTTACAC |
| | | CCTGCCTCCAAGCCGGGAAGAGATGACCAAGAACCAGG |
| | | TGTCCCTGACCTGCCTGGTTAAGGGCTTCTACCCCTCC |
| | | GATATCGCCGTGGAATGGGAGTCTAACGGCCAGCCTGA |
| | | GAACAACTACAAGACAACCCCTCCTGTGCTGGACTCCG |
| | | ACGGCTCATTCTTCCTGTACTCCAAGCTGACAGTGGAC |
| | | AAGTCCAGATGGCAGCAGGGCAACGTGTTCTCCTGTTC |
| | | TGTGATGCACGAGGCCCTGCACAACCACTACACACAGA |
| | | AGTCCCTGTCTCTGTCCCCTGGCAAG |
| SEQ ID NO: 25 | Nucleotide coding | CAGGGCATCTCTAGCTGG |
| | sequence of CDR1 | |
| | domain of | |
| | HFB10-1E1hG1 | |
| | light chain | |
| | variable region | |
| SEQ ID NO: 26 | Nucleotide coding | GCCAGTTCT |
| | sequence of CDR2 | |
| | domain of | |
| | HFB10-1E1hG1 | |
| | light chain | |
| | variable region | |
| SEQ ID NO: 27 | Nucleotide coding | CAGCAGGCCAACAGCTTCCCTCTGACC |
| | sequence of CDR3 | |
| | domain of | |
| | HFB10-1E1hG1 | |
| | light chain | |
| | variable region | |
| SEQ ID NO: 28 | Nucleotide coding | GACATCCAGATGACCCAGTCTCCATCCTCCGTGTCTGC |
| | sequence of | CTCTGTGGGCGACAGAGTGACCATCACCTGTAGAGCCT |
| | HFB10-1E1hG1 | CTCAGGGCATCTCTAGCTGGCTGGCCTGGTATCAGCAG |
| | light chain | AAGCCTGGAAAGGCCCCTAAGCTGCTGATCTACGCTGC |
| | variable region | CAGTTCTCTGCAGTCTGGCGTGCCCTCTAGATTCTCTG |
| | | GCTCTGGATCTGGCACCGACTTCACCCTGACAATCTCT |
| | | AGCCTGCAGCCTGAGGACTTCGCCACCTACTACTGTCA |
| | | GCAGGCCAACAGCTTCCCTCTGACCTTTGGCGGCGGAA |
| | | CAAAGCTGGAAATCAAG |
| SEQ ID NO: 29 | Nucleotide coding | AGAACCGTGGCTGCCCCTTCCGTGTTCATCTTCCCACC |
| | sequence of | ATCTGACGAGCAGCTGAAGTCCGGCACAGCTTCTGTCG |
| | HFB10-1E1hG1 | TGTGCCTGCTGAACAACTTCTACCCTCGGGAAGCCAAG |
| | light chain | GTGCAGTGGAAGGTGGACAATGCCCTGCAGTCCGGCAA |
| | constant region | CTCCCAAGAGTCTGTGACCGAGCAGGACTCCAAGGACT |
| | | CTACCTACAGCCTGTCCTCCACACTGACCCTGTCTAAG |
| | | GCCGACTACGAGAAGCACAAGGTGTACGCCTGTGAAGT |
| | | GACCCACCAGGGACTGTCTAGCCCCGTGACCAAGTCTT |
| | | TCAACAGAGGCGAGTGC |
| SEQ ID NO: 30 | Nucleotide coding | ATGGACCTGCTGCACAAGAACATGAAGCACCTGTGGTT |
| | sequence of | CTTTCTGCTGCTGGTGGCCGCTCCTAGATGGGTGCTGT |
| | HFB10-1E1hG1 | CT |
| | light chain signal | |
| | peptide | |
| SEQ ID NO: 31 | Full-length | GACATCCAGATGACCCAGTCTCCATCCTCCGTGTCTGC |
| | nucleotide coding | CTCTGTGGGCGACAGAGTGACCATCACCTGTAGAGCCT |
| | sequence of | CTCAGGGCATCTCTAGCTGGCTGGCCTGGTATCAGCAG |
| | HFB10-1E1hG1 | AAGCCTGGAAAGGCCCCTAAGCTGCTGATCTACGCTGC |
| | light chain | CAGTTCTCTGCAGTCTGGCGTGCCCTCTAGATTCTCTG |
| | (without signal | GCTCTGGATCTGGCACCGACTTCACCCTGACAATCTCT |
| | peptide) | AGCCTGCAGCCTGAGGACTTCGCCACCTACTACTGTCA |
| | | GCAGGCCAACAGCTTCCCTCTGACCTTTGGCGGCGGAA |
| | | CAAAGCTGGAAATCAAGAGAACCGTGGCTGCCCCTTCC |
| | | GTGTTCATCTTCCCACCATCTGACGAGCAGCTGAAGTC |
| | | CGGCACAGCTTCTGTCGTGTGCCTGCTGAACAACTTCT |
| | | ACCCTCGGGAAGCCAAGGTGCAGTGGAAGGTGGACAAT |
| | | GCCCTGCAGTCCGGCAACTCCCAAGAGTCTGTGACCGA |
| | | GCAGGACTCCAAGGACTCTACCTACAGCCTGTCCTCCA |
| | | CACTGACCCTGTCTAAGGCCGACTACGAGAAGCACAAG |
| | | GTGTACGCCTGTGAAGTGACCCACCAGGGACTGTCTAG |
| | | CCCCGTGACCAAGTCTTTCAACAGAGGCGAGTGC |
| SEQ ID NO: 32 | Full-length | ATGGACCTGCTGCACAAGAACATGAAGCACCTGTGGTT |
| | nucleotide coding | CTTTCTGCTGCTGGTGGCCGCTCCTAGATGGGTGCTGT |
| | sequence of | CT |
| | HFB10-1E1hG1 | GACATCCAGATGACCCAGTCTCCATCCTCCGTGTCTGC |
| | Light chain (with | CTCTGTGGGCGACAGAGTGACCATCACCTGTAGAGCCT |
| | signal peptide) | CTCAGGGCATCTCTAGCTGGCTGGCCTGGTATCAGCAG |
| | | AAGCCTGGAAAGGCCCCTAAGCTGCTGATCTACGCTGC |
| | | CAGTTCTCTGCAGTCTGGCGTGCCCTCTAGATTCTCTG |
| | | GCTCTGGATCTGGCACCGACTTCACCCTGACAATCTCT |
| | | AGCCTGCAGCCTGAGGACTTCGCCACCTACTACTGTCA |
| | | GCAGGCCAACAGCTTCCCTCTGACCTTTGGCGGCGGAA |
| | | CAAAGCTGGAAATCAAGAGAACCGTGGCTGCCCCTTCC |
| | | GTGTTCATCTTCCCACCATCTGACGAGCAGCTGAAGTC |
| | | CGGCACAGCTTCTGTCGTGTGCCTGCTGAACAACTTCT |
| | | ACCCTCGGGAAGCCAAGGTGCAGTGGAAGGTGGACAAT |
| | | GCCCTGCAGTCCGGCAACTCCCAAGAGTCTGTGACCGA |
| | | GCAGGACTCCAAGGACTCTACCTACAGCCTGTCCTCCA |
| | | CACTGACCCTGTCTAAGGCCGACTACGAGAAGCACAAG |
| | | GTGTACGCCTGTGAAGTGACCCACCAGGGACTGTCTAG |
| | | CCCCGTGACCAAGTCTTTCAACAGAGGCGAGTGC |
| SEQ ID NO: 33 | | MCVGARRLGRGPCAALLLLGLGLSTVTGLHCVGDTYPS |
| | | NDRCCHECRPGNGMVSRCSRSQNTVCRPCGPGFYNDVV |
| | | SSKPCKPCTWCNLRSGSERKQLCTATQDTVCRCRAGTQ |
| | | PLDSYKPGVDCAPCPPGHFSPGDNQACKPWTNCTLAGK |
| | | HTLQPASNSSDAICEDRDPPATQPQETQGPPARPITVQ |
| | | PTEAWPRTSQGPSTRPVEVPGGRAVAAILGLGLVLGLL |
| | | GPLAILLALYLLRRDQRLPPDAHKPPGGGSFRTPIQEE |
| | | QADAHSTLAKI |
| SEQ ID NO: 34 | | CSRSQNTVCRPCGPGFYND |

1. An isolated monoclonal antibody, or an antigen-binding fragment thereof, wherein said monoclonal antibody or antigen-binding fragment thereof specifically binds to OX40 (e.g., human OX40) at an epitope comprising, consisting essentially of, or consisting of SEQ ID NO: 34, as an OX40 agonist, and does not substantially affect OX40-OX40L interaction/binding.

2. The isolated monoclonal antibody, or antigen-binding fragment thereof of claim 1, which induces NF-kB-mediated OX40 signaling in the presence or absence of OX40L.

3. The isolated monoclonal antibody, or antigen-binding fragment thereof of claim 1 or 2, which does not inhibit, or enhances, (endogenous) OX40L-induced OX40 signaling (e.g., NF-kB signaling).

4. The isolated monoclonal antibody, or antigen-binding fragment thereof of claim 3, wherein OX40L is at least about 50 nM, 60 nM, 70 nM, 80 nM, 90 nM or more.

5. The isolated monoclonal antibody, or antigen-binding fragment thereof of any one of claims 1-4, which binds to activated primary (human or cynomolgus monkey) CD4⁺CD3⁺ T cells with an EC50 value of about 0.01-5 nM, 0.1-0.3 nM, or 0.5-1.5 nM (e.g., 0.65 or 1.36 nM).

6. The isolated monoclonal antibody, or antigen-binding fragment thereof of any one of claims 1-5, which increases total surface level OX40 expression on active (e.g., CD3/CD28-stimulated) peripheral human CD4⁺ T cells in a dose-dependent manner (e.g., with a maximum OX40 expression stimulated by about 1 nM of said antibody or fragment thereof).

7. The isolated monoclonal antibody, or antigen-binding fragment thereof of any one of claims 1-6, which dose-dependently augment T-cell activation.

8. The isolated monoclonal antibody, or antigen-binding fragment thereof of claim 6, which increases cytokine (e.g., IL-2) production in activated human PBMCs;
optionally, said human PBMCs are stimulated by Staphylococcus aureus enterotoxin A (SEA) at 1 or 10 ng/mL.

9. The isolated monoclonal antibody, or antigen-binding fragment thereof of any one of claims 1-8, which dose-dependently induce CD16-mediated ADCC in effector cells in the presence of OX-40-expressing target cells, e.g., with an EC50 of about 0.3-1.2 nM at effector to target cell (E:T) ratios of 12:1 to 1:1.

10. The isolated monoclonal antibody or antigen-binding fragment thereof of any one of claims 1-9, which does not induce significant cytokine release in primary PBMCs at up to 300 μg/mL

11. The isolated monoclonal antibody, or antigen-binding fragment thereof of any one of claims 1-10, which does not induce significant toxicity in mouse at a maximum tolerable dose of at least about 100 mg/kg.

12. The isolated monoclonal antibody, or antigen-binding fragment thereof of any one of claims 1-11, comprising:
a) a heavy chain variable region (VH) comprising a heavy chain CDR1 of SEQ ID NO: 1, a heavy chain CDR2 of SEQ ID NO: 2, and a heavy chain CDR3 of SEQ ID NO: 3, and,
b) a light chain variable region (VL) comprising a light chain CDR1 of SEQ ID NO: 9, a light chain CDR2 of SEQ ID NO: 10, and a light chain CDR3 of SEQ ID NO: 11.

13. The isolated monoclonal antibody, or antigen-binding fragment thereof of claim 12, wherein:
a) the VH comprise the amino acid sequence of SEQ ID NO: 4, or an amino acid sequence sharing at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 4; and,
b) the VL comprises the amino acid sequence of SEQ ID NO: 12, or an amino acid sequence sharing at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 12.

14. The isolated monoclonal antibody, or antigen-binding fragment thereof of claim 12 or 13, further comprising a heavy chain constant region (CH) and a light chain constant region (CL).

15. The isolated monoclonal antibody, or antigen-binding fragment thereof of claim 14, wherein:
1) said CH comprises the amino acid sequence of SEQ ID NO: 5, or an amino acid sequence sharing at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 5; and/or,
2) said CL comprises the amino acid sequence of SEQ ID NO: 13, or an amino acid sequence sharing at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 13.

16. The isolated monoclonal antibody, or antigen-binding fragment thereof of any one of claims 12-15, further comprising a heavy chain signal sequence N-terminal to said VH, and/or a light chain signal sequence N-terminal to said VL.

17. The isolated monoclonal antibody, or antigen-binding fragment thereof of claim 16, wherein:
1) said heavy chain signal sequence comprises the amino acid sequence of SEQ ID NO: 6, or an amino acid sequence sharing at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 6; and/or,
2) said light chain signal sequence comprises the amino acid sequence of SEQ ID NO: 14, or an amino acid sequence sharing at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 14.

18. The isolated monoclonal antibody, or antigen-binding fragment thereof of any one of claims 12-17, wherein said antibody is an IgG, such as IgG1.

19. The isolated monoclonal antibody, or antigen-binding fragment thereof of any one of claims 12-18, wherein said antigen-binding fragment is Fab, Fab′, F(ab′)₂, Fv, scFv, or sdAb.

20. An antibody-drug conjugate (ADC), comprising the monoclonal antibody or antigen-binding fragment thereof of any one of claims 1-17, and a therapeutic moiety, optionally, said monoclonal antibody or antigen-binding fragment thereof is fused to said therapeutic moiety which is a protein.

21. A polynucleotide encoding the monoclonal antibody or antigen-binding fragment thereof of any one of claims 1-19.

22. The polynucleotide of claim 21, comprising a coding sequence for the heavy chain variable region having the polynucleotide sequence of SEQ ID NO: 20, and/or a coding sequence for the light chain variable region having the polynucleotide sequence of SEQ ID NO: 28.

23. The polynucleotide of claim 21 or 22, further comprising a coding sequence for the heavy chain constant region having the polynucleotide sequence of SEQ ID NO: 21, and/or a coding sequence for the light chain constant region having the polynucleotide sequence of SEQ ID NO: 29.

24. A vector (such as an expression vector for mammalian expression) comprising the polynucleotide of any one of claims 21-23.

25. A host cell comprising the polynucleotide of any one of claims 21-23, or the vector of claim 24.

26. A method of producing the monoclonal antibody or antigen-binding fragment thereof of any one of claims 1-19, the method comprising culturing the host cell of claim 25 in a medium under a condition suitable for antibody expression in said host cell, optionally further comprising harvesting, collecting, isolating or purifying said monoclonal antibody or antigen-binding fragment thereof from the medium.

27. A pharmaceutical composition comprising the monoclonal antibody or antigen-binding fragment thereof any one of claims 1-19, the ADC of claim 20, the polynucleotide of any one of claims 21-23, or the vector of claim 24; and a pharmaceutically acceptable carrier or excipient.

28. The pharmaceutical composition of claim 27, further comprising a second therapeutic agent, or for administration with a second therapeutic agent.

29. A method of treating cancer in a patient in need thereof, the method comprises administering to the patient a therapeutically effective amount of an OX40 agonist antibody or an antigen-binding fragment thereof, wherein said antibody or antigen-binding fragment thereof specifically binds to OX40 (e.g., human OX40) at an epitope comprising, consisting essentially of, or consisting of SEQ ID NO: 34, as an OX40 agonist, and does not substantially affect OX40-OX40L interaction/binding.

30. A method of treating cancer in a patient in need thereof, the method comprises administering to the patient the monoclonal antibody or antigen-binding fragment thereof any one of claims 1-19, the ADC of claim 20, the polynucleotide of any one of claims 21-23, the vector of claim 24, or the pharmaceutical composition of claim 27 or 28.

31. Use of the monoclonal antibody or antigen-binding fragment thereof any one of claims 1-19, the ADC of claim 20, the polynucleotide of any one of claims 21-23, the vector of claim 24, or the pharmaceutical composition of claim 27 or 28, in the manufacture of a medicament for treating cancer.

32. The method of claim 29 or 30, or the use of claim 31, wherein the cancer is selected from the group consisting of: squamous cell carcinoma (e.g., epithelial squamous cell carcinoma), lung cancer (including small cell lung cancer, non-small cell lung cancer, adenocarcinoma of the lung, and squamous cell carcinoma of the lung), peritoneal cancer, hepatocellular carcinoma, gastric cancer (including gastrointestinal cancer and gastrointestinal stromal cancer), pancreatic cancer, glioblastoma, cervical cancer, ovarian cancer, liver cancer, bladder cancer, urethral cancer, liver tumor, breast cancer, colon cancer, rectal cancer, colorectal cancer, endometrial or uterine cancer, salivary gland cancer, kidney cancer, prostate cancer, vulvar cancer, thyroid cancer, liver cancer, anal cancer, penile cancer, melanoma, superficial spreading melanoma, malignant lentigines melanoma, acral melanoma, nodular melanoma, multiple myeloma and B-cell lymphoma, chronic lymphocytic leukemia (CLL), acute lymphoblastic leukemia (ALL), hairy cell leukemia, chronic myeloblastic leukemia, post-transplant lymphoproliferative disorder (PTLD), abnormal vascular proliferation related to scar nevus, edema (for example, related to brain tumors) and Meigs syndrome, brain tumors and brain cancers, head and neck cancers, and related metastases.

33. Use of any one of the anti-OX40 monoclonal antibody or antigen-binding fragment thereof of claims 1-19, or the antibody-drug conjugate according to claim 20, or the pharmaceutical composition according to claim 27 or 28, in one or more of: inhibiting Treg function (for example, inhibiting the suppressive function of Treg), killing OX40-expressing cells (for example, cells expressing high levels of OX40), increasing effector T cell function and/or increasing memory T cell function, reducing tumor immunity, enhancing T cell function, and/or depleting cells expressing OX40.

34. Use of any one of the anti-OX40 monoclonal antibody or antigen-binding fragment thereof of claims 1-19, or the antibody-drug conjugate according to claim 20, or the pharmaceutical composition according to claim 27 or 28, in the manufacture of a medicament for one or more of: inhibiting Treg function (for example, inhibiting the suppressive function of Treg), killing OX40-expressing cells (for example, cells expressing high levels of OX40), increasing effector T cell function and/or increasing memory T cell function, reducing tumor immunity, enhancing T cell function, and/or depleting cells expressing OX40.

35. A kit comprising the anti-OX40 antibody or antigen-binding fragment thereof according to any one of claims 1-19, the antibody-drug conjugate according to claim 20, or the pharmaceutical composition according to claim 27 or 28, preferably further including an administration device.

36. A method to activate a T cell (such as a CD4⁺ T cell), to inhibit a Treg cell, to kill an OX40-expressing target cell, to enhance effector or memory T cell function, or to reduce tumor-induced immuno-suppression, comprising contacting the T cell, the Treg cell, the OX40-expressing target cell, the effector or memory T cell, respectively, with an OX40 agonist antibody, such as the monoclonal antibody or antigen-binding fragment thereof any one of claims 1-19, wherein the agonist antibody binds to a human OX40 epitope comprising, consisting essentially of, or consisting of the amino acid sequence of SEQ ID NO: 34.


SEQUENCE LISTING (34 sequences)

SEQ ID NO: 1 - protein, 8 aa, Artificial Sequence
  HFB10-1E1hG1 VH CDR1 A.A. Sequence
GFTFSSYA

SEQ ID NO: 2 - protein, 8 aa, Artifical Sequence
  HFB10-1E1hG1 VH CDR2 A.A. Sequence
ISGSGGST

SEQ ID NO: 3 - protein, 15 aa, Artificial Sequence
  HFB10-1E1hG1 VH CDR3 A.A. Sequence
ARDLSSSWYLDAFDI

SEQ ID NO: 4 - protein, 122 aa, Artificial Sequence
  HFB10-1E1hG1 VH A.A. Sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLSSSWYLDAFDIWGQGTMVTVSS

SEQ ID NO: 5 - protein, 330 aa, Artificial Sequence
  HFB10-1E1hG1 HC Constant Region A.A. Sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 26 aa, Artificial Sequence
  HFB10-1E1hG1 HC Signal Peptide Sequence
MDLLHKNMKHLWFFLLLVAAPRWVLS

SEQ ID NO: 7 - protein, 452 aa, Artificial Sequence
  HFB10-1E1hG1 HC Full-Length A.A. Sequence (no Signal Peptide)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLSSSWYLDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 478 aa, Artificial Sequence
  HFB10-1E1hG1 HC Full-length A.A. Sequence (with Signal Peptide)
MDLLHKNMKHLWFFLLLVAAPRWVLSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLSSSWYLDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 6 aa, Artificial Sequence
  HFB10-1E1hG1 VL CDR1 A.A. Sequence
QGISSW

SEQ ID NO: 10 - protein, 3 aa, Artificial Sequence
  HFB10-1E1hG1 VL CDR2 A.A. Sequence
AAS

SEQ ID NO: 11 - protein, 9 aa, Artificial Sequence
  HFB10-1E1hG1 VL CDR3 A.A. Sequence
QQANSFPLT

SEQ ID NO: 12 - protein, 107 aa, Artificial Sequence
  HFB10-1E1hG1 VL A.A. Sequence
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKLEIK

SEQ ID NO: 13 - protein, 107 aa, Artificial Sequence [sequence omitted]

SEQ ID NO: 14 - protein, 26 aa, Artificial Sequence
  HFB10-1E1hG1 LC Signal Peptide Sequence
MDLLHKNMKHLWFFLLLVAAPRWVLS

SEQ ID NO: 15 - protein, 214 aa, Artificial Sequence
  HFB10-1E1hG1 LC Full-length A.A. Sequence (no Signal Peptide)
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 240 aa, Artificial Sequence
  HFB10-1E1hG1 LC Full-length Sequence (with Signal Peptide)
MDLLHKNMKHLWFFLLLVAAPRWVLSDIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 24 bp, Artificial Sequence
  HFB10-1E1hG1 VH CDR1 N.A. Sequence
ggcttcaccttctcctcttacgcc

SEQ ID NO: 18 - DNA, 24 bp, Artificial Sequence
  HFB10-1E1hG1 VH CDR2 N.A. Sequence
atctccggctctggcggatctacc

SEQ ID NO: 19 - DNA, 45 bp, Artificial Sequence
  HFB10-1E1hG1 VH CDR3 N.A. Sequence
gccagagatctgtcctcctcctggtatctggacgcctttgatatc

SEQ ID NO: 20 - DNA, 366 bp, Artificial Sequence
  HFB10-1E1hG1 VH N.A. Sequence
gaagttcagctgctggaatctggcggaggactggttcagcctggcggctctttgagactgtcttgtgccgcctctggcttcaccttctcctcttacgccatgtcctgggtccgacaggctcctggaaaaggactggaatgggtgtccgctatctccggctctggcggatctacctactacgccgactccgtgaagggcagattcaccatcagccgggacaactccaagaacaccctgtacctgcagatgaactccctgagagccgaggacaccgccgtgtactactgtgccagagatctgtcctcctcctggtatctggacgcctttgatatctggggccagggcaccatggtcaccgtgtcctct

SEQ ID NO: 21 - DNA, 990 bp, Artificial Sequence
  HFB10-1E1hG1 HC Constant Region N.A. Sequence
gcttctaccaagggaccctctgtgttccctctggctccttcctctaaatccacctctggtggcaccgctgctctgggctgtctggtcaaggattacttccctgagcctgtgaccgtgtcttggaactctggtgctctgacctccggcgtgcacacattccctgctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtccagctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctctaaggccaagggccagcctcgcgaacctcaggtttacaccctgcctccaagccgggaagagatgaccaagaaccaggtgtccctgacctgcctggttaagggcttctacccctccgatatcgccgtggaatgggagtctaacggccagcctgagaacaactacaagacaacccctcctgtgctggactccgacggctcattcttcctgtactccaagctgacagtggacaagtccagatggcagcagggcaacgtgttctcctgttctgtgatgcacgaggccctgcacaaccactacacacagaagtccctgtctctgtcccctggcaag

SEQ ID NO: 22 - DNA, 78 bp, Artificial Sequence
  HFB10-1E1hG1 HC Signal Peptide N.A. Sequence
atggacctgctgcacaagaacatgaagcacctgtggttctttctgctgctggtggccgctcctagatgggtgctgtct

SEQ ID NO: 23 - DNA, 1356 bp, Artificial Sequence
  HFB10-1E1hG1 HC Full-length N.A. Sequence (no Signal Peptide)
gaagttcagctgctggaatctggcggaggactggttcagcctggcggctctttgagactgtcttgtgccgcctctggcttcaccttctcctcttacgccatgtcctgggtccgacaggctcctggaaaaggactggaatgggtgtccgctatctccggctctggcggatctacctactacgccgactccgtgaagggcagattcaccatcagccgggacaactccaagaacaccctgtacctgcagatgaactccctgagagccgaggacaccgccgtgtactactgtgccagagatctgtcctcctcctggtatctggacgcctttgatatctggggccagggcaccatggtcaccgtgtcctctgcttctaccaagggaccctctgtgttccctctggctccttcctctaaatccacctctggtggcaccgctgctctgggctgtctggtcaaggattacttccctgagcctgtgaccgtgtcttggaactctggtgctctgacctccggcgtgcacacattccctgctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtccagctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctctaaggccaagggccagcctcgcgaacctcaggtttacaccctgcctccaagccgggaagagatgaccaagaaccaggtgtccctgacctgcctggttaagggcttctacccctccgatatcgccgtggaatgggagtctaacggccagcctgagaacaactacaagacaacccctcctgtgctggactccgacggctcattcttcctgtactccaagctgacagtggacaagtccagatggcagcagggcaacgtgttctcctgttctgtgatgcacgaggccctgcacaaccactacacacagaagtccctgtctctgtcccctggcaag

SEQ ID NO: 24 - DNA, 1434 bp, Artificial Sequence
  HFB10-1E1hG1 HC Full-length N.A. Sequence (with Signal Peptide)
atggacctgctgcacaagaacatgaagcacctgtggttctttctgctgctggtggccgctcctagatgggtgctgtctgaagttcagctgctggaatctggcggaggactggttcagcctggcggctctttgagactgtcttgtgccgcctctggcttcaccttctcctcttacgccatgtcctgggtccgacaggctcctggaaaaggactggaatgggtgtccgctatctccggctctggcggatctacctactacgccgactccgtgaagggcagattcaccatcagccgggacaactccaagaacaccctgtacctgcagatgaactccctgagagccgaggacaccgccgtgtactactgtgccagagatctgtcctcctcctggtatctggacgcctttgatatctggggccagggcaccatggtcaccgtgtcctctgcttctaccaagggaccctctgtgttccctctggctccttcctctaaatccacctctggtggcaccgctgctctgggctgtctggtcaaggattacttccctgagcctgtgaccgtgtcttggaactctggtgctctgacctccggcgtgcacacattccctgctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtccagctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctctaaggccaagggccagcctcgcgaacctcaggtttacaccctgcctccaagccgggaagagatgaccaagaaccaggtgtccctgacctgcctggttaagggcttctacccctccgatatcgccgtggaatgggagtctaacggccagcctgagaacaactacaagacaacccctcctgtgctggactccgacggctcattcttcctgtactccaagctgacagtggacaagtccagatggcagcagggcaacgtgttctcctgttctgtgatgcacgaggccctgcacaaccactacacacagaagtccctgtctctgtcccctggcaag

SEQ ID NO: 25 - DNA, 18 bp, Artificial Sequence
  HFB10-1E1hG1 VL CDR1 N.A. Sequence
cagggcatctctagctgg

SEQ ID NO: 26 - DNA, 9 bp, Artificial Sequence
  HFB10-1E1hG1 VL CDR2 N.A. Sequence
gccagttct

SEQ ID NO: 27 - DNA, 27 bp, Artificial Sequence
  HFB10-1E1hG1 VL CDR3 N.A. Sequence
cagcaggccaacagcttccctctgacc

SEQ ID NO: 28 - DNA, 321 bp, Artificial Sequence
  HFB10-1E1hG1 VL N.A. Sequence
gacatccagatgacccagtctccatcctccgtgtctgcctctgtgggcgacagagtgaccatcacctgtagagcctctcagggcatctctagctggctggcctggtatcagcagaagcctggaaaggcccctaagctgctgatctacgctgccagttctctgcagtctggcgtgccctctagattctctggctctggatctggcaccgacttcaccctgacaatctctagcctgcagcctgaggacttcgccacctactactgtcagcaggccaacagcttccctctgacctttggcggcggaacaaagctggaaatcaag

SEQ ID NO: 29 - DNA, 321 bp, Artificial Sequence
  HFB10-1E1hG1 LC Constant Region N.A. Sequence
agaaccgtggctgccccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaacagaggcgagtgc

SEQ ID NO: 30 - DNA, 78 bp, Artificial Sequence; identical to SEQ ID NO: 22
  HFB10-1E1hG1 LC Signal Peptide N.A. Sequence

SEQ ID NO: 31 - DNA, 642 bp, Artificial Sequence
  HFB10-1E1hG1 LC Full-length N.A. Sequence (no Signal Peptide)
gacatccagatgacccagtctccatcctccgtgtctgcctctgtgggcgacagagtgaccatcacctgtagagcctctcagggcatctctagctggctggcctggtatcagcagaagcctggaaaggcccctaagctgctgatctacgctgccagttctctgcagtctggcgtgccctctagattctctggctctggatctggcaccgacttcaccctgacaatctctagcctgcagcctgaggacttcgccacctactactgtcagcaggccaacagcttccctctgacctttggcggcggaacaaagctggaaatcaagagaaccgtggctgccccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaacagaggcgagtgc

SEQ ID NO: 32 - DNA, 720 bp, Artificial Sequence
  HFB10-1E1hG1 LC Full-length N.A. Sequence (with Signal Peptide)
atggacctgctgcacaagaacatgaagcacctgtggttctttctgctgctggtggccgctcctagatgggtgctgtctgacatccagatgacccagtctccatcctccgtgtctgcctctgtgggcgacagagtgaccatcacctgtagagcctctcagggcatctctagctggctggcctggtatcagcagaagcctggaaaggcccctaagctgctgatctacgctgccagttctctgcagtctggcgtgccctctagattctctggctctggatctggcaccgacttcaccctgacaatctctagcctgcagcctgaggacttcgccacctactactgtcagcaggccaacagcttccctctgacctttggcggcggaacaaagctggaaatcaagagaaccgtggctgccccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaacagaggcgagtgc

SEQ ID NO: 33 - protein, 277 aa, H. sapien
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 34 - protein, 19 aa, H. sapien
CSRSQNTVCRPCGPGFYND